(19) United States
(12) Patent Application Publication
(10) Pub. No.: US 2026/0007480 A1
(43) Pub. Date: Jan. 8, 2026

(54) MEDICAL MANIPULATOR SYSTEM, PROCESSOR, AND CONTROL METHOD

(71) Applicant: OLYMPUS CORPORATION, Tokyo (JP)

(72) Inventors: Masahiro FUJII, Tokyo (JP); Shiori YASUDA, Tokyo (JP); Ryohei OGAWA, Tokyo (JP)

(73) Assignee: OLYMPUS CORPORATION, Tokyo (JP)

(21) Appl. No.: 19/098,444

(22) Filed: Apr. 2, 2025

Related U.S. Application Data
(60) Provisional application No. 63/667,872, filed on Jul. 5, 2024.

(51) Int. Cl. A61B 34/30; A61B 1/00; A61B 1/01; A61B 1/018; A61B 1/04; A61B 34/10; A61B 34/20; A61M 25/01; A61M 25/06

(52) U.S. Cl. CPC A61B 34/30; A61B 1/00006; A61B 1/00009; A61B 1/01; A61B 1/018; A61B 1/04; A61B 2034/105; A61B 2034/2051; A61B 2034/2055; A61B 2034/301; A61M 25/0113; A61M 2025/0681

(57) ABSTRACT

A medical manipulator system includes an outer manipulator including an imager that captures an image, an inner manipulator that protrudes from a distal end surface of the outer manipulator, a driving device that controls the outer manipulator and the inner manipulator, and a processor. The processor calculates information regarding a work region that is an operable range of the inner manipulator with respect to each of a plurality of positions of the outer manipulator to acquire information regarding a plurality of the work regions. The processor performs drive control of the outer manipulator to move to a first position corresponding to a first work region selected from among the plurality of work regions based on the region of the treatment target, the three-dimensional shape information regarding the lumen, and the three-dimensional shape information regarding the outer manipulator.

### CROSS REFERENCE TO RELATED APPLICATIONS

This application is based upon and claims the benefit of priority to U.S. Provisional Patent Application No. 63/667,872 filed on Jul. 5, 2024, the entire contents of which are incorporated herein by reference.

### BACKGROUND OF THE INVENTION

A surgery system that controls a medical device such as an endoscope using a robot arm or the like has been known. International Publication No. 2017/048194 discloses a flexible robotic endoscopy system that causes a memory to store position information regarding an initial position for performing treatment such as endoscopic submucosal dissection (ESD) and that moves an inserted shaft based on the stored position information.

### SUMMARY

In accordance with one of some aspect, there is provided a medical manipulator system comprising:
an outer manipulator including an imager that captures an image;
an inner manipulator that protrudes from a distal end surface of the outer manipulator;
a driving device that controls the outer manipulator and the inner manipulator; and
a processor,
wherein the processor
acquires the image in which a treatment target is seen from the imager, recognizes a region of the treatment target from the image,
acquires three-dimensional shape information regarding a lumen,
acquires three-dimensional shape information regarding the outer manipulator,
calculates information regarding a work region that is an operable range of the inner manipulator with respect to each of a plurality of positions in a movable range of the outer manipulator to acquire information regarding a plurality of the work regions, and
performs drive control of the outer manipulator to move to a first position corresponding to a first work region selected from among the plurality of work regions based on the region of the treatment target, the three-dimensional shape information regarding the lumen, and the three-dimensional shape information regarding the outer manipulator.

In accordance with one of some aspect, there is provided an processor that controls an outer manipulator including an imager that captures an image, an inner manipulator that protrudes from a distal end surface of the outer manipulator, and a driving device that controls the outer manipulator and the inner manipulator,
wherein the processor
acquires the image in which a treatment target is seen from the imager,
recognizes a region of the treatment target from the image,
acquires three-dimensional shape information regarding a lumen,
acquires three-dimensional shape information regarding the outer manipulator,
calculates information regarding a work region that is an operable range of the inner manipulator with respect to each of a plurality of positions in a movable range of the outer manipulator to acquire information regarding a plurality of the work regions, and
performs drive control of the outer manipulator to move to a first position corresponding to a first work region selected from among the plurality of work regions based on the region of the treatment target, the three-dimensional shape information regarding the lumen, and the three-dimensional shape information regarding the outer manipulator.

In accordance with one of some aspect, there is provided an control method of controlling an outer manipulator including an imager that captures an image, an inner manipulator that protrudes from a distal end surface of the outer manipulator, and a driving device that controls the outer manipulator and the inner manipulator, the method comprising:
acquiring the image in which a treatment target is seen from the imager;
recognizing a region of the treatment target from the image;
acquiring three-dimensional shape information regarding a lumen;
acquiring three-dimensional shape information regarding the outer manipulator;
calculating information regarding a work region that is an operable range of the inner manipulator with respect to each of a plurality of positions in a movable range of the outer manipulator to acquire information regarding a plurality of the work regions; and
performing drive control of the outer manipulator to move to a first position corresponding to a first work region selected from among the plurality of work regions based on the region of the treatment target, the three-dimensional shape information regarding the lumen, and the three-dimensional shape information regarding the outer manipulator.

### BRIEF DESCRIPTION OF THE DRAWINGS

FIG. 1 is a block diagram for describing a configuration example of a medical manipulator system.

FIG. 2 is a diagram for describing a configuration example of a driving device.

FIG. 3 is a view for describing an example including three treatment tool channels.

FIG. 4 is another view for describing the example including the three treatment tool channels.

FIG. 5 is another view for describing the example including the three treatment tool channels.

FIG. 6 is a diagram for describing a configuration example of a second treatment tool driving device.

FIG. 7 is a view for describing a configuration example of a surgery system.

FIG. 8 is a view for describing a configuration example of a console.

FIG. 9 is a table for describing a driving device controlled by a foot pedal and a handle.

FIG. 10 is a view for describing a configuration example of the handle.

FIG. 11 is a table for describing an example of setting a control signal transmitted from the console.

FIG. 12 is a view for describing an example of a screen displayed on a display of the console.

FIG. 13 is a flowchart describing an example of the flow of treatment to which a method of the present embodiment is applied.

FIG. 14 is a flowchart describing a processing example according to the method of the present embodiment.

FIG. 15 is a flowchart describing a processing example associated with acquisition of three-dimensional information regarding a treatment target.

FIG. 16 is a flowchart describing a setting regarding positions of work regions and the order of the work regions.

FIG. 17 is a block diagram for describing another configuration example of an endoscope.

FIG. 18A is a view for describing an example of a second sensor. FIG. 18B is a view for describing another example of the second sensor. FIG. 18C is a view for describing an example of an optical fiber used in the second sensor.

FIG. 19 is a diagram for describing a reference for measuring position information regarding the treatment target.

FIG. 20 is a view for describing a relationship between a reference coordinate system and a distal end of the endoscope.

FIG. 21A is a view for describing position information regarding the treatment target and measured from a first sensor. FIG. 21B is a view for describing position information regarding the treatment target and measured from the distal end of the endoscope.

FIG. 22 is a view for describing a relationship between coordinates of the treatment target and coordinates of the distal end of the endoscope in an endoscope image.

FIG. 23 is a flowchart describing a processing example associated with calculation of a movable range of the distal end of the endoscope.

FIG. 24 is a view for describing a movable range of the endoscope.

FIGS. 25A and 25B are other views for describing the movable range of the endoscope.

FIG. 26 is another view for describing the movable range of the endoscope.

FIG. 27 is a flowchart describing a processing example regarding calculation of a first work region.

FIG. 28A is a view for describing a relationship between a first space region and an imaging range in an endoscope image. FIG. 28B is a view for describing an example of a predetermined range in the endoscope image.

FIG. 29 is a view for describing an example of calculation of the first work region.

FIGS. 30A and 30B are other views for describing an example of calculation of the first work region.

FIGS. 31A and 31B are views for describing examples of a relationship among an orientation of a distal end of an overtube, an orientation of the distal end of the endoscope, and an orientation along an inner wall of a lumen.

FIG. 32 is a view for describing an example of an angle between a direction of forward movement of a treatment tool and a direction of a distal end of a treatment tool.

FIGS. 33A and 33B are views for describing examples of an angle that is formed by the distal end of the treatment tool with respect to the treatment target.

FIGS. 34A and 34B are views for describing other examples of the relationship among the orientation of the distal end of the overtube, the orientation of the distal end of the endoscope, and the orientation along the inner wall of the lumen.

FIG. 35 is a view for describing an example of setting a first first work region group.

FIG. 36 is a flowchart describing an example of processing of setting a first second work region group.

FIG. 37 is a view for describing an example of setting the first second work region group.

FIGS. 38A to 38C are views for describing examples of a target in step S256.

FIG. 39 is a view for describing another example of the target in step S256.

FIG. 40 is a flowchart describing an example of processing of determining an operation mode.

FIG. 41 is a flowchart describing an example of processing of setting a first third work region group.

FIG. 42 is another flowchart describing an example of processing of setting the first third work region group.

FIG. 43 is a view for describing a method of setting the first third work region group.

FIG. 44 is another view for describing the method of setting the first third work region group.

FIG. 45 is another view for describing the method of setting the first third work region group.

FIG. 46 is a flowchart describing an example of processing of calculating a second work region.

FIG. 47 is a view for describing a method of calculating the second work region.

FIGS. 48A and 48B are other views for describing the method of calculating the second work region.

FIG. 49 is another view for describing the method of calculating the second work region.

FIG. 50 is another view for describing the method of calculating the second work region.

FIG. 51 is a flowchart describing an example of processing of setting a second second work region group.

FIG. 52 is a flowchart describing an example of processing of setting a second third work region group.

FIG. 53 is a view for describing an example of a method of setting the second third work region group.

FIG. 54 is another view for describing an example of the method of setting the second third work region group.

FIG. 55 is a view for describing another example of the method of setting the second third work region group.

FIG. 56 is another view for describing another example of the method of setting the second third work region group.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

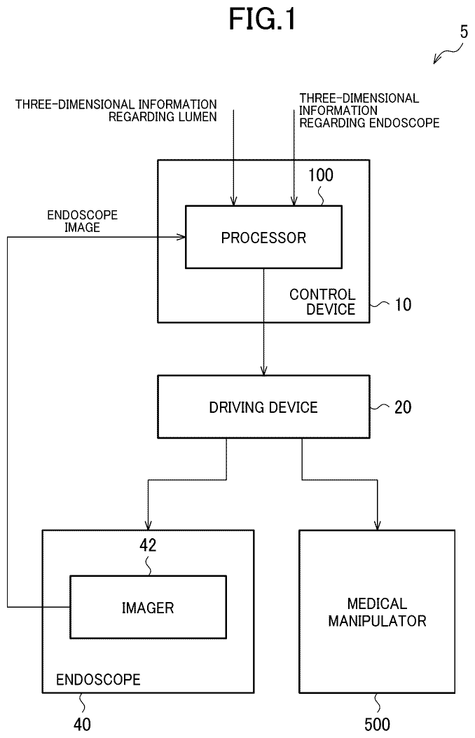

A configuration example of a medical manipulator system 5 in the present embodiment will be described with reference to FIG. 1. The medical manipulator system 5 in the present embodiment includes a control device 10, a driving device 20, and an endoscope 40 (an outer manipulator). The control device 10 includes a processor 100. The control device 10 in the present embodiment controls the driving device 20. Additionally, the driving device 20 in the present embodiment controls the endoscope 40 and a medical manipulator 500 (an inner manipulator). Note that, as described later, there may be a plurality of medical manipulators 500, which may be specifically referred to as a first medical manipulator 510, a second medical manipulator 520, a third medical manipulator 530, and the like, as necessary.

A method associated with the medical manipulator system 5 in the present embodiment is applicable to, for example, manipulation regarding ESD as described later, but is not prevented from being applied to other manipulation such as EMR. Additionally, part of the following method may be applied to another treatment. Note that ESD is an abbreviation for endoscopic submucosal dissection and EMR is an abbreviation for endoscopic mucosal resection. In the following description, the endoscope 40 in the present embodiment is exemplified by a medical flexible endoscope mainly used in ESD, but the method of the present embodiment is not prevented from being applied to another endoscope.

Additionally, since a component that is well-known as a component of the flexible endoscope is applicable to each component of the endoscope 40 in the present embodiment, a detailed illustration and description thereof will be omitted as appropriate in the following description. Examples of each component of the endoscope 40 include an insertion portion of the endoscope 40. The insertion portion includes a bending portion and a distal end portion. Specifically, the distal end portion of the endoscope includes an imager 42, and may further include a cap 48, Details of the imager 42 and the cap 48 will be described later. In the following description, the distal end portion of the endoscope 40 is simply referred to as an “endoscope distal end portion”, the insertion portion of the endoscope 40 is simply referred to as an “endoscope insertion portion”, and the bending portion of the endoscope 40 is simply referred to as an “endoscope bending portion”.

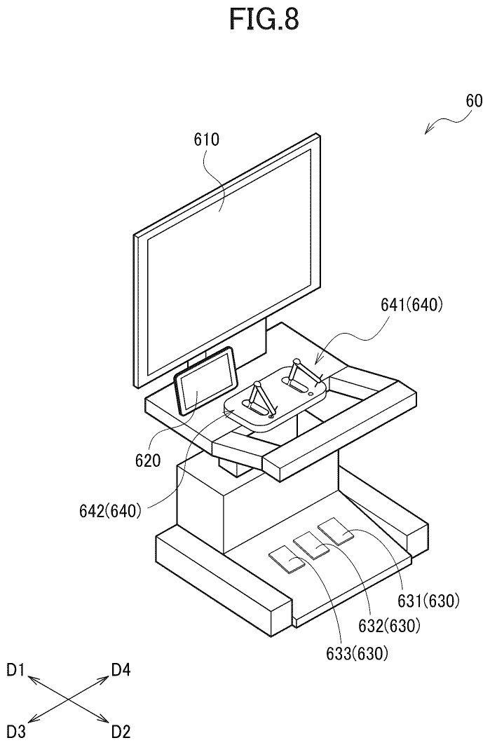

The endoscope 40 in the present embodiment includes the imager 42. The imager 42 includes an imaging sensor, an optical member, and the like, and functions as an imaging device. The imaging sensor includes a charge-coupled device (CCD), a complementary metal-oxide semiconductor (CMOS) sensor, or the like. In the present embodiment, an image captured by the imager 42 is an endoscope image. The imager 42 captures light that has been emitted from an illumination device and returned from a subject to form an image, and outputs an image signal to the processor 100 in the control device 10 via a cable or the like. The illumination device and the cable are not illustrated. The processor 100 generates a display image based on the image signal, and outputs the display image to, for example, a display 610 or the like. The display 610 will be described later with reference to FIG. 8 and the like. Note that the image signal includes a video signal, and the endoscope image may be a still image converted from a video image captured based on the video signal. Note that, as described later, there may be a plurality of treatment tools 50, which may be specifically referred to as a first treatment tool 51, a second treatment tool 52, a third treatment tool 53, and the like. Additionally, the illumination device, which is not illustrated, may have, for example, a plurality of illumination modes. For example, the illumination device includes a plurality of types of filters through which light with a desired wavelength is caused to pass by control of the illumination device. The control device 10 performs processing of selecting a filter as appropriate depending on a situation, and light that has passed through the filter is emitted to a subject. This allows a user to smoothly perform treatment. Note that, since multitudes of known methods regarding illumination modes have been proposed, details thereof are omitted.

The processor 100 in the present embodiment has the following hardware configuration. The hardware can include at least one of a circuit that processes a digital signal or a circuit that processes an analog signal. For example, the hardware can include one or more circuit devices mounted on a circuit substrate, or one or more circuit elements. The one or more circuit devices are, for example, integrated circuits (ICs) or the like. The one or more circuit elements are, for example, resistors, capacitors, or the like.

Additionally, for example, the processor 100 in the present embodiment is also capable of operating based on a memory that is not illustrated (hereinafter simply referred to as a “memory”) and information stored in the memory. The information is, for example, a program, various kinds of data, or the like. A central processing unit (CPU), a graphics processing unit (GPU), a digital signal processor (DSP), or the like can be used as the processor 100. The memory may be a semiconductor memory such as a static random-access memory (SRAM) and a dynamic random access memory (DRAM). The memory may be a register. The memory may be a magnetic storage device such as a hard disk device. The memory may be an optical storage device such as an optical disk device. For example, the memory stores a computer-readable instruction. The instruction is executed by the processor 100, whereby a function of each section of the control device 10 is implemented as processing. The instruction mentioned herein may be an instruction set that is included in the program, or may be an instruction that instructs a hardware circuit included in the processor 100 to operate. Additionally, the memory is also referred to as a storage device.

Furthermore, in the present embodiment, the processor 100 acquires three-dimensional shape information regarding a lumen and three-dimensional shape information regarding the endoscope 40. A specific method of acquiring these pieces of information will be described later.

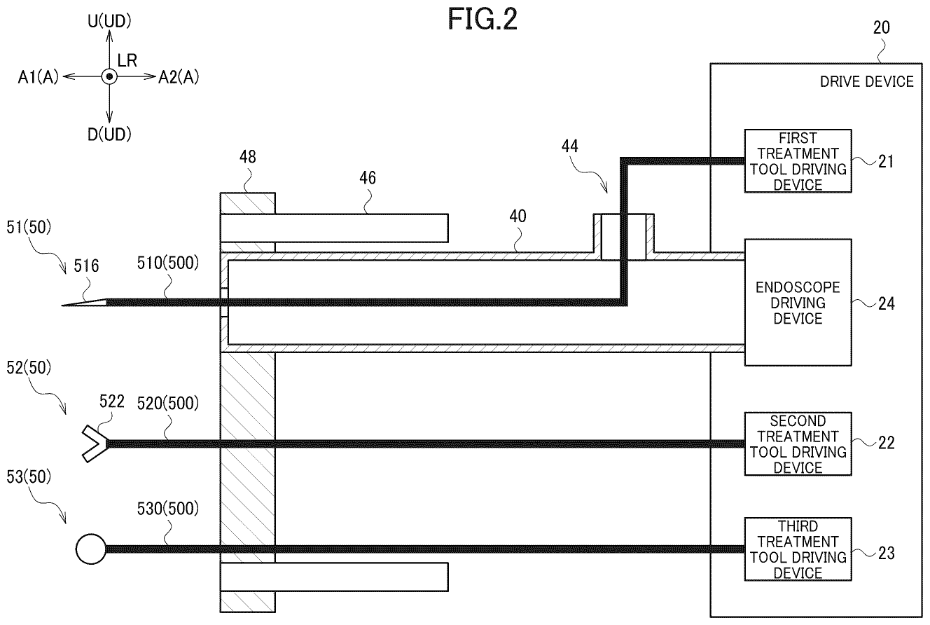

The driving device 20 in the present embodiment is now described. The driving device 20 in the present embodiment can be configured, for example, as illustrated in FIG. 2. The driving device 20 includes a first treatment tool driving device 21 that controls each portion of the first treatment tool 51, a second treatment tool driving device 22 that controls each portion of the second treatment tool 52, a third treatment tool driving device 23 that controls each portion of the third treatment tool 53, and an endoscope driving device 24 that controls each portion of the endoscope 40. Note that FIG. 2 is a conceptual diagram, and illustration of part of components such as the imager 42 is omitted for explanatory convenience.

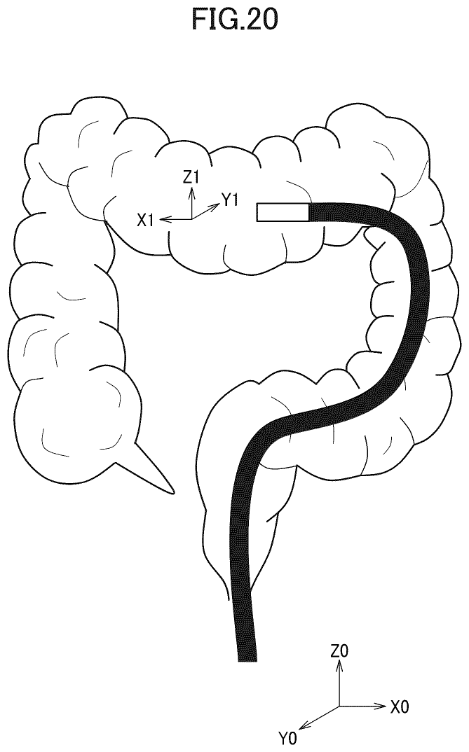

Additionally, an A axis, a UD axis, and an LR axis as three-dimensional coordinates in a right-handed system are illustrated as appropriate in FIGS. 2 to 6. A direction of the A axis is a direction in parallel with a direction along a longitudinal direction of the medical manipulator 500 using the endoscope distal end portion as a reference. Assume that a direction of forward movement of the medical manipulator 500 is an A1 direction, and a direction of backward movement of the medical manipulator 500 is an A2 direction. Forward movement and backward movement may be hereinafter simply referred to as “forward/backward movement. In other words, the direction of the A axis is a direction along a direction of the forward/backward movement of the medical manipulator 500. Additionally, a direction along the UD axis is referred to as a UD axis direction, and a direction along the LR axis is referred to as an LR axis direction. Note that coordinate axes in FIGS. 2 to 6 are illustrated for convenience for describing the driving device 20, and do not necessarily correspond to coordinate axes illustrated in FIG. 20 or subsequent drawings.

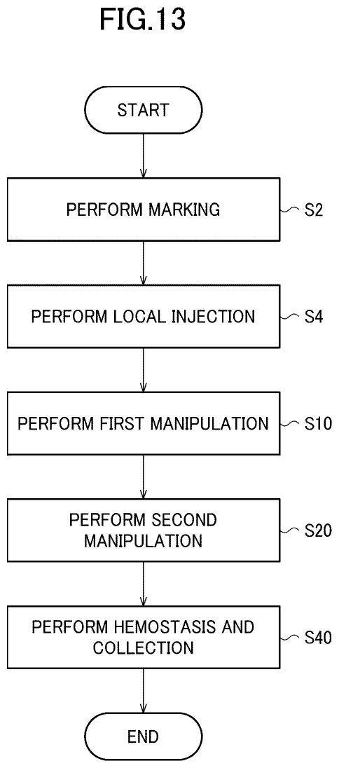

The first treatment tool 51 is, for example, a local injection needle. In this case, the first treatment tool 51 includes, for example, the first medical manipulator 510 and an injection needle 516 located at a distal end of the first medical manipulator 510. The injection needle 516 is used, for example, for injecting a local injection filled in a syringe, which is not illustrated, into a submucosal layer. The local injection is, for example, normal saline or the like, and may contain a predetermined ratio of sodium hyaluronate and the like. This makes it possible to increase viscosity of the local injection. Accordingly, it is possible to maintain a state where a mucosa is raised high. Eventually, it is possible to minimize damage on a muscle layer in first manipulation (step S10) and second manipulation (step S20), which will be described later with reference to FIG. 13 or subsequent drawings. As a result, it is possible to lower possibility of occurrence of a complication associated with damage to the muscle layer. Additionally, the local injection may contain a pigment substance. The predetermined pigment substance is, for example, indigocarmine or the like. Accordingly, the submucosal layer on which local injection has been performed becomes blue-tinged transparent, allowing the user to appropriately set a treatment target region, which will be described later.

The second treatment tool 52 is, for example, grasping forceps. In this case, the second treatment tool 52 includes, for example, the second medical manipulator 520 and a grasping portion 522 located at a distal end of the second medical manipulator 520.

Additionally, the third treatment tool 53 is, for example, a high-frequency knife. In this case, the third treatment tool 53 includes, for example, the third medical manipulator 530. The third treatment tool 53 has a configuration that allows a knife portion to protrude from the distal end of the third medical manipulator 530, as necessary. The knife portion includes a wire for power supply, a high-frequency electrode, and the like. The wire for power supply and the high-frequency electrode are not illustrated. For example, high-frequency current is supplied to the high-frequency electrode from a power supply device, which is not illustrated, via the wire for supplying power to energize the high-frequency electrode. By bringing the high-frequency electrode into contact with a desired biological tissue in this state, the biological tissue is cauterized by heat energy generated from the high-frequency electrode. Marking, incision, hemostasis, or the like is performed by control of the heat energy or the like. The marking will be described later. Note that the shape of the knife portion illustrated in FIG. 2 and the like is exemplified by a pole-type, but is not limited thereto and may be a scalpel-type, a needle-type, a hook-type, a scissor-type, a tweezer-type, or the like. That is, a wide range of configurations associated with known high-frequency treatment tools are applicable to the distal end of the third treatment tool 53 in the present embodiment.

As illustrated in FIG. 2, the medical manipulator system 5 in the present embodiment is controlled by electric power. More specifically, all of the second treatment tool 52, the third treatment tool 53, and the endoscope 40 are controlled by electric power. Note that, in the present embodiment, the first treatment tool 51 need not be necessarily controlled by electric power, but may be further controlled by electric power. Additionally, an operation by electric power mentioned herein means that the medical manipulator 500 is driven by an actuator such as a motor based on an electric signal for controlling the operation of the medical manipulator 500 or the like. Additionally, driving of the medical manipulator 500 or the like by electric power includes, other than driving of the medical manipulator 500 or the like based on determination made by the processor 100, driving of the medical manipulator 500 or the like by electric power in response to the user's manual operation of a console 60, which will be described later.

Controlling the medical manipulator system 5 by electric power in this manner implements manipulation that will be described later. Part of steps associated with first manipulation (step S10) and second manipulation (step S20) can be performed by automatic control of the endoscope 40. The first manipulation and the second manipulation will be described later. The automatic control of the endoscope 40 mentioned herein means that the processor 100, instead of the user, makes determination to control each portion included in the endoscope 40 and the like, That is, the processor 100 uses a predetermined control algorithm to control each portion included in the endoscope 40 and the like.

In this manner, in the medical manipulator system 5 in the present embodiment, the first treatment tool 51, the second treatment tool 52, and the third treatment tool 53 are, together with the endoscope 40, inserted into the inside of the body, and each treatment associated with ESD is performed. However, the endoscope 40 need not have three treatment tool channels. For example, the medical manipulator system 5 in the present embodiment may further include an overtube 46. Alternatively, a configuration in which the overtube 46 includes a plurality of treatment tool channels may be adopted. For example, as illustrated in FIG. 2, the endoscope 40 includes one treatment tool insertion opening 44, the first treatment tool 51 is inserted into the treatment tool insertion opening 44, and the second treatment tool 52 and the third treatment tool 53 are inserted into respective two treatment tool channels in the overtube 46.

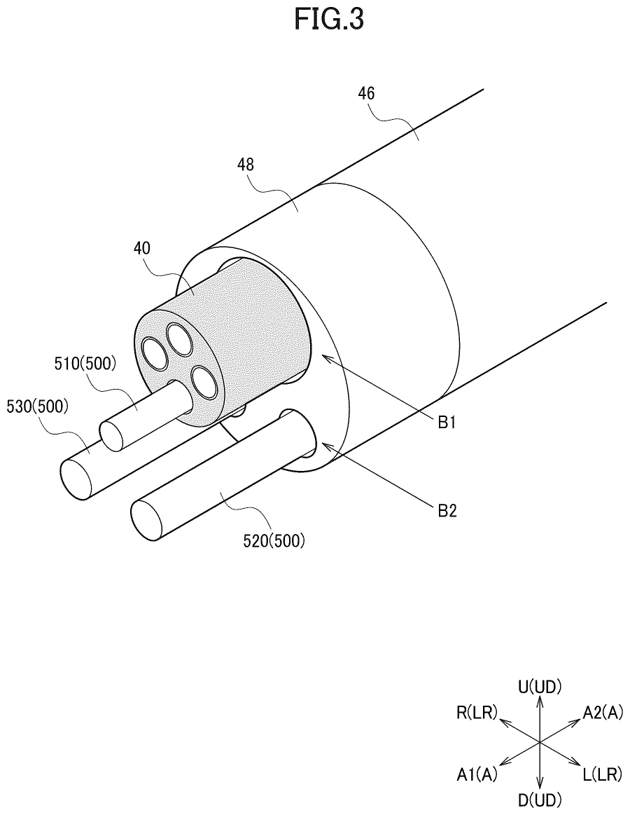

Additionally, as illustrated in FIG. 2, the medical manipulator system 5 in the present embodiment may further include a cap 48. While various kinds of known shapes of the cap 48 have been proposed, the cap 48 includes an endoscope exit hole indicated by B1 and treatment tool exit holes indicated by B2 as illustrated in FIG. 3, for example. In the cap 48 illustrated in FIG. 3, one endoscope exit hole and two treatment tool exit holes are formed so as to correspond to the example illustrated in FIG. 2. That is, as illustrated in FIG. 3, the first medical manipulator 510 protrudes from the endoscope exit hole indicated by B1, and the second medical manipulator 520 and the third medical manipulator 530 protrude from the respective treatment tool exit holes indicated by B2. Note that illustration is given so that the distal end of the endoscope 40 protrudes from the endoscope exit hole indicated by B1 for explanatory convenience, but the cap 48 may have a configuration in which the distal end of the endoscope 40 does not protrude. Additionally, although illustration is omitted for convenience, a tube that forms a channel may be further included between each hole in the cap 48 and the driving device 20. This makes it possible to smoothly perform an operation of the medical manipulator 500.

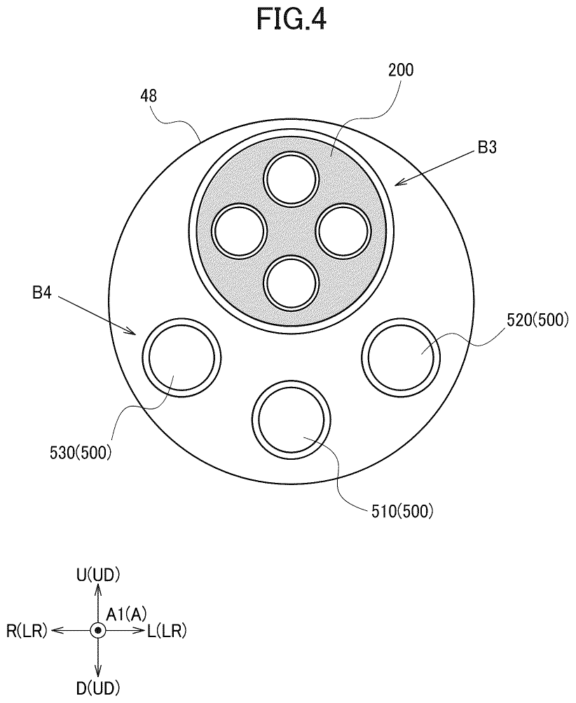

Additionally, in FIGS. 2 and 3, one treatment tool 50 protrudes from the cap 48 via the insertion portion of the endoscope 40 and two treatment tools 50 protrude from the cap 48 via the overtube 46, but the configuration is not limited thereto. For example, as illustrated in FIG. 4, treatment according to the method of the present embodiment may be performed with the three treatment tools 50 protruding from the cap 48 via the overtube 46. In the cap 48 illustrated in FIG. 4, one endoscope exit hole indicated by B3 is formed, and three treatment tool exit holes indicated by B4 are formed. Alternatively, although illustration is omitted, for example, a configuration may be adopted in which the endoscope 40 includes two treatment tool channels, two treatment tools 50 protrude from the cap 48 via the endoscope insertion portion, and one treatment tool 50 protrudes from the cap 48 via the overtube 46. Still alternatively, although illustration is omitted, an example in which the three treatment tools 50 protrude from the cap 48 via the endoscope insertion portion may be adopted.

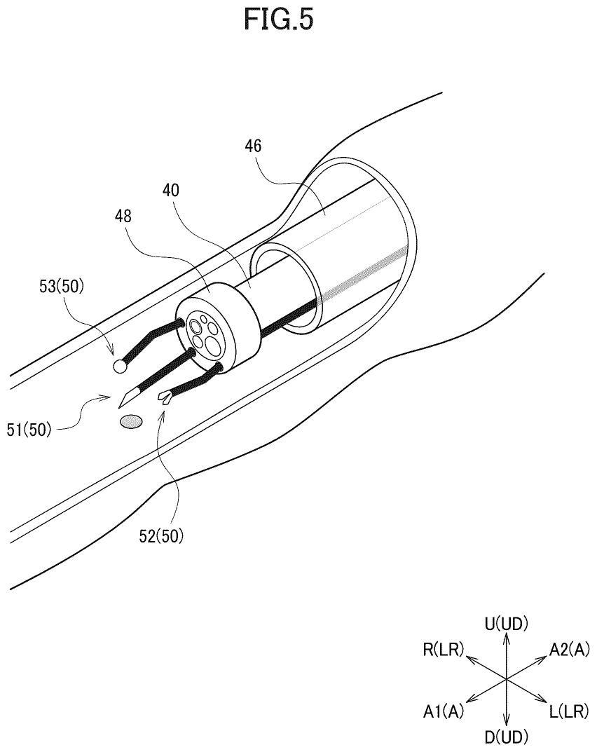

Note that FIGS. 2 and 3 give illustration so that the cap 48 is fitted to and integrated with the overtube 46, but, for example, the cap 48 may be fitted to the endoscope 40 so as to allow the endoscope 40 to be displaced independently of the overtube 46, as illustrated in FIG. 5. For example, a configuration may be adopted in which, at the time of start of treatment, the endoscope 40, the overtube 46, and the cap 48 are inserted into the inside of the body in an integrated state, the overtube 46 is fixed with a balloon when the endoscope distal end portion approaches a location of the treatment target, and the endoscope 40 to which the cap 48 is fitted is driven separately from the overtube 46. Additionally, FIG. 2 and the like give illustration so that the cap 48 has a size that is identical to or more than a size of the overtube 46, but, for example, may have a size that is smaller than the size of the overtube 46. This allows the endoscope 40 that is fitted to the cap 48 to drive the inside of the overtube 46.

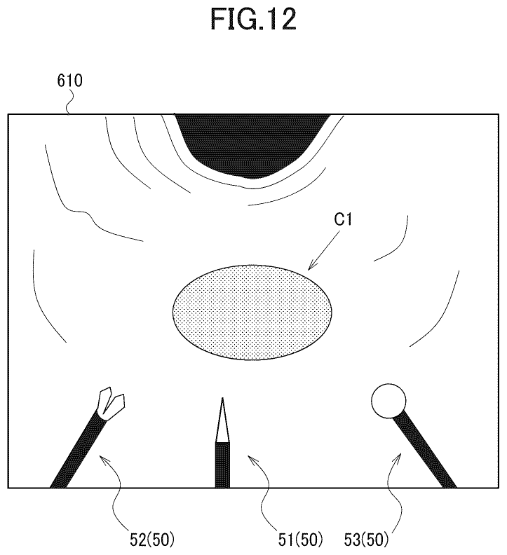

In this manner, in a case where the endoscope distal end portion includes the cap 48, it is possible to make a relationship between the position of the endoscope 40 and the position of each medical manipulator 500 correspond to a relationship between the position of the endoscope exit hole formed in the cap 48 and the position of each treatment tool exit hole in a plan view when viewed from a direction along the A axis. Additionally, for example, a plurality of types of caps 48 that is different in relationship between the position of the endoscope exit hole and the position of each treatment tool exit hole may be prepared to allow the user to select an appropriate cap 48 depending on treatment. This configuration changes a positional relationship between the imager 42 and each medical manipulator 500, which makes it possible to change the position at which each treatment tool 50 can be visually recognized in an endoscope image, whereby it is possible to acquire an endoscope image appropriate for the treatment. For example, in a case where it is possible to perform display illustrated in FIG. 12, which will be described later, it is sufficient if the cap 48 illustrated in FIG. 5 is selected. However, there is an assumed case where the display as illustrated in FIG. 12 cannot be necessarily displayed due to a position at which the treatment target exists or other reasons. In such a case, the user selects a different type of the cap 48, which makes it possible to change a position where the treatment tool 50 can be seen in the endoscope image. As a result, it is possible to perform the treatment smoothly.

Alternatively, although illustration is omitted, for example, a configuration example may be adopted in which the medical manipulator system 5 in the present embodiment does not include the cap 48, but includes the overtube 46 including a plurality of tubes forming an endoscope insertion portion channel and a plurality of treatment tool channels. In this case, a relationship between the position of a tube forming the endoscope channel and the position of a tube forming each treatment tool channel corresponds to a relationship between the position of the endoscope 40 and the position of each medical manipulator 500 in the plan view when viewed from the direction along the A axis. Additionally, a plurality of types of overtubes 46 that is different in relationship between the position of the endoscope insertion portion channel and the position of each of the plurality of treatment tool channels may be prepared to allow the user to select an appropriate overtube 46 depending on treatment. With this configuration, it is possible to obtain an effect that is similar to an effect obtained in a case where the plurality of types of caps 48 is selectable.

Additionally, although not illustrated in FIG. 2 and the like, for example, the driving device 20 may further include an overtube driving device that drives the overtube 46. A driving device that is equivalent to the endoscope driving device 24 can constitute the overtube driving device.

The endoscope 40 and the treatment tool 50 in the present embodiment include driving units, the number of which depends on a required degree of freedom. For example, the second treatment tool driving device 22 includes a motor unit 220. The motor unit 220 includes, for example, a first bending motion driving section 221, a second bending motion driving section 222, an opening/closing operation driving section 223, a rolling operation driving section 224, and a forward/backward movement operation driving section 225.

The first bending motion driving section 221 pulls or loosens a pair of wires, which is not illustrated, based on a control signal received from the control device 10, and thereby bends the second medical manipulator 520 in a direction along the UD axis. As a result, the orientation of the grasping portion 522 changes along a direction indicated by D21. Similarly, the second bending motion driving section 222 bends the second medical manipulator 520 in a direction along the LR axis based on a control signal received from the control device 10. As a result, the orientation of the grasping portion 522 changes along a direction indicated by D22.

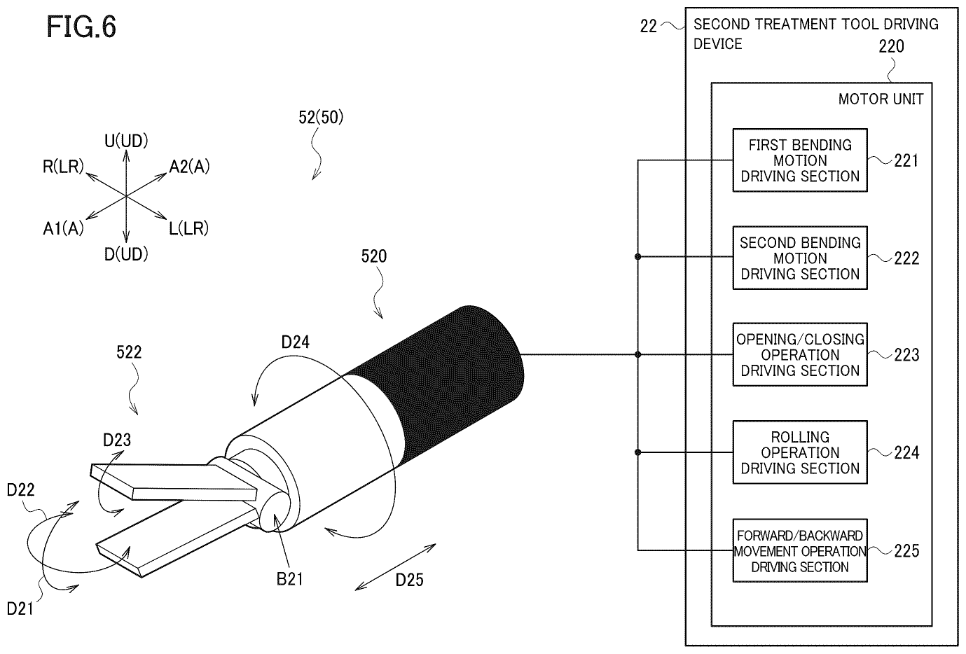

The opening/closing operation driving section 223 controls an opening/closing operation of the grasping portion 522. For example, one of grasping pieces pivots about a pivot shaft indicated by B21 along a direction indicated by D23 based on a control signal received from the control device 10. Note that the grasping portion 522 illustrated in FIG. 6 is merely an example, and a wide range of known structures can be applied to the grasping portion 522.

The rolling operation driving section 224 controls a rolling operation of the distal end portion of the second medical manipulator 520. For example, the rolling operation driving section 224 rolls the distal end portion of the second medical manipulator 520 along a direction indicated by D24 based on a control signal received from the control device 10.

The forward/backward movement operation driving section 225 controls a forward/backward movement operation of the distal end portion of the second medical manipulator 520. The forward/backward movement operation driving section 225 uses, for example, a driving mechanism including a direct-acting motor to move the second medical manipulator 520 forward/backward based on a control signal received from the control device 10.

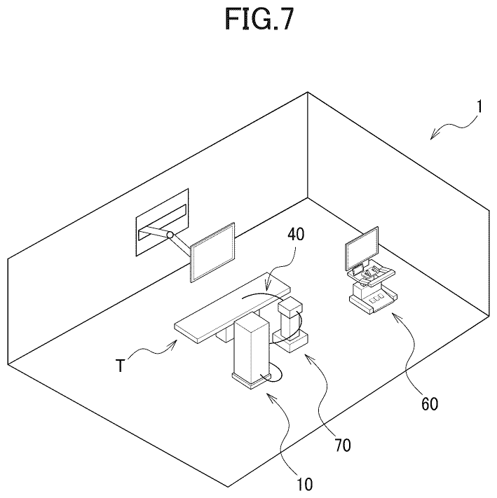

Note that a driving unit in the third treatment tool driving device 23 and a driving unit in the endoscope driving device 24 can be implemented by a driving unit that is similar to the above-mentioned motor unit 220 in the second treatment tool driving device 22. Note that, in the third treatment tool driving device 23, for example, a configuration corresponding to the above-mentioned rolling operation driving section 224 may be omitted. Additionally, in the endoscope driving device 24, for example, a driving unit 70, which will be described later with reference to FIG. 7, may slide in a predetermined direction with respect to the floor to implement a function that is equivalent to that of the above-mentioned forward/backward movement operation driving section 225.

Additionally, the method of the present embodiment may be implemented as a surgery system 1 including the medical manipulator system 5. The surgery system 1 further includes, in addition to the control device 10 and the endoscope 40 described above, a console 60 and the driving unit 70 as illustrated in FIG. 7. The console 60 establishes wireless communication connection with the control device 10 using a communication method in conformity with a wireless communication standard such as Wireless Fidelity (Wi-Fi) (registered trademark), but may establish wired communication connection. In the surgery system 1 in the present embodiment, the endoscope 40 is inserted into the inside of the body of a subject that lies on an operating table TA, and treatment or the like is performed. The subject is not illustrated. The treatment will be described later with FIG. 13 and the like.

The driving unit 70 corresponds to the endoscope driving device 24 in FIG. 2 and operates each portion of the endoscope 40 by electric power based on a control signal from the control device 10. Note that, although not illustrated, units corresponding to the first treatment tool driving device 21, the second treatment tool driving device 22, and the third treatment tool driving device 23 may be included in the driving unit 70, or may be provided separately from the driving unit 70. The determination is only required to be made by the user as appropriate.

The console 60 includes, for example, a display 610, a touch panel 620, a foot pedal 630, and a handle 640. The display 610 displays an endoscope image captured by the imager 42 via the control device 10. The touch panel 620 displays an endoscope image similarly to the display 610, and also includes functions such as drawing. Additionally, the touch panel 620 may display, for example, a partial region including a middle of the display 610. Note that there may be a plurality of foot pedals 630 and a plurality of handles 640. For example, as illustrated in FIG. 8, the console 60 in the present embodiment includes, as the foot pedals 630, a first foot pedal 631, a second foot pedal 632, and a third foot pedal 633. Note that there may be four or more foot pedals 630. Similarly, the console 60 in the present embodiment includes, as the handles 640, a first handle 641 and a second handle 642. This allows the user to use the two handles 640 to perform treatment while using the endoscope 40 and the plurality of treatment tools 50 differently as appropriate. Note that, although illustration is omitted, the console 60 may further include, in addition to the handle 640, an operation section for performing other operations. Examples of the other operations include an air supply/water supply operation and an operation of changing an illumination mode of a light source device.

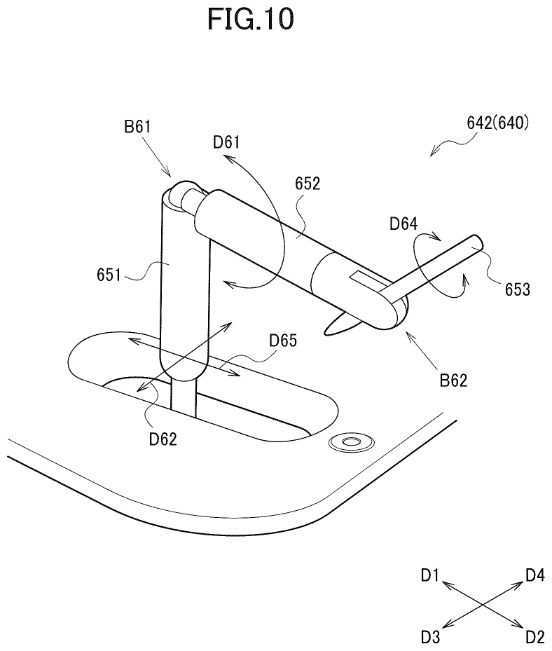

Note that a direction indicated by D1 in FIG. 8 is a direction along a direction in which the user faces the console 60, and is also referred to as a front direction. A direction indicated by D2 is the opposite direction of the direction indicated by D1, and is also referred to as a back direction. The direction indicated by D1 and the direction indicated by D2 are collectively referred to as a front-back direction. A direction indicated by D3 is orthogonal to the front-back direction, and is also referred to as a left direction. A direction indicated by D4 is the opposite direction of the direction indicated by D3, and is also referred to as a right direction. The direction indicated by D3 and the direction indicated by D4 are collectively referred to as a right-left direction. The same applies to FIG. 10, which will be described later.

In the console 60 in the present embodiment, the foot pedal 630 includes a foot switch, which is not illustrated, and a control signal is transmitted from the foot switch to the control device 10 by the user's operation of stepping on the foot pedal 630. The control device 10 controls the endoscope 40 or the medical manipulator 500 according to a combination of a control signal based on an operation of the foot pedal 630 and a control signal based on an operation of the handle 640, which will be described later.

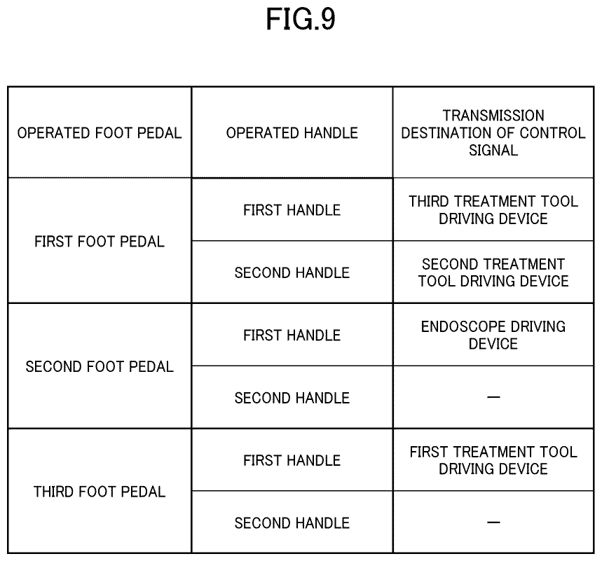

More specifically, for example, a table illustrated in FIG. 9 is stored in the memory included in the control device 10. For example, in a case where the user operates the first foot pedal 631 and operates the first handle 641, the control device 10 transmits a control signal from the first handle 641 to the third treatment tool driving device 23. Additionally, in a case where the user operates the first foot pedal 631 and operates the second handle 642, the control device 10 transmits a control signal from the second handle 642 to the second treatment tool driving device 22. Additionally, in a case where the user operates the second foot pedal 632 and operates the first handle 641, the control device 10 transmits a control signal from the first handle 641 to the endoscope driving device 24. Additionally, in a case where the user operates the third foot pedal 633 and operates the first handle 641, the control device 10 transmits a control signal from the first handle 641 to the first treatment tool driving device 21.

The memory included in the control device 10 stores the table illustrated in FIG. 9, which allows the user to operate the first handle 641 on the right side while stepping on the second foot pedal 632 at a middle of the console 60 to operate the endoscope 40. Additionally, it is possible for the user to operate the first handle 641 on the right side while stepping on the third foot pedal 633 on the left side of the console 60 to operate the first treatment tool 51. Furthermore, it is possible for the user to operate the second handle 642 on the left side while stepping on the first foot pedal 631 on the right side of the console 60 to operate the second treatment tool 52, and it is possible for the user to operate the first handle 641 on the right side to operate the third treatment tool 53.

More specifically, the handle 640 may be configured as illustrated in FIG. 10. In FIG. 10, the handle 640 includes a first part 651, a second part 652, and a third part 653. The first part 651 and the second part 652 are connected to each other via a joint indicated by B61. The second part 652 and the third part 653 are connected to each other via a joint indicated by B62. Note that FIG. 10 illustrates only the second handle 642, but the first handle 641 is similar to the second handle 642.

The first part 651 is capable of being displaced in a direction indicated by D62 and a direction indicated by D65. The direction indicated by D62 is identical to the right-left direction described above with reference to FIG. 8. The direction indicated by D65 is identical to the front-back direction described above with reference to FIG. 8. For example, the user moves the second part 652 along the direction indicated by D65 in a state of holding the second part 652, whereby the first part 651 is displaced in the front-back direction via the joint indicated by B61. Similarly, for example, the user moves the second part 652 along the direction indicated by D62 in a state of holding the second part 652, whereby the first part 651 is displaced in the right-left direction via the joint indicated by B61. Additionally, it is possible for the user to cause the second part 652 to pivot about a pivot shaft provided in the joint indicated by B61 in the direction indicated by D61. Furthermore, it is possible for the user to roll the third part 653 in a direction indicated by D64 about an axis in a longitudinal direction of the third part 653.

The user operates the handle 640 to perform forward/backward movement, bending, rolling, or the like of the medical manipulator 500 based on a control signal output from a sensor, which is not illustrated. For example, assume that the user wants to operate the second treatment tool 52 by operating the second handle 642 while stepping on the first foot pedal 631 as described above. For example, the user causes the second part 652 to pivot in the direction indicated by D61 in FIG. 10, and thereby bends the second medical manipulator 520 in the direction indicated by D21 in FIG. 6. Additionally, for example, the user operates the second handle 642 to displace the first part 651 in the direction indicated by D62 in FIG. 10, and thereby bends the second medical manipulator 520 in the direction indicated by D22 in FIG. 6. Additionally, for example, the user causes the third part 653 to roll in the direction indicated by D64 in FIG. 10, and thereby causes the second medical manipulator 520 to roll in the direction indicated by D24 in FIG. 6. Additionally, for example, the user operates the second handle 642 to displace the first part 651 in the direction indicated by D65 in FIG. 10, and thereby moves the second medical manipulator 520 forward/backward in the direction indicated by D25 in FIG. 6.

Although not illustrated, the handle 640 may further include an operation section such as a button. For example, high-frequency current may be caused to flow to the high-frequency electrode in the third treatment tool 53 by the user's operation of a button included in the first handle 641 in a state of stepping on the first foot pedal 631. Additionally, for example, the opening/closing of the grasping portion 522 of the second treatment tool 52 may be controllable by the user's operation of a button included in the second handle 642 while stepping on the first foot pedal 631.

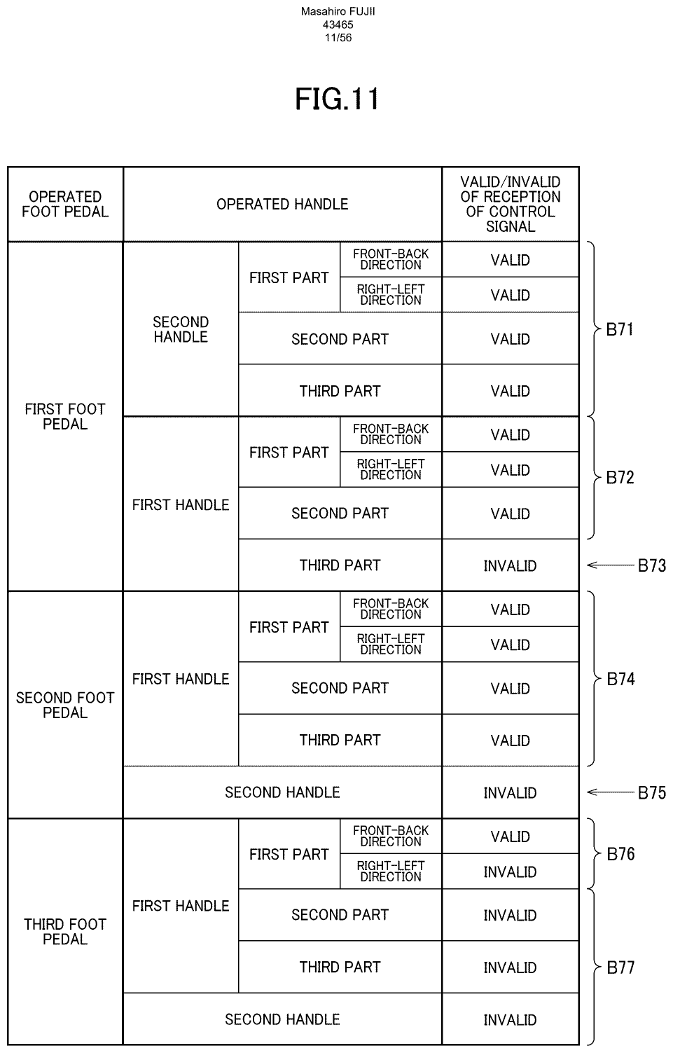

Additionally, for example, the control device 10 may be capable of setting whether to enable or disable reception of a control signal transmitted from the handle 640. To enable the reception of the control signal transmitted from the handle 640 means to transmit a corresponding control signal to each section of the driving device 20 based on the control signal received from the handle 640. To disable the reception of the control signal transmitted from the handle 640 means to discard an instruction based on the control signal received from the handle 640. Specifically, for example, in addition to the above-mentioned table illustrated in FIG. 9, a table illustrated in FIG. 11 is stored in the memory in the control device 10. For example, since the second medical manipulator 520 performs any of the forward/backward movement, bending, and rolling as described above with reference to FIG. 6, the reception of a control signal based on an operation of any of the first part 651, the second part 652, and the third part 653 is set to be valid as indicated by B71 in FIG. 11.

There is a case where a specification defines that the third medical manipulator 530 performs forward/backward movement and bending, but does not perform rolling. In this case, the reception of control signals based on the operation of the first part 651 and the operation of the second part 652 is set to be valid as indicated by B72 in FIG. 11, but the reception of a control signal based on the operation of the third part 653 is set to be invalid as indicated by B73 in FIG. 11.

Additionally, for example, since the endoscope 40 performs any of the forward/backward movement, bending, and rolling as described above, the reception of a control signal based on an operation of any of the first part 651, the second part 652, and the third part 653 is set to be valid as indicated by B74 in FIG. 11. Additionally, according to the table in FIG. 9, when the user steps on the second foot pedal 632, a driving mechanism as a transmission destination of the control signal based on the operation of the second handle 642 is not set, the reception of the control signal based on the operation of the second handle 642 is set to be invalid as indicated by B75 in FIG. 11.

Furthermore, for example, there is a case where the first treatment tool driving device 21 is configured to perform only forward/backward movement of the first treatment tool 51. Additionally, when the user steps on the third foot pedal 633, a driving mechanism as a transmission destination of the control signal based on the operation of the second handle 642 is not set. In this case, as indicated by B76 in FIG. 11, the reception of the control signal based on the operation of the first part 651 in the front-back direction is set to be valid, but the reception of the control signal based on the operation of the first part 651 in the right-left direction is set to be invalid. Additionally, as indicated by B77 in FIG. 11, the reception of other control signals is set to be invalid.

Note that a combination of the operation of the foot pedal 630 and the operation of the handle 640 is not limited to the above combinations. For example, in a case where the driving device 20 further includes the above-mentioned overtube driving device, a breakdown of the table in FIG. 9 may be changed as follows. For example, in a case where the user operates the second foot pedal 632 and operates the second handle 642, the control device 10 may transmit the control signal from the second handle 642 to the first treatment tool driving device 21. Additionally, for example, in a case where the user operates the third foot pedal 633 and operates the first handle 641, the control device 10 may transmit a control signal from the first handle 641 to the overtube driving device.

With the console 60 configured in this manner, the user operates the treatment tool 50 as appropriate while watching the display 610, for example, as illustrated in FIG. 12, and performs treatment on a treatment target indicated by C1.

The treatment target in the present embodiment is a lesion, but a region including the lesion and further including a predetermined margin may be regarded as the treatment target. The lesion mentioned herein is a portion that is thought to be in a state different in appearance from a normal state, and is not necessarily limited to a portion that is attributed to a disease. The lesion is, for example, a tumor, but is not limited thereto, and may be a polyp, inflammation, diverticulum, or the like. The predetermined margin represents a region between a region in which marking is performed (step S2) and a region associated with the lesion, but may be a region that has been elevated by local injection (step S4) other than the lesion. The marking and the local injection will be described later with reference to FIG.13. Note that the treatment target associated with the method of the present embodiment is early cancer. The early cancer mentioned herein is a tumor that grows in a range within a mucosa layer or a submucosal layer.

Note that the description is given while the treatment target is illustrated as appropriate in the present embodiment, but illustration of a portion in which a tissue is changed due to the marking (step S2) and the local injection (step S4) is omitted for easier understanding of the gist of the present embodiment unless otherwise specified.

Additionally, the display in FIG. 12 is merely an example, and the first treatment tool 51, the second treatment tool 52, and the third treatment tool 53 need not be simultaneously displayed on the display 610. However, in a case where the method of the present embodiment is applied to the second manipulation (step S20), which will be described later, it is preferable that the second treatment tool 52 and the third treatment tool 53 can be simultaneously displayed.

The medical manipulator system 5 in the present embodiment can be applied to, for example, treatment described in a flowchart in FIG. 13. For example, the user performs marking (step S2). The marking (step S2) is a step of forming a predetermined mark for performing the first manipulation (step S10) or the second manipulation (step S20), which will be described later, in the periphery of a lesional region within a lumen. Note that a method of slightly cauterizing the surface of the tissue using the third treatment tool 53 is used to form the predetermined mark. The third treatment tool 53 will be described later. Alternatively, for example, the marking (step S2) may be performed by segmentation of the region associated with the lesion on a captured endoscope image.

Thereafter, the user performs local injection (step S4). Specifically, for example, the user injects a local injection into the submucosal layer so as to raise a portion on which the marking (step S2) has been performed. The local injection (step S4) is performed with the above-mentioned first treatment tool 51.

Thereafter, the user performs the first manipulation (step S10). The first manipulation (step S10) is, for example, peripheral incision. Specifically, for example, the user uses the third treatment tool 53 to incise the submucosal layer raised by the local injection (step S4) along a direction in which marks formed by the marking (step S2) are aligned. This makes it possible to minimize damage on a muscle layer.

Thereafter, the user performs the second manipulation (step S20). The second manipulation (step S20) is, for example, ESD. Note that ESD is an abbreviation for endoscopic submucosal dissection. The above-mentioned first manipulation (step S10) causes an outer peripheral portion of the lesion to be in a state of being exfoliated from a wall of the lumen, but the inside of the lesion is not limited to being exfoliated from the wall of the lumen. Then, for example, the user further performs incision with the third treatment tool 53 while grasping the lesion with the second treatment tool 52 so as to exfoliate the inside of the lesional region from the wall of the lesion. Note that, although not described in the flowchart, the user may further perform manipulation that is similar to the local injection (step S4) as necessary before starting the second manipulation (step S20). This makes it possible to bring the whole of the lesion into a state of floating from the submucosal layer, and thereby makes it possible to perform the second manipulation (step S20) more appropriately. Note that, for example, in a case where the lesion has a small size, the user may omit the first manipulation (step S10) and perform only the second manipulation (step S20) to perform the treatment in FIG. 13.

Note that, in a case where a description that is applied to both the first manipulation (step S10) and the second manipulation (step S20) is given below, the first manipulation (step S10) and the second manipulation (step S20) may be collectively simply referred to as “manipulation”.

Thereafter, the user performs hemostasis and collection (step S40). For example, the user uses the third treatment tool 53 to cauterize a location that bleeds as a result of the first manipulation (step S10) or the second manipulation (step S20) described above. Additionally, for example, the user moves the second treatment tool 52 backward to a position outside the body in a state of grasping the lesion with the grasping portion 522 of the second treatment tool 52 to collect the lesion. Alternatively, the user may replace any one of the first treatment tool 51, the second treatment tool 52, and the third treatment tool 53 with a dedicated treatment tool that collects the lesion such as a net treatment tool to collect the lesion. Furthermore, in a case where the lesion has a size that is larger than a diameter of the channel of the second medical manipulator 520, the user may move the endoscope 40 and the medical manipulator 500 backward to a position outside the body in a state of grasping the lesion with the grasping portion 522.

In the following description, the first manipulation (step S10) is exemplified by the peripheral incision, and the second manipulation (step S20) is exemplified by the exfoliation of the submucosal layer, but manipulation to which the present embodiment is applicable is not limited thereto. For example, all or part of the following method may be applied to the marking (step S2), the local injection (step S4) and the like, or may be applied to another treatment.

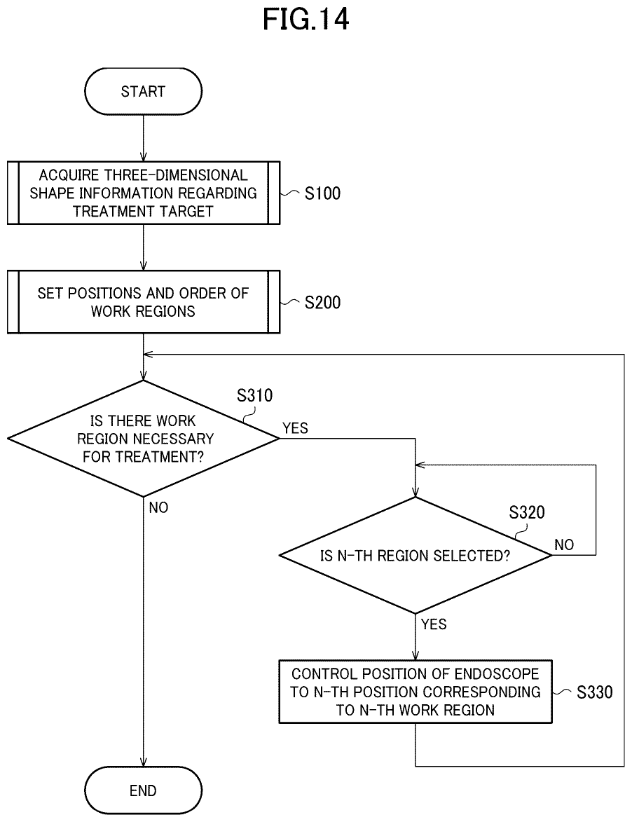

A processing example according to the method of the present embodiment is described with reference to a flowchart in FIG. 14. Processing described in FIG. 14 is applicable to both the first manipulation (step S10) and the second manipulation (step S20) in FIG. 13. However, processing details of step S200 in a case where the processing in FIG. 14 is applied to the first manipulation (step S10) and processing details of step S200 in a case where the processing in FIG. 14 is applied to the second manipulation (step S20) are partially different from each other. First, a brief description will be given below of the case where the processing in FIG. 14 is applied to the first manipulation (step S10), and thereafter a description will be given of the case where the processing in FIG. 14 is applied to the second manipulation (step S20). Additionally, regarding the case where the processing in FIG. 14 is applied to the second manipulation (step S20), a description will be given mainly of a point that is different from the case where the processing in FIG. 14 is applied to the first manipulation (step S10).

The processor 100 acquires three-dimensional shape information regarding the treatment target (step S100). A more detailed processing example of step S100 will be described later. Subsequently, the processor 100 sets the positions and order of work regions (step S200). Although details will be described later, processing in step S200 sets N work regions and the order of the N work regions.

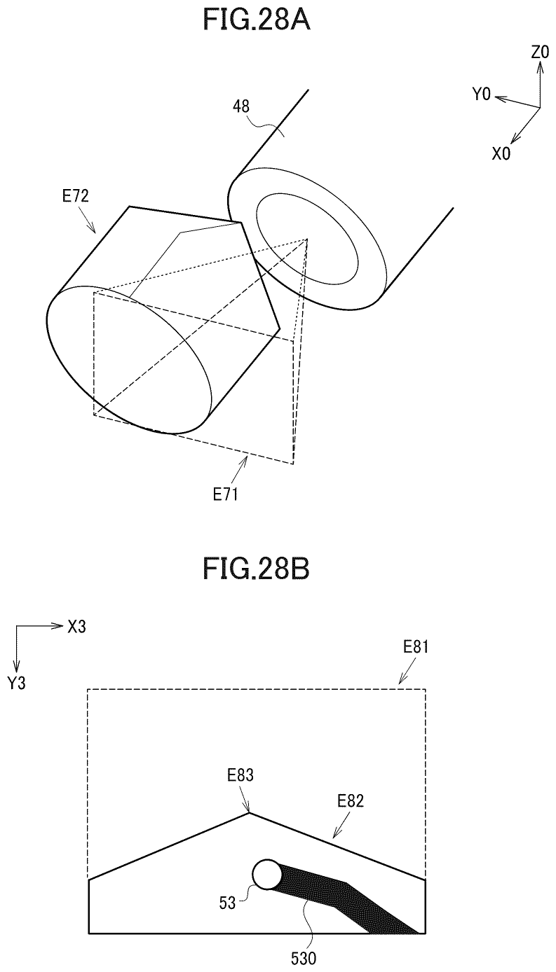

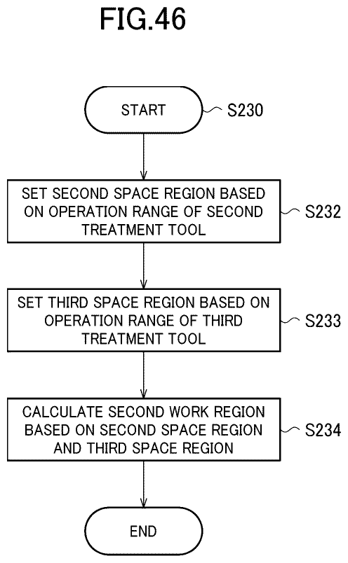

The work region in the present embodiment is a region associated with an operable range of the medical manipulator 500. The operable range is a range that satisfies both a range that is a predetermined range of the endoscope image and in which the treatment tool 50 associated with the medical manipulator 500 is visually recognizable, and a movable range of the medical manipulator 500. The predetermined range is a certain range that is appropriate for disposing the treatment tool 50 associated with the medical manipulator 500. In other words, the predetermined range can also be said as a range that is thought to be convenient for the user to visually recognize the treatment tool 50 at the time of performing the manipulation. A specific range of the predetermined range will be described later with reference to FIG. 28 and the like. In the following description, a work region in a case where the method of the present embodiment is applied to the first manipulation (step S10) is specifically referred to as a “first work region”. That is, the “first work region” is a movable range of the third medical manipulator 530 in a case where the first manipulation (step S10) is performed, and is a range in which the user can visually recognize the third treatment tool 53 in the endoscope image to such an extent as to allow the user to perform the first manipulation (step S10). Similarly, a work region in a case where the method of the present embodiment is applied to the second manipulation (step S20) is specifically referred to as a “second work region”, and details will be described later with reference to FIG. 46. Note that, in a case where a work region corresponding to both the “first work region” and the “second work region”, it is collectively referred to as a “work region”.

The work region in the present embodiment is position information regarding the movable range of the medical manipulator 500 and is set in association with the position information regarding the endoscope 40. That is, it is possible to set, theoretically, the number of work regions corresponding to the number of pieces of information regarding positions that can be taken by the endoscope 40. Although details will be described later, in the present embodiment, only work regions whose number is appropriate for performing the manipulation are selected, and serial numbers are allocated to the selected work regions. A work region that is selected as a first target of manipulation by the user is hereinafter referred to as a first work region. A work region that is selected as an N-th target of manipulation is more generally referred to as an N-th work region. Additionally, the N-th work region in the case where the method of the present embodiment is applied to the first manipulation (step S10) is hereinafter specifically referred to as a “first N-th work region”. The N-th work region in the case where the method of the present embodiment is applied to the second manipulation (step S20) is hereinafter specifically referred to as a “second N-th work region”. Note that, in a case where a work region corresponds to both the “first N-th work region” and the “second N-th work region”, it is collectively simply referred to as the “N-th work region”.

Thereafter, the processor 100 performs processing of determining whether there is a work region that is necessary for treatment (step S310). In a case of determining that there is the work region that is necessary for the treatment (YES in step S310), the processor 100 performs processing of determining whether the N-th work region is selected (step S320). In a case of determining that the N-th work region is selected (YES in step S320), the processor 100 then controls the position of the endoscope 40 to an N-th position corresponding to the N-th work region (step S330). In contrast, in a case of determining that the N-th work region is not selected (NO in step S320), the processor 100 performs step S320 again. That is, the processor 100 automatically controls the endoscope driving device 24 to move the endoscope 40 to positions of the respective work regions to which the serial numbers are added.

As described above, the position information regarding each work region is associated with the position of the endoscope 40. More specifically, a piece of position information regarding each work region and a piece of position information regarding the endoscope 40 are associated with each other and stored in the memory. A position indicated by the position information regarding the endoscope 40 associated with the position information regarding the first work region is hereinafter referred to as a “first position”. Similarly, a position indicated by the position information regarding the endoscope 40 associated with the position information regarding the N-th work region is referred to as an N-th position. Additionally, the N-th position in the case where the method of the present embodiment is applied to the first manipulation (step S10) is hereinafter specifically referred to as a “first N-th work position”. The N-th position in the case where the method of the present embodiment is applied to the second manipulation (step S20) is hereinafter referred to as a “second N-th position”. Note that, in a case where a position corresponds to both the “first N-th position” and the “second N-th position” it is collectively simply referred to as the “N-th position”.

For example, after the first to N-th work regions are set in step S200, the user designates the first work regions by performing an operation of the operation section of the console 60 or another operation to perform manipulation. Step S200 will be described later. At this time, the processor 100 controls the driving device 20 based on the stored position information regarding the first position and automatically moves the endoscope 40 to the first position. In other words, after designating the first work region to move the endoscope 40 to the first position, the user is only required to operate the first handle 641 while stepping on the first foot pedal 631 in order to handle the third treatment tool 53, which eliminates the need for operating another foot pedal 630 and another handle 640.

Thereafter, the user completes the operation of the manipulation, and thereafter operates, for example, the operating section, whereby the processor 100 moves the endoscope 40 from a first position to a second position. The operation section is not illustrated. Also in this case, the user need not operate the first handle 641 while stepping on the second foot pedal 632.

Note that the flowchart in FIG. 14 is applied to both the first manipulation (step S10) and the second manipulation (step S20) in FIG. 13. Thus, in a case where the processing according to FIG. 13 is performed, the processing according to the flowchart in FIG. 14 is executed twice. Hence, when performing step S330 for the first manipulation (step S10), the processor 100 performs drive control of the endoscope 40 to move to a first first position corresponding to a first first work region. Thereafter, when performing step S330 for the second manipulation (step S10), the processor 100 performs drive control of the endoscope 40 to move to a second first position corresponding to a second first work region.

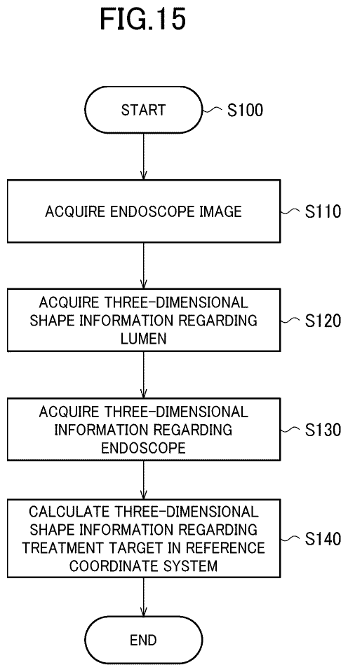

FIG. 15 is a flowchart describing a detailed processing example of processing in step S100 in FIG. 14. The processor 100 acquires an endoscope image (step S110). For example, the imager 42 captures an image of an inner wall of a lumen including a treatment target. The processor 100 then acquires information regarding the endoscope image from the imager 42. Although details will be described later, step S100 includes processing of recognizing a treatment target region from the captured endoscope image. Recognizing the treatment target region is, in other words, to recognize a boundary between the treatment target region and a region other than the treatment target. A boundary line associated with the boundary of the treatment target region, which is recognized by the processor 100 in step S110, is hereinafter referred to as a “boundary line of the treatment target” or simply referred to as a “boundary line”. The boundary line of the treatment target has meaning as an incision line serving as a target of incision with the third treatment tool 53.

Note that, in the following description, the imager 42 is exemplified by an imager that captures a two-dimensional image, but may be a three-dimensional camera such as a stereo camera. In this case, although a detailed description is omitted, part of processing described later can be omitted.

Thereafter, the processor 100 acquires three-dimensional shape information regarding the lumen (step S120). The three-dimensional shape information regarding the lumen includes three-dimensional shape information regarding the treatment target region. Step S120 can be implemented with use of a first sensor 410 or the like, and details will be described later. The first sensor 410 is not illustrated in FIG. 1.

Thereafter, the processor 100 acquires three-dimensional information regarding the endoscope 40 (step S130). The three-dimensional shape information regarding the endoscope 40 is three-dimensional shape information regarding a portion that is inserted into the inside of the body out of the endoscope 40, more specifically, three-dimensional shape information regarding the insertion portion of the endoscope 40 and the endoscope distal end. Step S130 can be implemented with use of a second sensor 420 or the like. The second sensor 420 is not illustrated in FIG. 1. As a result, it is possible for the processor 100 to acquire three-dimensional data that identifies the position and orientation of the endoscope distal end.

Thereafter, the processor 100 calculates three-dimensional shape information regarding the treatment target in a reference coordinate system (step S140). For example, the processor 100 uses the three-dimensional information regarding the lumen, which is acquired in step S120, and a parameter of the imager 42 to transform two-dimensional coordinates of the treatment target in the endoscope image into the three-dimensional information, and details of a method will be described later. Note that, although a detailed description regarding the reference coordinate system will be described later, values of the pieces of position information regarding the data acquired in steps S110, S120, and S130 are based on different measurement references, and thus processing of integrating the measurement references and transforming the values into values based on the integrated references is included in step S140. Then, based on the transformed three-dimensional shape information regarding the treatment target, set is three-dimensional shape information that identifies a portion to be incised by the user with use of the third treatment tool 53 in the first determination (step S10).

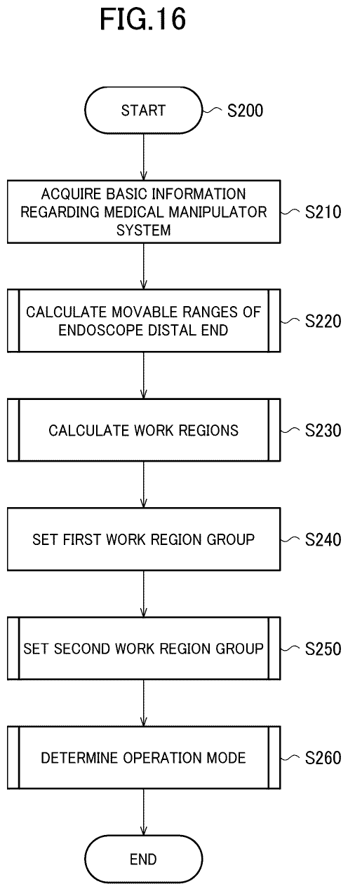

FIG. 16 is a flowchart describing a detailed processing example of processing in step S200 in FIG. 14. The processor 100 acquires basic information regarding the medical manipulator system 5 (step S210). Thereafter, the processor 100 calculates movable ranges of the endoscope distal end (step S220), and calculates work regions (step S230). Thereafter, the processor 100 sets a first work region group (step S240). For example, the processor performs processing of adding the work regions calculated in step S230 to the respective movable ranges calculated in step S220.

In the present embodiment, a set of a plurality of work regions is referred to as a work region group. A set of the plurality of work regions added to the respective movable ranges of the endoscope distal end in step S240 is distinctively referred to as a “first work region group”. Additionally, a set of a plurality of work regions with a possibility for being appropriate for manipulation is distinctively referred to as a “second work region group”. Furthermore, a set of a plurality of work regions for performing the manipulation is distinctively referred to as a “third work region group”. Since there is a case where an enormous number of work regions are included in the first work region group in a phase in which step S240 is performed, the number of work regions necessary for the manipulation is determined on a step-by-step basis. Although details will be described later, for example, the processor 100 performs processing in step S250 to select a plurality of work regions with a possibility of being appropriate for performing the manipulation from the first work region group and sets the second work region group. Additionally, the processor 100 selects a plurality of work regions for actually performing the manipulation from the second work region group, and sets the third work region group. That is, the first to N-th work regions are included in the third work region group.

Thereafter, the processor 100 determines an operation mode (step S260). Details of respective methods of performing steps S210, S220, S230, S240, S250, and S260 will be described later.

For the reasons described above, the medical manipulator system 5 in the present embodiment includes the endoscope 40 including the imager 42 that captures an endoscope image, the medical manipulator 500 that protrudes from the distal end surface of the endoscope 40, the driving device 20 that controls the endoscope 40 and the medical manipulator 500, and the processor 100. The processor 100 acquires the endoscope image in which the treatment target is seen from the imager 42, recognizes the treatment target region from the endoscope image, acquires the three-dimensional shape information regarding the lumen, and acquires the three-dimensional shape information regarding the endoscope 40. Additionally, the processor 100 calculates information regarding a work region that is an operable range of the medical manipulator 500 with respect to each of the plurality of positions of the endoscope 40, and thereby acquires information regarding a plurality of work regions. Furthermore, the processor 100 performs drive control of the endoscope 40 to move to the first position corresponding to the first work region selected from among the plurality of work regions based on the treatment target region, the three-dimensional shape information regarding the lumen, and the three-dimensional shape information regarding the endoscope 40.

In this manner, the medical manipulator system 5 in the present embodiment includes the imager 42, the medical manipulator 500, the driving device 20, and the processor 100, and is thereby applicable to the endoscope 40 for medical purposes. Additionally, the processor 100 calculates the information regarding the work region that is the operable range of the medical manipulator 500, which allows the user to grasp whether the treatment tool 50 associated with the medical manipulator 500 reaches the treatment target or other matters. Furthermore, the processor 100 performs drive control of the endoscope 40 to move to the first position corresponding to the selected first work region, which eliminates the user's burden to operate the endoscope 40.

The user performs incision so as to surround the lesion in peripheral incision, ESD, and the like among manipulation using the endoscope 40. Thus, the user has to perform the incision while changing the position of the endoscope distal end multiple times, which is a heavy burden on the user. The method disclosed in International Publication No. 2017/048194 does not give consideration to driving of the endoscope 40 to move to an optimum position each time depending on the progress of the manipulation. In this regard, by applying the method of the present embodiment, the endoscope 40 is automatically subjected to drive control to move to the N-th position corresponding to the preliminarily selected N-th work region, which makes it possible to shorten time necessary for adjustment of the position of the endoscope 40 and also reduce a burden on the user to perform the manipulation. This allows the user to concentrate on performing incision on the treatment target in the treatment.

Alternatively, the method of the present embodiment may be implemented by the processor 100. That is, the processor 100 of the present embodiment controls the endoscope 40 including the imager 42 that captures an endoscope image, the medical manipulator 500 that protrudes from the distal end surface of the endoscope 40, and the driving device 20 that controls the endoscope 40 and the medical manipulator 500. Additionally, the processor 100 acquires the endoscope image in which the treatment target is seen from the imager 42, recognizes the treatment target region from the endoscope image, acquires the three-dimensional shape information regarding the lumen, and acquires the three-dimensional shape information regarding the endoscope 40. Additionally, the processor 100 calculates the information regarding the work region that is the operable range of the medical manipulator 500 with respect to each of the plurality of positions in the movable range of the endoscope 40, and thereby acquires the information regarding the plurality of work regions. Furthermore, the processor 100 further performs drive control of the endoscope 40 to move to the first position corresponding to the first work region selected from among the plurality of work regions based on the treatment target region, the three-dimensional shape information regarding the lumen, and the three-dimensional shape information regarding the endoscope 40. With this configuration, it is possible to obtain an effect that is similar to the above-mentioned effect.

Alternatively, the method of the present embodiment may be implemented by a control method. That is, the control method of the present embodiment controls the endoscope 40 including the imager 42 that captures an endoscope image, the medical manipulator 500 that protrudes from the distal end surface of the endoscope 40, and the driving device 20 that controls the endoscope 40 and the medical manipulator 500. Additionally, the control method of the present embodiment includes acquiring the endoscope image in which the treatment target is seen from the imager 42, recognizing the treatment target region from the endoscope image, acquiring the three-dimensional shape information regarding the lumen, and acquiring the three-dimensional shape information regarding the endoscope 40. Additionally, the control method of the present embodiment includes calculating information regarding the work region that is the operable range of the medical manipulator 500 with respect to each of the plurality of positions in the movable range of the endoscope 40 to acquire the information regarding the plurality of work regions. Additionally, the control method of the present embodiment includes performing drive control of the endoscope 40 to move to the first position corresponding to the first work region selected from among the plurality of work regions based on the treatment target region, the three-dimensional shape information regarding the lumen, and the three-dimensional shape information regarding the endoscope 40. With this configuration, it is possible to obtain an effect that is similar to the above-mentioned effect.

Additionally, the processor 100 may acquire information regarding the work region that is a range virtually set based on a range that is within a predetermined range in the endoscope image and in which the treatment tool 50 associated with the medical manipulator 500 is seen and based on the movable range of the medical manipulator 500. This allows the processor 100 to perform drive control of the endoscope 40 to move to the position corresponding to the work region that is set in the endoscope image based on the range in which the treatment tool 50 is seen and the movable range of the medical manipulator 500.

Alternatively, the above-mentioned method may be implemented as a control method. That is, the control method of the present embodiment may include acquiring information regarding the work region that is the range virtually set based on the range that is within the predetermined range in the endoscope image and in which the treatment tool 50 associated with the medical manipulator 500 is seen and based on the movable range of the medical manipulator 500. With this configuration, it is possible to obtain an effect that is similar to the above-mentioned effect.

Additionally, the processor 100 may select the first work region and the second work region from among the plurality of work regions, move the endoscope 40 to the first position corresponding to the selected work region, and thereafter move the endoscope 40 to the second position corresponding to the selected second work region. This can further reduce the user's burden to perform the manipulation.

Alternatively, the above-mentioned method may be implemented as a control method. That is, the control method of the present embodiment may include selecting the first work region and the second work region from among the plurality of work regions and moving the endoscope 40 to the first position corresponding to the selected first work region, and thereafter moving the endoscope 40 to the second position corresponding to the selected second work region. With this configuration, it is possible to obtain an effect that is similar to the above-mentioned effect.

Additionally, the processor 100 may perform drive control of the endoscope 40 to move to the first first position corresponding to the first first work region selected from among the plurality of first work regions in the first manipulation (step S10). Additionally, the processor 100 may perform drive control of the endoscope 40 to move to the second first position corresponding to the second first work region selected from among the plurality of second work regions in the second manipulation (step S20). This makes it possible to construct the medical manipulator system 5 that sets different work regions depending on each manipulation in treatment with different types of continuous manipulation, and that performs drive control of the endoscope 40 to move to respective positions corresponding to the set work regions.

Alternatively, the above-mentioned method may be implemented as a control method. That is, the control method of the present embodiment may include performing drive control of the endoscope 40 to move to the first first position corresponding to the first first work region selected from among the plurality of first work regions in the first manipulation (step S10). Additionally, the control method of the present embodiment may include performing drive control of the endoscope 40 to move to the second first position corresponding to the second first work region selected from among the plurality of second work regions in the second manipulation (step S20). With this configuration, it is possible to obtain an effect that is similar to the above-mentioned effect.

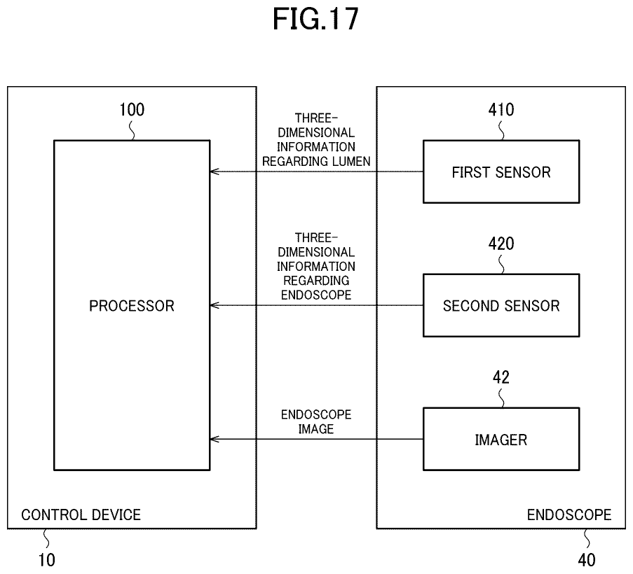

Details of the method regarding steps S110, S120, S130, and S140 in FIG. 15 will be described later. For example, the endoscope 40 includes, in addition to the above-mentioned imager 42, the first sensor 410 and the second sensor 420 as illustrated in FIG. 17, which makes it possible to implement the method associated with the processing example in FIG. 15. For example, the processor 100 acquires the endoscope image from the imager 42, which makes it possible to implement step S110. The processor 100 acquires the three-dimensional shape information regarding the lumen from the first sensor 410, which makes it possible to implement processing associated with step S120. The processor 100 acquires the three-dimensional shape information regarding the endoscope 40 from the second sensor 420, which makes it possible to implement processing associated with step S130.

In step S110, for example, the processor 100 performs processing of segmenting the treatment target region based on the acquired endoscope image. Note that, although illustration is omitted, step S110 may be performed with use of a trained model that has been subjected to machine learning. The trained model has been subjected to machine learning so that, for example, when the endoscope image in which the local injection (step S4) is performed in the periphery of the lesion is input to the trained model, the trained model performs segmentation or the like on the endoscope image to add a region that is appropriate for the first manipulation (step S10) or the second manipulation (step S20) and outputs the endoscope image. It is possible to use a convolutional neural network (CNN) or the like used in an image recognition field as the trained model associated with step S110. Alternatively, the user may be able to use a function of the touch panel 620 in FIG. 8 or the like to set the treatment target region. For example, the user operates the touch panel 620 to form an incision in the first manipulation (step S10) while seeing the endoscope image displayed on the touch panel 620, and can thereby determine the boundary line of the treatment target. Alternatively, the treatment target segmented by the processor 100 may be displayed on the touch panel 620, and the user may be able to correct the displayed treatment target region as appropriate using the touch panel 620.

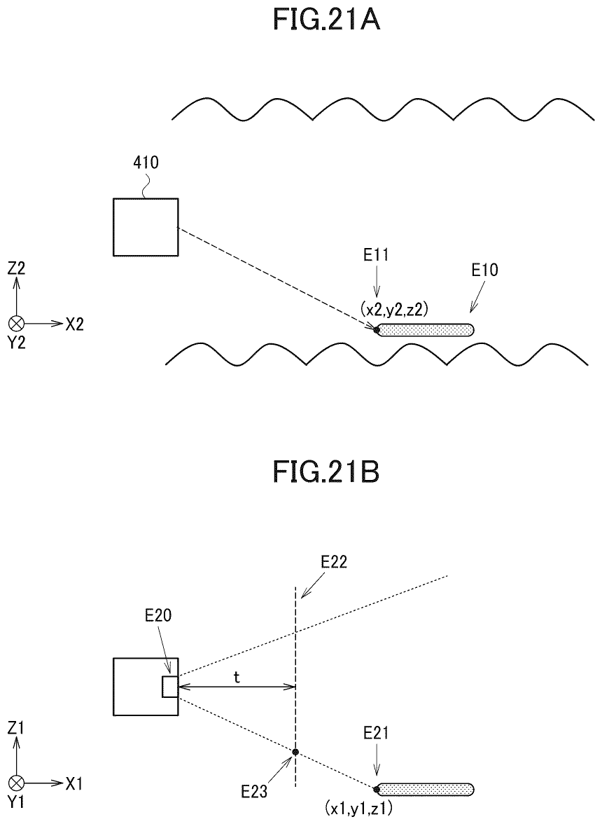

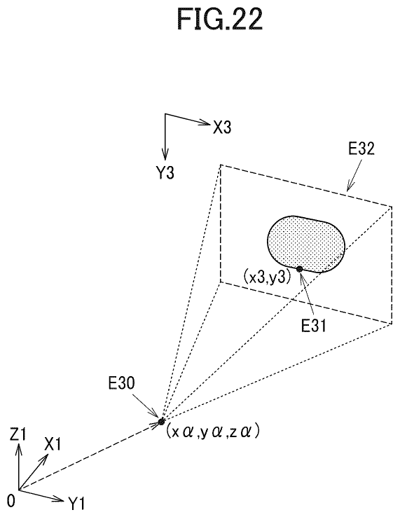

Note that, in step S110, the processor 100 performs processing of storing the position information regarding the treatment target region in the memory. Detailed examples will be described later with reference to FIGS. 21B and 22.

The method in step S120 is now described in detail. For example, the endoscope distal end includes a first sensor 410 as a distance-measurement sensor. The first sensor 410 is, more specifically, for example, a distance sensor that detects a distance image using a time-of-flight (TOF) method. The distance sensor measures time of flight of light to measure a distance. The first sensor 410 detects a distance from the endoscope distal end portion to the inner wall of the lumen on a pixel-by-pixel basis. It is possible to calculate position information regarding each point of the inner wall inside the body, that is, three-dimensional shape information regarding the lumen from the distance regarding each pixel detected by the first sensor 410 and position information and orientation information regarding the endoscope distal end.

Additionally, the first sensor 410 may be a distance sensor that detects a distance using a light detection and ranging (LiDAR) method. The first sensor 410 using the LiDAR method includes, for example, a laser light source, an optical sensor, and the like. The first sensor 410 emits laser light from the laser light source to the inner wall of the lumen, detects laser light, which is bounced off the inner wall of the lumen, with the optical sensor, and thereby measures time until light bounces off the inner wall of the lumen. This makes it possible to measure a distance to and a direction of the inner wall of the lumen. The processor 100 is capable of calculating the three-dimensional information regarding the lumen by, for example, acquisition or the like of information regarding the distance to the inner wall of the lumen as point group data.

Note that, since the above-mentioned TOF method and LiDAR method are well-known methods, illustration thereof is omitted. For the reasons described above, the medical manipulator system 5 in the present embodiment includes the first sensor 410, and the processor 100 acquires the three-dimensional shape information regarding the lumen from the first sensor 410. With this configuration, it is possible to construct the medical manipulator system 5 that acquires the three-dimensional shape information regarding the lumen with the first sensor 410.

Additionally, the first sensor 410 may be a distance sensor that detects a distance using the TOF method or the LiDAR method. With this configuration, it is possible to construct the medical manipulator system 5 that acquires the three-dimensional shape information regarding the lumen with the distance sensor using the TOF method or the LiDAR method.

Note that a method of calculating the three-dimensional information regarding the lumen is not limited to the above-mentioned method. For example, the processor 100 may acquire the three-dimensional shape information regarding the lumen from position information regarding pixels in a three-dimensional space. The position information regarding the pixels in the three-dimensional space is calculated based on triangulation from position information regarding pixels of feature points included in two captured images that have been simultaneously acquired with use of a stereo camera. Additionally, the processor 100 may calculate the position of each feature point using a photometric stereo image. In this case, the endoscope distal end is provided with a plurality of illumination windows. A plurality of beams of illumination light emitted from the plurality of illumination windows can be switched and selectively emitted by drive control of a plurality of light-emitting diodes for illumination provided in a light source device, which is not illustrated. A shadow portion in an image of the wall of the lumen changes in state by switching of illumination light. Thus, it is possible to calculate a distance to the shadow portion of the wall of the lumen based on an amount of the change. That is, it is possible to acquire the three-dimensional shape information regarding the lumen based on photometric stereo from an image of a shadow region in a captured image obtained by emission of a plurality of illumination sections that are selectively operated.

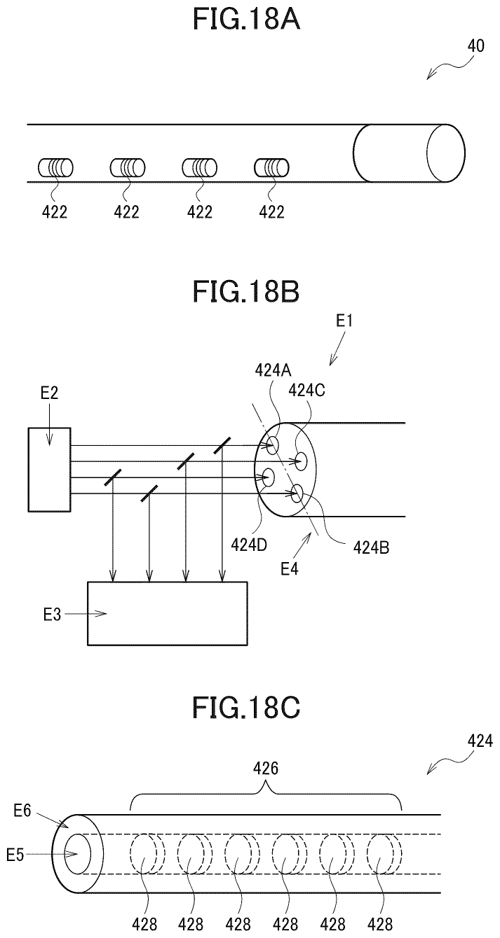

The method in step S130 is now described in detail. For example, the second sensor 420 is a bending shape observation sensor that employs a sensing method using a magnetic field. More specifically, for example, the second sensor 420 includes a plurality of source coils 422, a current generation device, a first insertion shape calculation device, and an antenna. The current generation device, the first insertion shape calculation device, and the antenna are not illustrated. For example, as illustrated in FIG. 18A, the plurality of source coils 422 is provided in the insertion portion of the endoscope 40 at predetermined intervals. The current generation device, which is not illustrated, causes each of the source coils 422 to sequentially output sine-wave current from a source coil 422 on the endoscope distal end side. Each source coil 422 generates a magnetic field from the current. The first insertion shape calculation device then detects the magnetic field generated from each source coil 72 via the antenna, which is not illustrated, and also acquires position information regarding each source coil 422 based on the intensity of the detected magnetic field. Additionally, the first insertion shape calculation device generates insertion shape information regarding the insertion portion of the endoscope 40 based on the acquired position information regarding each of the plurality of source coils 422, and transmits the generated insertion shape information to the processor 100. Note that, for example, the processor 100 may function as a second insertion shape calculation device, and it is possible to make various modifications.

Additionally, for example, the second sensor 420 may be a bending shape observation sensor that employs a sensing method using distortion. The second sensor 420 includes, for example, an optical fiber 424 and the second insertion shape calculation device, as described later. Although detailed illustration is omitted, the second insertion shape calculation device includes, for example, a predetermined light source device, a reflection light receiver, a spectroscope, a spectral analysis device, a curvature calculation device, and a bending shape calculation device.

The second sensor 420 includes a plurality of optical fibers 424. For example, as conceptually indicated by E1 in FIG. 18B, the endoscope insertion portion includes optical fibers 424A, 424B, 424C, and 424D. Note that in the following description, the optical fibers 424A, 424B, 424C, and 424D may be collectively and simply referred to as the optical fiber 424.

As illustrated in FIG. 18C, the optical fiber 424 is configured to have a core portion that is indicated by E5 and that is inserted through a clad portion indicated by E6. In the optical fiber 424 included in the second sensor 420, the core portion includes a fiber bragg grating (FBG) 426 (hereinafter also referred to as the FBG 426). The FBG 426 includes a bragg grating portion 428 that periodically changes in refractive index. With this configuration, when light with a specific wavelength is incident, it is possible to obtain reflection diffraction light with the bragg grating portion 428. Note that the specific wavelength is also called a bragg wavelength. In a state where the endoscope insertion portion is in a straight state, a value of the specific wavelength and a value of the wavelength of reflection diffraction light are equal. In contrast, for example, when the endoscope insertion portion bends, distortion occurs in the bragg grating portion 428, and the value of the wavelength of reflection diffraction light becomes larger or smaller than the value of the specific wavelength depending on a degree of distortion. In this regard, the FBG 426 is configured to include the bragg grating portions 428 arranged at predetermined intervals to have mutually different refractive indexes, whereby it is possible to acquire information regarding a direction and degree of distortion at a position corresponding to each of the bragg grating portions 428.

For example, predetermined incident light from the light source device indicated by E2 in FIG. 18B is incident on the optical fiber 424, whereby reflection diffraction light is taken from the optical fiber 424 into the reflection light receiver indicated by E3. Assume that predetermined incident light includes light based on a specific wavelength corresponding to each bragg grating portion 428. The reflection diffraction light taken into the reflection light receiver is detected by the spectroscope, whereby a predetermined spectral distribution can be obtained. The spectral analysis device detects the peak of predetermined reflection diffraction light based on the obtained predetermined spectral distribution, and obtains a wavelength at the detected peak. Curvature data is calculated based on a value of the wavelength at the detected peak and a value of the specific wavelength. The bending shape calculation device calculates bending shape data indicating the endoscope insertion portion based on the acquired curvature data and transmits the bending shape data to the processor 100. Note that the processor 100 may function as part of the second insertion shape calculation device.

Additionally, for example, optical fibers 424A and 424B are preferably disposed to face each other along a direction of a broken line indicated by E4 in FIG. 18B. With this configuration, for example, when the endoscope insertion portion bends, it is possible to obtain bending shape data in a case where the optical fiber 424A is located on one of the inner periphery side or outer periphery side of a bent portion and the optical fiber 424B is located on the other side. Consequently, it is possible to acquire the three-dimensional shape information regarding the endoscope insertion portion with higher accuracy. Similarly, it is preferable that optical fibers 424C and 424D be disposed to face each other in a direction that is perpendicular to a broken line indicated by E4 in FIG. 18B.

For the reasons described above, the medical manipulator system 5 in the present embodiment includes the second sensor 420, and the processor 100 acquires the three-dimensional shape information regarding the endoscope 40 from the second sensor 420. With this configuration, it is possible to construct the medical manipulator system 5 that acquires the three-dimensional shape information regarding the endoscope 40 with the second sensor 420.

Additionally, the second sensor 420 may be a bending shape observation sensor that employs a sensing method using a magnetic field or a bending shape observation sensor that employs a sensing method using distortion. With this configuration, it is possible to construct the medical manipulator system 5 that acquires the three-dimensional shape information regarding the endoscope 40 with the bending shape observation sensor that employs the sensing method using a magnetic field or distortion. Note that the second sensor 420 in the present embodiment may be, for example, a bending sensor that observes the shape that employs a sensing method using ultrasonic waves, or a bending sensor that observes the shape that employs a sensing method using an X-ray absorption material.

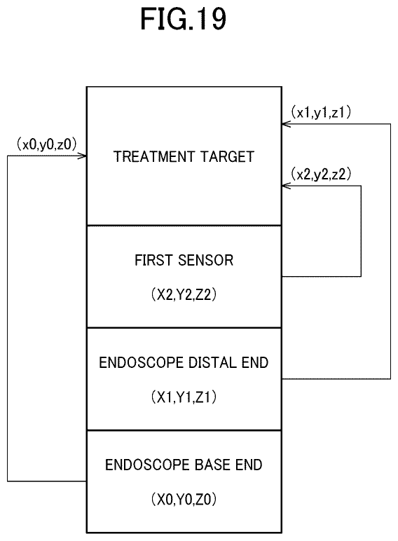

Subsequently, the calculation method associated with step S140 is conceptually described with reference to FIGS. 19 to 22. The position information is information that varies in measured value depending on a measurement reference. Specifically, for example, the position information regarding the treatment target is acquired as coordinate information regarding the endoscope image with the imager 42 included in the endoscope distal end in the above-mentioned step S110. Additionally, the three-dimensional shape information regarding the lumen is acquired with the first sensor 410 in step S120. With this processing, the three-dimensional shape information regarding the treatment target that exists in the inner wall of the lumen is acquired. In this case, a value of the position information regarding the treatment target and acquired in step S110, and a value of the position information regarding the treatment target and acquired in step S120 are different due to the above-mentioned reasons.

Thus, in the present embodiment, a coordinate system that serves as a reference for a measured value to be used for calculation associated with step S140 is referred to as a “reference coordinate system”. Three-dimensional coordinate axes in a left-handed system using a base end of the endoscope 40 as a reference, that is, three-dimensional coordinate axes in the left-handed system of the reference coordinate system are hereinafter referred to as an X0 axis, a Y0 axis, and a Z0 axis. Three-dimensional coordinate axes in the left-handed system using the endoscope distal end as a reference are hereinafter referred to as an X1 axis, a Y1 axis, and a Z1 axis. Additionally, three-dimensional coordinate axes in the left-handed system using the first sensor 410 as a reference are hereinafter referred to as an X2 axis, a Y2 axis, and a Z2 axis. In this case, as illustrated in FIG. 19, it is possible to express positional coordinates of the treatment target using the base end of the endoscope 40 as a reference, that is, positional coordinates of the treatment target in the reference coordinate system as (x0, y0, z0). Similarly, it is possible to express positional coordinates of the treatment target region in a case of using the endoscope distal end as a reference as (x1, y1, z1), and it is possible to express positional coordinates of the treatment target region in a case of using the first sensor 410 as a reference as (x2, y2, z2).

For example, a relationship expressed by the following Expression 1 using a transformation matrix M (12) holds between the position information (x1, y1, z1) and the position information (x2, y2, z2).

$$\begin{matrix}[ Expression 1 ] & \\ ( \begin{matrix}x 1 \\ y 1 \\ z 1\end{matrix} ) = M ( 12 ) ( \begin{matrix}x 2 \\ y 2 \\ z 2\end{matrix} ) & ( 1 )\end{matrix}$$

Similarly, a relationship expressed by the following Expression 2 using a transformation matrix M (01) holds between the position information (x0, y0, z0) and the position information (x1, y1, z1).

$$\begin{matrix}[ Expression 2 ] & \\ ( \begin{matrix}x 0 \\ y 0 \\ z 0\end{matrix} ) = M ( 01 ) ( \begin{matrix}x 1 \\ y 1 \\ z 1\end{matrix} ) & ( 2 )\end{matrix}$$

Similarly, a relationship expressed by the following Expression 3 using Expressions 1 and 2 holds between the position information (x0, y0, z0) and the position information (x2, y2, z2).

$$\begin{matrix}[ Expression 3 ] & \\ ( \begin{matrix}x 0 \\ y 0 \\ z 0\end{matrix} ) = M ( 01 ) M ( 12 ) ( \begin{matrix}x 2 \\ y 2 \\ z 2\end{matrix} ) & ( 3 )\end{matrix}$$

For example, in a case where the inner wall of the lumen is the inner wall of the large intestine, a relationship between the X0 axis, the Y0 axis, and Z0 axis and the X1 axis, the Y1 axis, and Z1 axis is, for example, as illustrated in FIG. 20, and the orientations of the X1 axis, the Y1 axis, and Z1 axis constantly change depending on onward movement of the endoscope insertion portion, a situation of the treatment, and the like. In the present embodiment, the three-dimensional shape information regarding the endoscope 40 is periodically acquired in step S130. This makes it possible to grasp the relationship between the X0 axis, the Y0 axis, and Z0 axis and the X1 axis, the Y1 axis, and Z1 axis, whereby it is possible to acquire the position information regarding the treatment target as values in the reference coordinate system. That is, it is possible to obtain the transformation matrix (01) in Expression 2 in step S130.

Subsequently, a description is given of a method of expressing the position of the treatment target whose image is captured in the endoscope image using the reference coordinate system. For example, as conceptually illustrated in FIG. 21A, in addition to the three-dimensional shape information regarding the lumen, position information regarding a treatment target indicated by E10 is acquired with the first sensor 410. Since these pieces of information are information acquired using the first sensor 410 as a reference, it is possible to express, for example, positional coordinates of a position indicated by Ell and associated with a position included in a region of the treatment target indicated by E10 as (x2, y2, z2).

Meanwhile, the image of the treatment target is also captured by the imager 42 included in the vicinity of the endoscope distal end. In this case, it is possible to express the position information regarding the treatment target using the endoscope distal end as a reference. Thus, for example, as illustrated in FIG. 21B, in a case where a coordinate system including the X1 axis, the Y1 axis, and the Z1 axis is set, it is possible to express position information regarding a position indicated by E21 as (x1, y1, z1). Since the position indicated by E21 in FIG. 21B is identical to the position indicated by E11 in FIG. 21A but is different in measurement reference, values of (x2, y2, z2) in FIG. 21A are different from respective values of (x1, y1, z1) in FIG. 21B.

Additionally, in a case where the image of the treatment target is captured with the imager 42, an image is formed at a position that is away by a distance t in a +X1 direction from a lens position indicated by E20 in FIG. 21B. Note that t is basic information regarding the imager 42, and a known value. The basic information regarding the imager 42 will be described later. That is, it is possible to regard that there is a plane of the treatment target at the position that is away by the distance t in the +X1 direction from the lens position. In a case where the treatment target is seen at a position indicated by E23 in a virtual image indicated by E22, the treatment target is located at the position indicated by E21 on an extended line of a line that connects the lens position indicated by E20 and the position indicated by E23 (line at an angle of view of the imager 42). That is, in a case where the lens position indicated by E20 serves as a reference, it is possible to obtain the position information regarding the treatment target indicated by E21 by performing scale transformation of the position information associated with the position indicated by E23.

Additionally, a screen indicated by E22 in FIG. 21B corresponds to a screen indicated by E32 in FIG. 22 when conceptually illustrated in a perspective view. Here, a position indicated by E30 in FIG. 22 corresponds to a lens position indicated by E20 in FIG. 21B, and a position indicated by E31 in FIG. 22 corresponds to a position indicated by E23 in FIG. 21B. Additionally, an X3 axis and a Y3 axis in FIG. 22 are coordinate axes of the screen indicated by E32. That is, the position information regarding the treatment target and stored in the above-mentioned step S110 includes a value based on the X3 axis and a value based on the Y3 axis in FIG. 22. For example, the position information regarding the position indicated by E31 in FIG. 22 can be represented as position information (x3, y3) on the endoscope image.

Additionally, in FIG. 22, the lens position information indicated by E30 is expressed as coordinates (xα, yα, zα) using the coordinate system including the X1 axis, the Y1 axis, and the Z1 axis as a reference.

For the reasons described above, it is found that a relationship expressed by the following Expression 4 holds between (x1, y1, z1) in FIG. 21B and (x3, y3) in FIG. 22.

$$\begin{matrix}[ Expression 4 ] & \\ ( \begin{matrix}x 1 \\ y 1 \\ z 1\end{matrix} ) = ( \begin{matrix}x α \\ y α \\ z α\end{matrix} ) + s ( \begin{matrix}t \\ x 3 \\ - y 3\end{matrix} ) & ( 4 )\end{matrix}$$

That is, when the position information (x1, y1 z1), the position information (xα, yα, zα), and a value of t are specifically found, it is possible to obtain a value of s associated with scale transformation.

Additionally, assuming that the right side of Expression 1 and the right side of Expression 4 are equal, the following Expression 5 holds.

$$\begin{matrix}[ Expression 5 ] & \\ M ( 12 ) ( \begin{matrix}x 2 \\ y 2 \\ z 2\end{matrix} ) = ( \begin{matrix}x α \\ y α \\ z α\end{matrix} ) + s ( \begin{matrix}t \\ x 3 \\ - y 3\end{matrix} ) & ( 5 )\end{matrix}$$

When values of the position information (x2, y2, z2) are specifically found from Expression 5, it is possible to obtain the transformation matrix M (12). As a result, from Expression 3, it is possible to transform the position information (x2, y2, z2) acquired with the first sensor 410 into the position information (x0, y, z0) in the reference coordinate system.

In this manner, it is possible to transform the position information acquired with the first sensor 410 and the position coordinates regarding the endoscope distal end into values using the reference coordinate system as a reference. In step S140, the processor 100 then transforms values of position information that identifies the treatment target into values based on the reference coordinate system, and stores a set of transformed values in the memory.

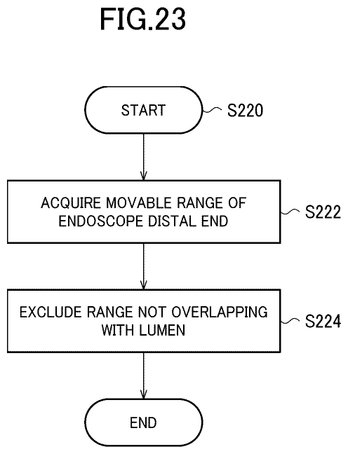

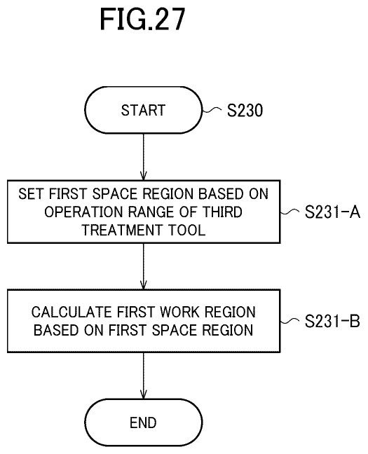

In the present embodiment, other than the treatment target, values of pieces of information acquired in steps S110, S120, and S130 are all transformed into values in the reference coordinate system. That is, calculation processing in FIG. 23, FIG. 27, and the like is performed by the processor 100 based on the values transformed into the values measured from the reference coordinate system.

Step S210 is now described in detail. Examples of the basic information regarding the medical manipulator system 5 include basic information regarding the endoscope 40, basic information regarding the imager 42, and basic information regarding the medical manipulator 500. The basic information regarding the endoscope 40 is information regarding a degree of freedom of the endoscope 40, a length, inner diameter, and outer diameter of the endoscope insertion portion, and the like. Examples of the basic information regarding the imager 42 include information regarding the position and angle of view of the imager 42. Examples of the basic information regarding the medical manipulator 500 include information regarding the degree of freedom, dimension, and operation range of the medical manipulator 500.

Step S210 can be implemented by various methods. For example, step S210 may be executed by the user's operation of the operation section or the like. For example, when the endoscope 40 or the like is connected to the driving device 20, it may be possible to automatically acquire information regarding the connected endoscope 40 or the like.

Step S220 is described in detail with reference to FIGS. 23 to 26. More specifically, step S220 is implemented as a processing example indicated by a flowchart in FIG. 23. The processor 100 acquires a movable range of the endoscope distal end (step S222). The movable range of the endoscope distal end mentioned herein is an overall range of positions and orientations to be taken by the endoscope distal end. Thereafter, the processor 100 excludes a region that does not overlap with the lumen from the range acquired in step S222 (step S224).

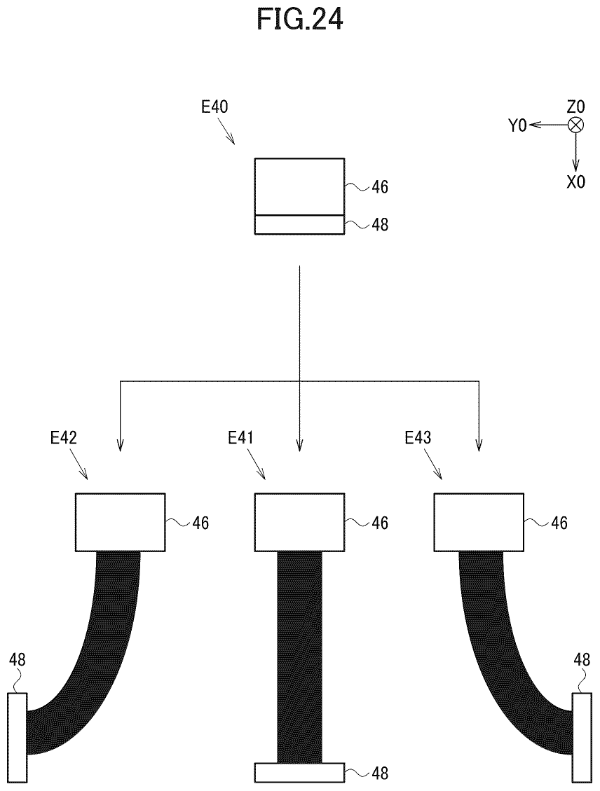

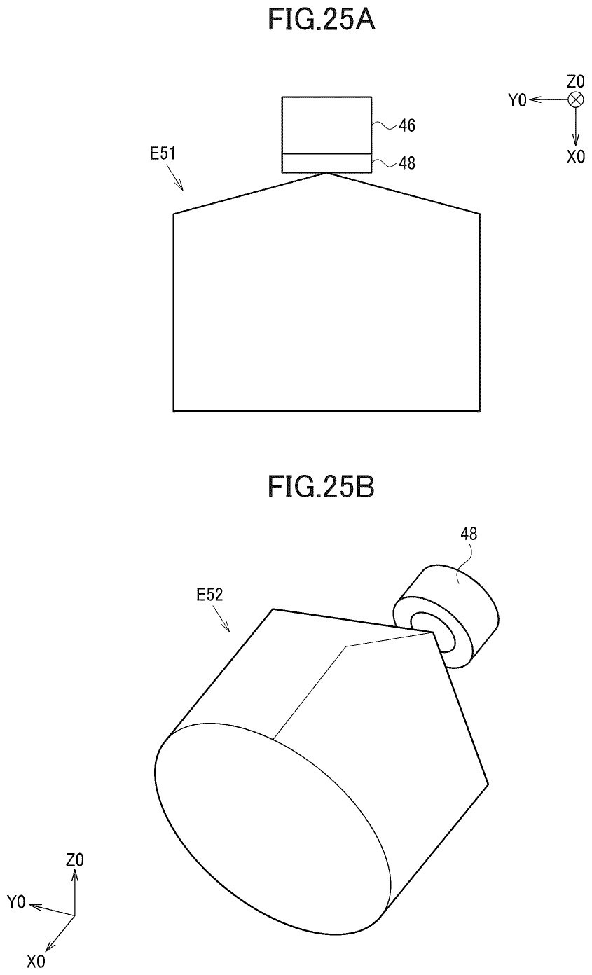

Step S222 is conceptually described with reference to FIGS. 24 and 25. Assume that the reference coordinate system is set as the X0 axis, the Y0 axis, and Z0 axis illustrated in FIG. 24 and subsequent drawings as appropriate so that a plane including a boundary line of the treatment target is parallel to an X0-Y0 plane for easier understanding of the gist of the present embodiment. For simplification of description, assume that a direction along the inner wall of the lumen is parallel to the X0 direction. Assume that the orientation of the endoscope 40 is adjusted so that the treatment target is seen on a lower side of the endoscope screen similarly to FIGS. 12, 21A, and 21B. Additionally, assume that the endoscope 40 moves forward in a direction parallel to the +X0 direction unless otherwise described.

The endoscope 40 illustrated in drawings to be used in the following description is represented by the endoscope 40 described above with reference to FIG. 5. That is, the endoscope 40 that can be independently displaced from the overtube 46 from a state where the overtube 46 and the cap 48 are integrated is used in the following description. Additionally, assume that the endoscope 40 to be used in the following description has a configuration in which the three treatment tools 50 (the first treatment tool 51, the second treatment tool 52, and the third treatment tool 53) can protrude via the cap 48 or the endoscope distal end. That is, assume that, in a case where the overtube 46 is fixed with a balloon or the like, the endoscope distal end protrudes with the distal end of the overtube 46 serving as a reference and can move forward or bend, and assume that, in a case where the endoscope distal end is fixed, the treatment tool 50 protrudes with the endoscope distal end servings as a reference and can move forward or bend. Additionally, in the drawings to be used in the following description, the endoscope distal end is represented by the cap 48 among components included in the endoscope distal end, and illustration of the other components is omitted as appropriate.

Note that, for example, in a case of the endoscope 40 in which the cap 48 is not movable independently of the overtube 46, the method of the present embodiment may be applied to control of the position of the endoscope 40 instead of control of the position of the overtube 46. This is because it is important to make the treatment tool 50 appropriately approach the treatment target and control a position of a portion including the medical manipulator 500. Hence, while an example of controlling the position of the endoscope 40 is described below, it is possible to think that the method of the present embodiment controls the position of the medical manipulator system 5 in a broader sense.

For example, as indicated by E40 in FIG. 24, assume a state where the overtube 46 and the cap 48 are integrated with each other. In this case, the user fixes the overtube 46 with the balloon or the like and operates a first part 651 of the first handle 641 along a direction indicated by D65 in FIG. 10 while stepping on the second foot pedal 632, which makes it possible to control the endoscope driving device 24 and perform a forward movement operation to move the endoscope 40 forward. This makes it possible to further separate the position of the endoscope distal end from the position of the distal end of the overtube 46 in the +X0 direction, as indicated by, for example, E41 in FIG. 24.

Additionally, the user operates the first part 651 of the first handle 641 along a direction indicated by D62 in FIG. 10 while stepping on the second foot pedal 632, which makes it possible to control the endoscope driving device 24 and perform bending motion to bend the endoscope bending portion in a direction along the Y0 direction. That is, the user can perform an operation that combines the above-mentioned forward movement operation and bending motion on the endoscope bending portion, which makes it possible to separate the position of the endoscope distal end from the position of the distal end of the overtube 46 as indicated by E42 or E43 in FIG. 24.

That is, in consideration of the forward movement operation in the +X0 direction and the bending motion in the direction along the Y0 direction, a reachable range of the endoscope distal end from the position of the distal end of the overtube 46 is like a range conceptually indicated by E51 in FIG. 25A. Additionally, the user operates the second part 652 of the first handle 641 along a direction indicated by D61 in FIG. 10 while stepping on the second foot pedal 632, which makes it possible to control the endoscope driving device 24 and perform bending motion to bend the endoscope bending portion in a direction along the Z0 direction. In this manner, in further consideration of the bending motion of the endoscope bending portion in the direction along the Z0 direction, a conceptual shape of the movable range of the endoscope distal end is like a range indicated by E52 in FIG. 25B.

The range indicated by E52 in FIG. 25B is not data obtained by measurement, but can be obtained from structure data of each portion that constitutes the endoscope 40. The structure data is included in the data acquired in step S210.

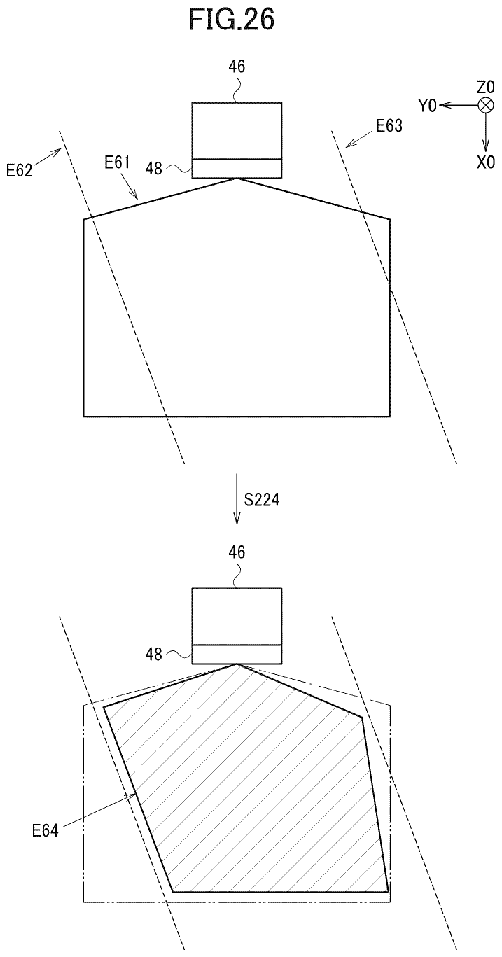

Step S224 is now described on a conceptual basis. For example, assume that a range indicated by E61 in FIG. 26 is the range calculated in step S222, and a region between a dotted line indicated by E62 in FIG. 26 and a dotted line indicated by E63 in FIG. 26 is a region of the lumen. It is impossible to assume that the endoscope distal end is driven to a region other than the region of the lumen. In step S224, the processor 100 then excludes a range that does not overlap with the lumen from the range calculated in step S222. Specifically, for example, the processor 100 performs processing of obtaining a set of pieces of position information in which a piece of three-dimensional shape information regarding the lumen acquired in step S120 and a piece of position information obtained by calculation in step S222 are matched with each other. As a result, a range indicated by E64 in FIG. 26 and a range other than the range indicated by E64 are obtained. The processor 100 then excludes the range other than the range indicated by E64 from the range indicated by E61 in FIG. 26 in step S224. With this processing, the range indicated by E64 in FIG. 26 is calculated as the movable range of the endoscope distal end.

Step S230 is described in more detail with reference to FIGS. 27 to 30. More specifically, step S230 is implemented as a processing example described in a flowchart in FIG. 27. The processor 100 sets a first space region based on an operation range of the third treatment tool 53 (step S231-A). Thereafter, the processor 100 calculates a first work region based on the first space region (step S231-B).

Step S231-A is now described on a conceptual basis. As described above, the third medical manipulator 530 that drives the third treatment tool 53 is configured to be capable of moving forward/backward and bending similarly to the endoscope 40. Hence, the processor 100 calculates the first space region as a mechanically operable range of the third treatment tool 53 based on the data acquired in step S210. A mechanism of forward/backward movement and bending of the third medical manipulator 530 is similar to a mechanism of forward/backward movement and bending of the endoscope 40, an outline of the first space region is like a range defined by a solid line indicated by E72 in FIG. 28A, similarly to a range indicated by E52 in FIG. 25. Note that the outline of a range in which imaging can be performed by the imager 42 is like a range defined by a dotted line indicated by E71 in FIG. 28A. Note that, as illustrated in FIG. 28A, the range indicated by E71 and the range indicated by E72 are illustrated on a conceptual basis, and do not specifically identify respective sizes of the ranges.

Step S231-B is now described on a conceptual basis. As described above, the processor 100 calculates, as the first work region, the range that satisfies both the range that is in the predetermined range of the endoscope image and in which the treatment tool 50 associated with the medical manipulator 500 can be visually recognized, and the first space region. For example, the user performs the first manipulation (step S10) while seeing the endoscope image, but is not necessarily able to perform the first manipulation (step S10) only by visually recognizing the third treatment tool 53 at a freely-selected position in the endoscope image. Thus, it is preferable that the third treatment tool 53 can be visually recognized in the predetermined range in the endoscope image.

Thus, in step S231-B, the processor 100 sets the predetermined range, which is a certain range that is appropriate for disposition of the treatment tool 50 associated with the medical manipulator 500, based on the endoscope image. The predetermined range can be determined as appropriate depending on how an image of the treatment target and an image of the inner wall are captured in the endoscope image. As described above, assuming that a plane including the boundary line of the treatment target is parallel to the X0-Y0 plane, for example, a rectangular region that is lower two-thirds of the endoscope image or a rectangular region that is lower half of the endoscope image may be the range that is appropriate for the treatment, and the ratio may be able to be freely determined by the user. Additionally, the predetermined range may not be the rectangular region, and may be, for example, an arc-shaped region. Alternatively, for example, in a case where the endoscope image having a rectangular shape is regarded as a clock face, a region between a first time and a second time with a centroid position of a frame of the endoscope image serves as the center may be the predetermined range. For example, in FIG. 28B, a rectangular region defined by a dotted line indicated by E81 is the endoscope image, and a region defined by a solid line indicated by E82 is an example of a region to be set as the predetermined range between a four o'clock direction and an eight o'clock direction with the centroid position of the frame of the endoscope image serving as the center. The centroid position is indicated by E83. Note that the four o'clock direction is a direction of four o'clock in a case where the endoscope image is regarded as the clock face of a 12-hour clock. Similarly, the eight o'clock direction is a direction of eight o'clock in a case where the endoscope image viewed from the endoscope distal end side is regarded as the clock face of the 12-hour clock. Note that FIG. 28B is merely an example, and does not limit the first time to four o'clock and the second time to eight o'clock. Then, in a case of setting the range defined by the solid line indicated by E82 in FIG. 28B as the predetermined range, the processor 100 calculates a range in which the range defined by the solid line indicated by E72 in FIG. 28A and the range defined by the solid line indicated by E82 in FIG. 28B overlap with each other to be the first work region. In this manner, in the medical manipulator system 5 in the present embodiment, the processor 100 superimposes the range that is in the certain range appropriate for disposition of the treatment tool 50 associated with the medical manipulator 500 in the endoscope image and in which the treatment tool 50 associated with the medical manipulator 500 is seen and the movable range of the medical manipulator 500 on each other, and thereby acquires information regarding the work region that is the virtually set range. This makes it possible to perform drive control of the endoscope 40 to move to the position corresponding to the work region that is set based on the range in which the treatment tool 50 is seen and the movable range of the third medical manipulator 530 so as to facilitate the user's manipulation in the endoscope image.

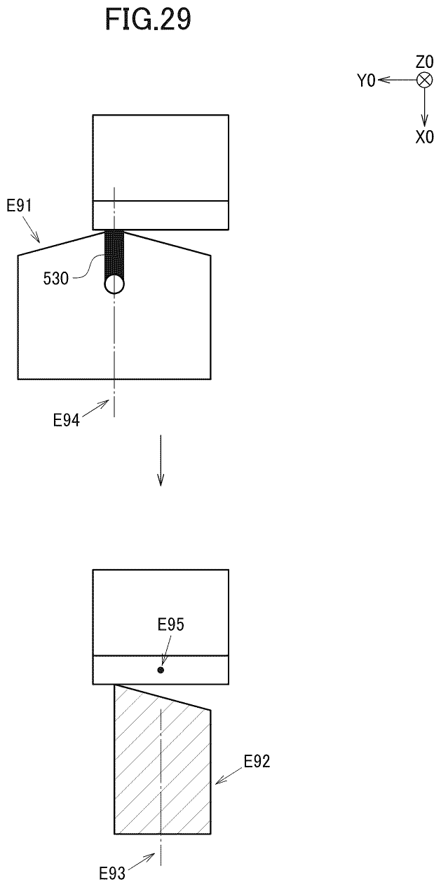

The first space region calculated in step S231-A is reduced in step S231-B. For example, in a case where the first space region is seen in the X0-Y0 plan view, the first space region calculated in step S231-A is assumed to be expressed by a region indicated by E91 in FIG. 29. Then, the region indicated by E91 is reduced to a region indicated by E92 in step S231-B, and the region indicated by E92 corresponds to the first work region.

Note that, for example, a direction indicated by E93 may be hereinafter referred to as a “direction that the first work region faces” for convenience. The direction indicated by E93 is a direction that is identical to a direction of forward/backward movement of the third medical manipulator 530 as indicated by E94. In other words, the direction that the first work region faces is identical to a direction that the endoscope distal end faces when the position of the endoscope 40 is controlled based on the first work region.

Additionally, each of the first space region indicated by E91 and the first work region indicated by E92 in FIG. 29 is illustrated to have such a shape as that the direction of the forward/backward movement of the third medical manipulator 530 is a longitudinal direction, but it is merely an example, and a specific size of the first work region is determined from a relationship with each component included in the endoscope 40.

Additionally, when performing step S231-B, the processor 100 may store, in the memory, the position information that identifies the first work region and the position information regarding the endoscope distal end at the time of execution of step S231-B in association with each other. In the following description, there is a case where a point indicating the position of the endoscope distal end is added to the cap 48 as indicated by E95 in FIG. 29 for explanatory convenience. However, where to locate the position of the endoscope distal end may be determined by the user as appropriate.

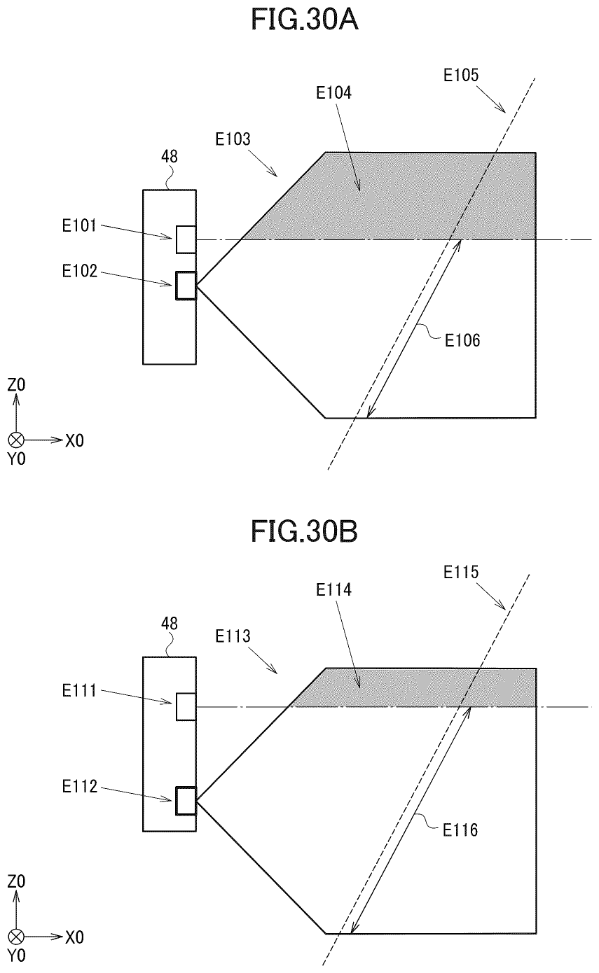

Processing in step S231-B when the first space region is viewed in the X0-Z0 plan view will be described with reference to FIG. 30 on a conceptual basis. For example, as illustrated in FIG. 30A, there is a case where the lens of the imager 42 is located at a position indicated by E101, the medical manipulator 500 protrudes from a position indicated by E102, and the predetermined range described above with reference to FIG. 28B is set at, for example, a rectangular region in the lower half of the endoscope image. In this case, a region indicated by E103 is calculated as the first space region. Additionally, a region indicated by E104 is not superimposed on the predetermined range, and thus is excluded from the first space region.

Additionally, for example, as illustrated in FIG. 30B, assume a case where the lens of the imager 42 is located at a position indicated by E111, the medical manipulator 500 protrudes from a position indicated by E112, and the predetermined range described above with reference to FIG. 28B is set at, for example, a rectangular region in the lower half of the endoscope image. In this case, a region indicated by E113 is calculated as the first space region. Additionally, a region indicated by E114 is not superimposed on the predetermined range, and thus is excluded from the first space region.

In this manner, a degree of reduction of the first space region in the Z0 direction changes in step S231-B depending on a relative positional relationship between the lens position of the imager 42 and the protrusion position of the medical manipulator 500. For example, if the position of the treatment target, a structure of the lumen at which the treatment target is located, and the like are found by an inspection result or the like before the treatment, it is possible for the user to preliminarily grasp information regarding how the treatment target is seen in the endoscope image and how much amount of reduction of the first space region in the Z0 direction is permitted. This allows the user to determine a relationship between the position of the endoscope 40 and the position of each medical manipulator 500 based on the grasped information before the endoscope 40 is inserted into the inside of the body. For example, as for the treatment tool 50 that allows the reduction of the first space region in the Z0 direction to some extent as illustrated in FIG. 30A, there is no problem even if the position of the endoscope 40 and the protrusion position of the medical manipulator 500 are relatively close to each other. Thus, the medical manipulator 500 is only required to be inserted through the treatment tool insertion opening 44. In contrast, as for the treatment tool 50 in which an amount of reduction of the first space region in the Z0 direction is desired to be small as illustrated in FIG. 30B, it is preferable that the position of the imager 42 and the protrusion position of the medical manipulator 500 be relatively far from each other. Thus, the medical manipulator 500 is only required to be inserted through a tube that forms a treatment tool channel outside the endoscope 40.

Additionally, as described above, the user may prepare the plurality of types of caps 48 that is different in relationship between the position of the endoscope exit hole and the position of each treatment tool exit hole and select an appropriate cap 48 from the plurality of types of caps 48. For example, in a case where the position of the imager 42 and the protrusion position of each medical manipulator 500 are desired to be relatively far from each other, the user is only required to select a cap 48 in which the position of the endoscope exit hole and the position of each treatment tool exit hole are at a relatively far distance therebetween.

Similarly, in a case where the medical manipulator system 5 that does not include the cap 48 is adopted, a plurality of types of overtubes 46 that is different in relationship between the position of the endoscope insertion portion channel and the position of each of the plurality of treatment tool channels may be prepared to allow the user to select an appropriate overtube 46 from among the plurality of types of overtubes 46. For example, in a case where the position of the imager 42 and the protrusion position of each medical manipulator 500 are desired to be relatively far from each other, the user is only required to select an overtube 46 in which the position of the endoscope insertion portion channel and the position of each treatment tool channel are at a relatively far distance therebetween.

Additionally, for example, after the insertion of the endoscope 40 into the inside of the body, driving the endoscope 40 to roll makes it possible to also adjust the relationship between the lens position of the imager 42 and the protrusion position of the medical manipulator 500.

Various factors are taken into consideration to determine how to adjust the relationship between the lens position of the imager 42 and the protrusion position of the medical manipulator 500. Other than the user's experience, the relationship between the protrusion position of the medical manipulator 500 and the position of the treatment target, and the like, examples of the various factors include the following factors.

For example, in a case where there is the inner wall indicated by E105 as illustrated in FIG. 30A, it is possible to cause the treatment tool 50 to reach a range indicated by E106. Similarly, in a case where there is the inner wall indicated by E115 as illustrated in FIG. 30B, it is possible to cause the treatment tool 50 to reach a range indicated by E116. The range indicated by E116 is larger than the range indicated by E106. Thus, in a case where the direction that the endoscope distal end faces and the direction along the inner wall are not parallel to each other, there is a case where it is convenient to adjust the relationship between the lens position of the imager 42 and the protrusion position of the medical manipulator 500.

Note that, while the description has been given of the case where the direction that the endoscope distal end faces and the direction along the inner wall are not parallel to each other in FIG. 30, it is preferable that the direction that the endoscope distal end faces and the direction along the inner wall be parallel to each other to perform the first manipulation in the present embodiment (step S10). However, the direction that the distal end of the overtube 46 faces and the direction along the inner wall are not necessarily required to be parallel to each other.

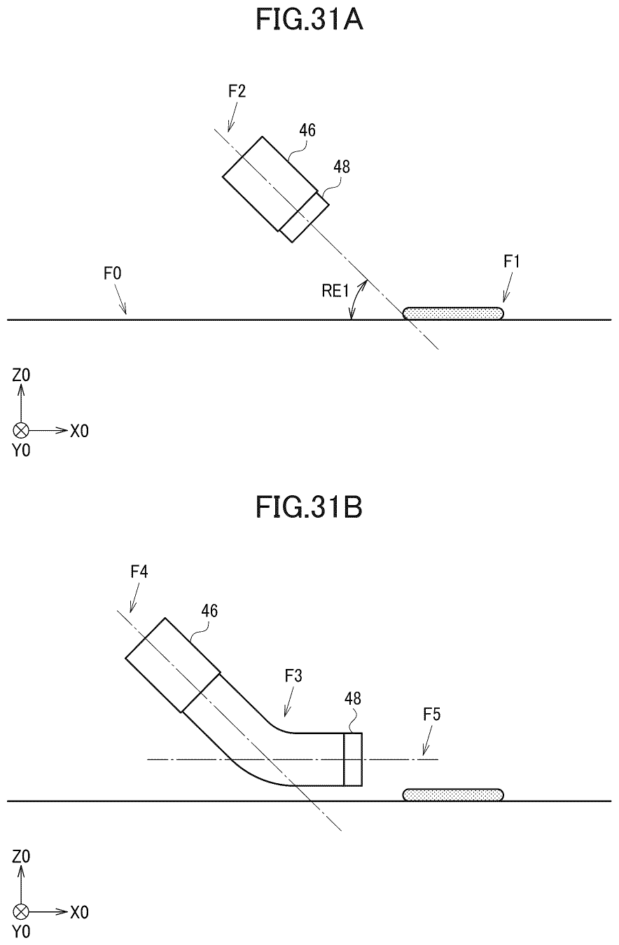

For example, as illustrated in FIG. 31A, assume that the overtube 46 is located so that an angle between the direction indicated by FO along the inner wall of the lumen and the direction that the distal end of the overtube 46 faces is an angle REI, and the overtube 46 is unable to move forward further due to factors such as the structure of the lumen. At this time, for example, in a case where the endoscope 40 having the cap 48 that is unable to be displaced independently of the overtube 46 is used, the third treatment tool 53 is caused to reach only a location associated with a line indicated by F2, and the forward/backward movement of the overtube 46 need be performed again. This is not convenient enough.

In this regard, the endoscope 40 in the present embodiment is capable of performing drive control of the endoscope insertion portion so as to allow the cap 48 to be displaced independently of the overtube 46. For this reason, as indicated by F3 in FIG. 31B, it is possible to bend the endoscope bending portion to make the direction that the endoscope distal end faces along the inner wall. In this case, a straight line indicated by F4 and a straight line indicated by F5 are different in orientation. The straight line indicated by F4 is a straight line that faces a direction that is identical to the direction that the distal end of the overtube 46 faces, and the straight line indicated by F5 is a straight line that faces a direction is identical to the direction that the endoscope distal end faces.

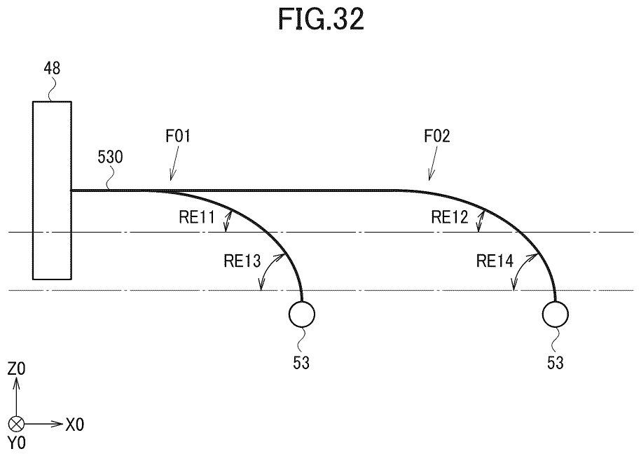

The description continues on the assumption that the direction that the endoscope distal end faces becomes parallel to the direction along the inner wall, in other words, the X0 axis. For example, in the medical manipulator 500 having a configuration in which the distal end thereof bends and the root thereof moves forward/backward, an angle formed by the distal end of the treatment tool 50 located at the distal end of the medical manipulator 500 on an axis in parallel with an axis on which the medical manipulator 500 moves forward/backward is identical independently of a degree of forward/backward movement, but becomes different depending on a degree of bending. The distal end of the treatment tool 50 will be hereinafter referred to as a “treatment tool distal end”. Specifically, for example, as conceptually illustrated in FIG. 32, assume that the third medical manipulator 530 is moved forward/backward in a direction that is parallel to the X0 direction and is bent in the −Z0 direction at a position indicated by F01 or a position indicated by F02. In this case, an angle of RE11 and an angle RE12 in FIG. 32 are equal because degrees of forward/backward movement are different from each other but degrees of bending are equal to each other. Similarly, an angle RE13 and an angle RE14 in FIG. 32 are also equal to each other. In contrast, the angle of RE11 and the angle RE13 in FIG. 32 are different from each other because degrees of forward/backward movement are equal to each other but degrees of bending are different from each other. Similarly, the angle RE12 and the angle RE14 in FIG. 32 are also different from each other.

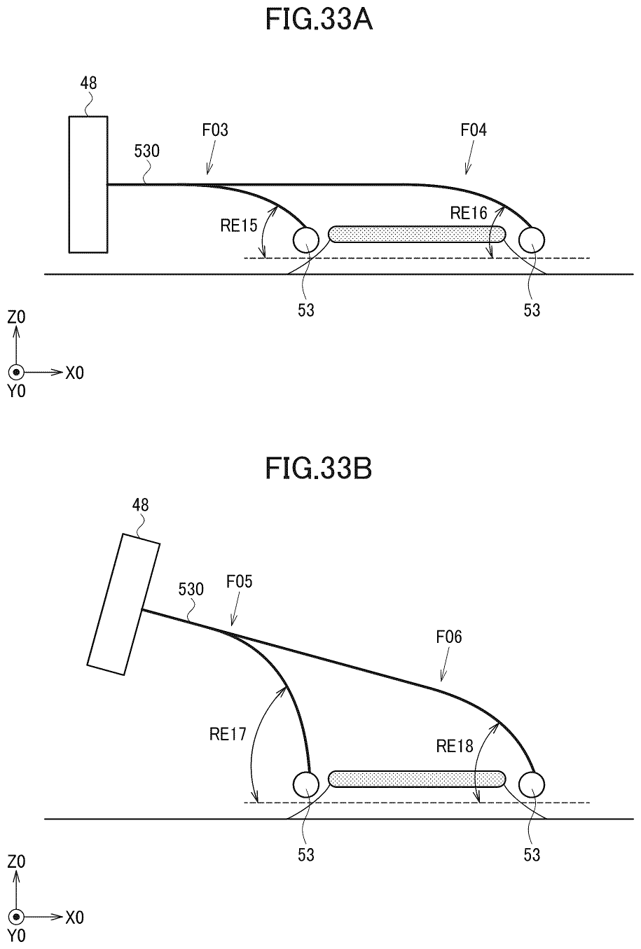

Then, for example, as described above with reference to FIG. 31B, assume that the orientation of the endoscope distal end becomes parallel to the direction along the inner wall, whereby the third medical manipulator 530 is moved forward in the direction parallel to the X0 direction. Additionally, as illustrated in FIG. 33A, assume that the third medical manipulator 530 is bent at a position indicated by F03 or a position indicated by F04. In this case, as described above with reference to FIG. 32, an angle RE15 is equal to an angle RE16 in FIG. 33A. Hence, if the angle RE15 is within an appropriate range for performing the first manipulation (step S10), it is appropriate to cause the third treatment tool 53 to reach the whole of the treatment target illustrated in FIG. 33A.

In contrast, for example, as illustrated in FIG. 33B, assume that the third medical manipulator 530 is moved forward in a state where the orientation of the endoscope distal end is not parallel to the direction along the inner wall and the orientation of the forward movement of the third medical manipulator 530 is not parallel to the X0 direction. Additionally, assume that the third medical manipulator 530 is bent at a position indicated by F05 or a position indicated by F06. In this case, an angle RE17 is larger than an angle RE18 in FIG. 33B. Hence, for example, even in a case where the angle RE18 is within the appropriate range for performing the first manipulation (step S10), there is a possibility that the angle RE17 is not within the appropriate range for performing the first manipulation (step S10).

In this manner, in a case where the endoscope 40 in the present embodiment is used, making the orientation of the endoscope distal end parallel to the direction along the inner wall makes it possible to extend a range of the treatment target being reachable by the treatment tool 50 within an appropriate angle range. This makes it possible to reduce the number of movements of the position of the endoscope 40 to perform the first manipulation (step S10). Note that, while the description has been given using the third medical manipulator 530 and the third treatment tool 53 as an example in FIGS. 32 and 33, the same applies to the other medical manipulators 500 and the other treatment tools 50.

Additionally, the orientation of the endoscope distal end is not necessarily parallel to the direction along the inner wall. In a situation illustrated in FIG. 33B described above, if both the angle RE17 and the angle RE18 are found to be within the appropriate range for performing the first manipulation (step S10), the orientation of the endoscope distal end may not be parallel to the direction along the inner wall.

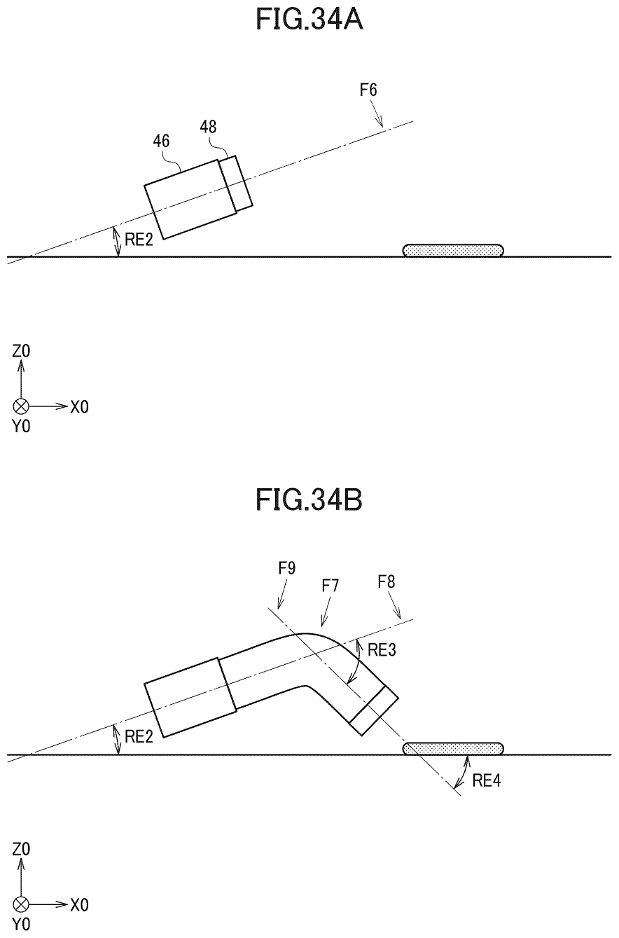

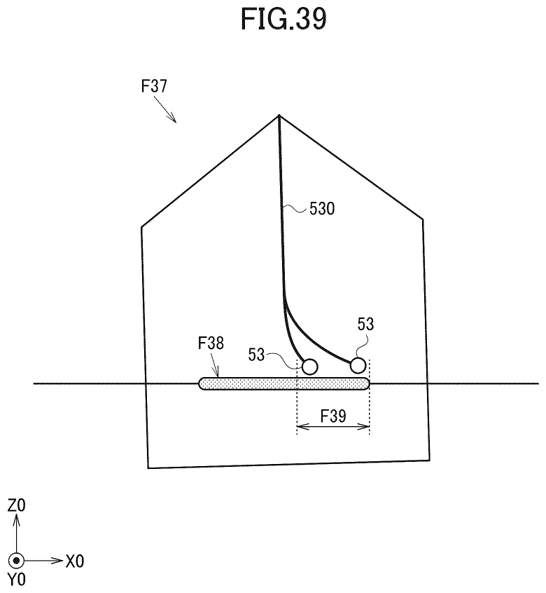

Additionally, there is also a conceivable case where the orientation of the endoscope distal end cannot be made parallel to the direction along the inner wall. For example, as illustrated in FIG. 34A, assume a situation where the direction that the distal end of the overtube 46 faces is an upward direction with respect to the direction along the inner wall. In this case, a straight line indicated by F6 forms an angle RE2 with respect to the direction along the inner wall. The straight line indicated by F6 is a straight line in a direction that is identical to the direction that the distal end of the overtube 46 faces. In this situation, for example, as indicated by F7 in FIG. 34B, bending the endoscope bending portion downward by an angle RE3 makes it possible to form an angle RE4 between a direction that is indicated by F9 and that the endoscope distal end faces and the direction along the inner wall. Note that the angle RE3 is an angle formed between a direction that is indicated by F8 and that the distal end of the overtube 46 faces and the direction that is indicated by F9 and that the endoscope distal end faces. Additionally, there is a relationship of RE4=RE3−RE2 among the angle RE2, the angle RE3, and the angle RE4 described above. In this manner, the situation illustrated in FIG. 34B is not an optimum state to perform the first manipulation (step S10). However, in a case where it is possible to direct the distal end of the treatment tool 50 so as to be appropriate for performing the first manipulation (step S10), there is a possibility that the method of the present embodiment is applicable. Details will be described later with reference to FIG. 39 and the like.

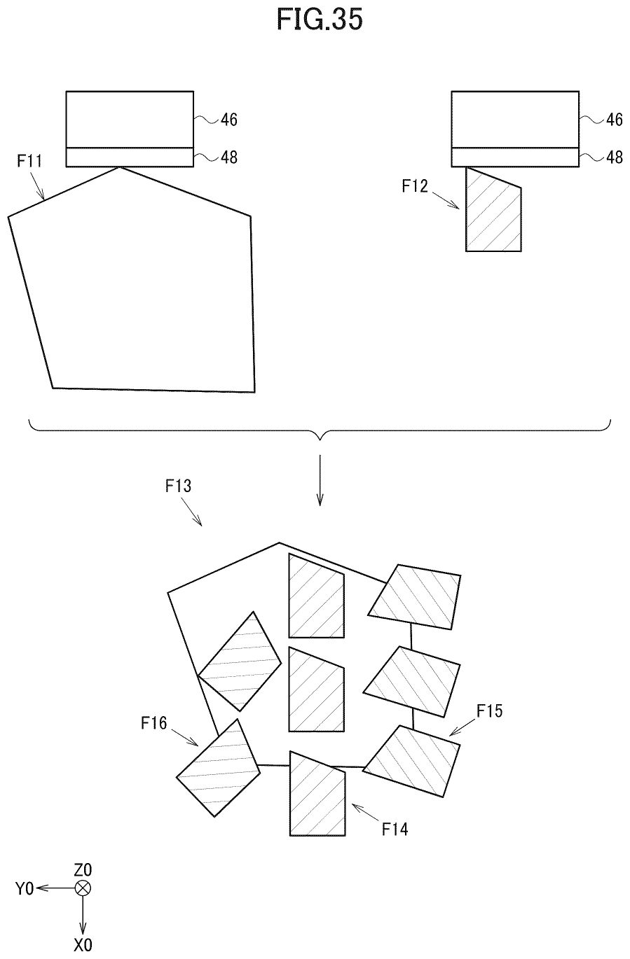

Subsequently, step S240 described above with reference to FIG. 16 is conceptually described with reference to FIG. 35. A region indicated by F11 in FIG. 35 corresponds to the movable range of the endoscope distal end indicated by E64 in FIG. 26. Additionally, a region indicated by F12 in FIG. 35 corresponds to the first work region indicated by E92 in FIG. 29. The processor 100 then adds first work regions to a region indicated by F13 at predetermined intervals in step S240. The region indicated by F13 corresponds to the movable range of the endoscope distal end similarly to the region indicated by F11. With this processing, a first first work region group is set. Note that FIG. 35 illustrates that a plurality of work regions is not superimposed on one another for explanatory convenience, but the first work regions are superimposed on one another at shorter predetermined intervals. It is sufficient if the user determines the length of predetermined intervals as appropriate in consideration of the number of first work regions to be added to the movable range.

Note that the orientation of each first work region is different depending on a position in the movable range of the endoscope distal end. For example, a direction that the first work region indicated by F14 faces, a direction that the first work region indicated by F15 faces, and a direction that the first work region indicated by F16 faces are different from one another.

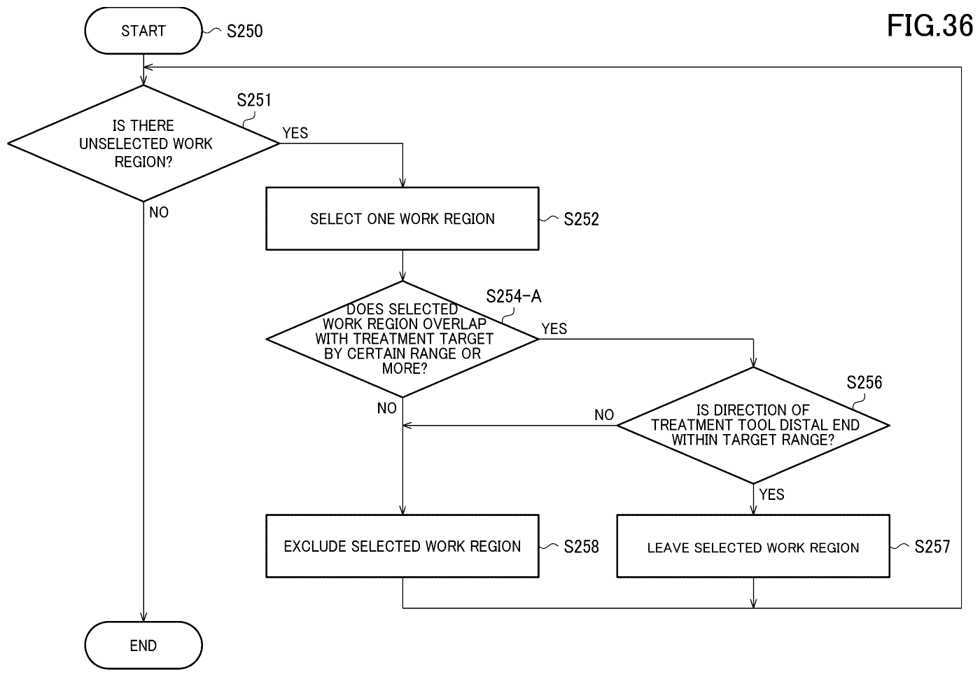

FIG. 36 is a flowchart describing step S250 in detail. The processor 100 performs processing of determining whether there is an unselected work region (step S251). In a case where there is the unselected work region (YES in step S251), the processor 100 selects one work region (step S252). The processor 100 then determines whether the selected first work region overlaps with the treatment target by a certain range or more (step S254-A). Then, in a case of determining that the selected first work region does not overlap with the treatment target by the certain range or more (NO in step S254-A), the processor 100 excludes the selected first work region (step S258) and performs step S251 again.

In contrast, in a case of determining the selected first work region overlaps with the treatment target by the certain range or more (YES in step S254-A), the processor 100 determines that the direction of the treatment tool 50 is within a target range (step S256). Then, in a case of determining that the direction of the treatment tool 50 is within the target range (YES in step S256), the processor 100 leaves the selected work region (step S257) and performs step S251 again. In contrast, in a case of determining that the direction the treatment tool distal end faces is not within the target range (NO in step S256), the processor 100 excludes the selected work region (step S258) and performs step S251 again. Then, in a case of selecting all the work regions, the processor 100 determines that it is NO in step S251 and ends the flow.

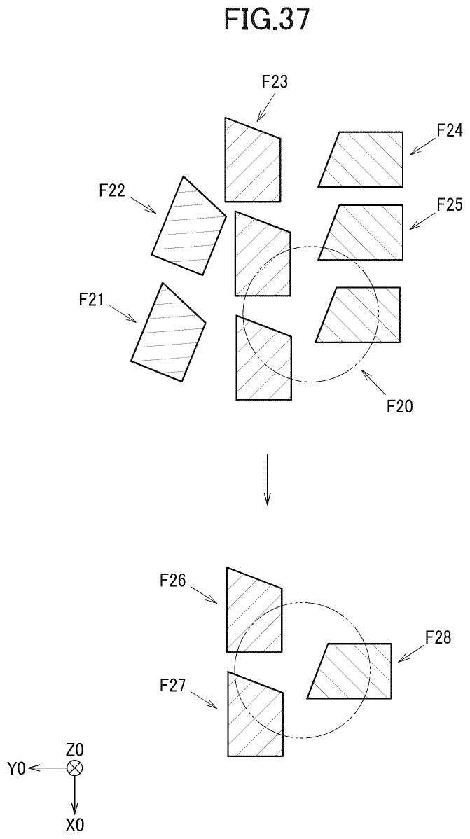

Step S254-A is now described in detail. For example, in a case where a region indicated by F20 in FIG. 37 is determined as the treatment target region, first work regions indicated by F21 to F25 are considered as not overlapping with the treatment target region by the certain range or more. More specifically, for example, the processor 100 makes comparison between the position information regarding the treatment target and stored in step S140, and the position information regarding the first work regions and stored in step S231-B. In a case where an overlap of a set of overlapping position information is within the certain range, the processor 100 determines that it is NO in step

S254. Accordingly, the first work regions indicated by F21 to F25 are eliminated in step S258. Additionally, for example, the processor 100 determines that it is YES in step S254-A with respect to the first work regions F26 to F28 in FIG. 37. Note that FIG. 37 illustrates an example in the X0-Y0 plan view, and, when consideration is further given to the Z0 direction, there is a case where the processor 100 determines that it is NO in step S256, which will be described below, even if determining that it is YES in step S254-A, and the first work region is excluded in subsequent step S258.

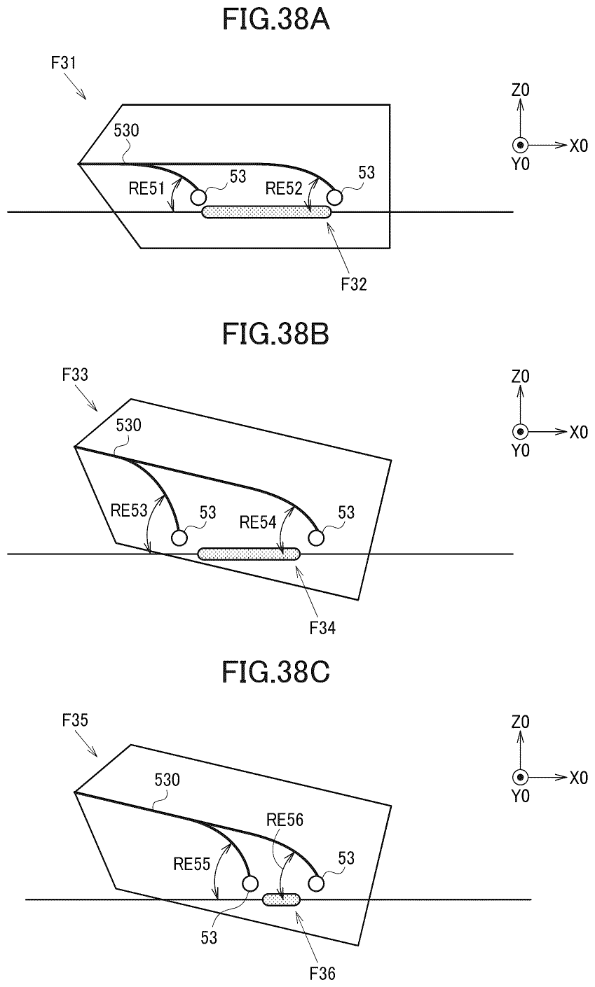

Step S256 is now described in detail. Whether the treatment tool distal end is within the target range is determined in consideration of various factors. Specifically, for example, as illustrated in FIG. 38A, in a case where a direction that the first work region indicated by F31 faces can be regarded as being substantially parallel to the X0 axis, an angle RE51 and an angle RE52 are equal to each other similarly to the case described above with reference to FIG. 33A. In a case where the angle RE51 (=the angle RE52) is within the range appropriate for the first manipulation (step S10), it is appropriate to perform the first manipulation (step S10) on the whole of the treatment target indicated by F32. Thus, the processor 100 determines that it is YES in step S256 with respect to the first work region indicated by F31.

Additionally, for example, as illustrated in FIG. 38B, in a case where a direction that a first work region indicated by F33 faces is different from the direction of the X0 axis, an angle RE53 and an angle RE54 are different from each other similarly to the case described above with reference to FIG. 33B. Hence, for example, the processor 100 determines that the angle RE54 is within the range that is appropriate for performing the first manipulation (step S10), but the angle RE53 is not within the range that is appropriate for performing the first manipulation (step S10). In this case, since it is not appropriate to perform the first manipulation (step S10) on the whole of the treatment target indicated by F34, the processor 100 determines that it is NO in step S256 with respect to the first work region indicated by F33.

Note that, whether it is YES or NO in step S256 is not necessarily determined unambiguously from the orientation of the first work region and the direction of the X0 axis. For example, as illustrated in FIG. 38C, since a direction that a first work region indicated by F35 faces is similar to the direction that the first work region indicated by F33 faces, an angle RE55 and an angle RE56 are different from each other. However, for example, there can be a case where both the angle indicated by RE55 and the angle indicated by RE56 are within the range appropriate for performing the first manipulation (step S10) due to a reason such as a treatment target indicated by F36 having a small size. In this case, the processor 100 determines that it is YES in step S256 with respect to the first work region indicated by F33.

Note that each of the examples illustrated in FIGS. 38A to 38C is an example of determining whether the orientation of the distal end of the third treatment tool 53 is appropriate for the whole of components of the treatment target in the X0 direction, but processing in step S256 is not limited thereto. For example, if the direction that the treatment target distal end faces is appropriate with respect to a continuous portion of the whole of the treatment target, the processor 100 may determine that it is YES in step S256.

For example, as illustrated in FIG. 39, in a case where a direction that a first work region indicated by F37 faces is largely different from the direction of the X0 axis, it is not realistic to control the direction that the distal end of the third treatment tool 53 faces to be within the appropriate range with respect to the whole of components of a treatment target indicated by F38 in the X0 direction. Hence, for example, in a case where the direction that the third treatment tool 53 faces is within the appropriate range for performing the first manipulation (step S10) in a continuous range indicated by F39, the processor 100 may determine that it is YES in step S256 with respect to the first work region indicated by F37. In this case, for example, in a case of performing the first manipulation using the first work region indicated by F37, the user pays attention to incising only a line associated with the region indicated by F39.

Note that each of the examples in FIGS. 38 and 39 is an example of the case where step S256 is applied to the first manipulation (step S10), but a similar way of thinking can also be applied to the case where step S256 is applied to the second manipulation (step S20).

In this manner, step S250 described in detail in FIG. 36 is performed, whereby the second work region group is set based on the first work region group that is set by being mechanically added to the movable range of the endoscope distal end. In the case where the method of the present embodiment is applied to the first manipulation (step S10), the second work region group set in step S250 is hereinafter specifically referred to as a “first second work region group”. Note that, as described later, in the case where the method of the present embodiment is applied to the second manipulation (step S20), the second work region group extracted in step S250 is hereinafter referred to as a “second second work region group”.

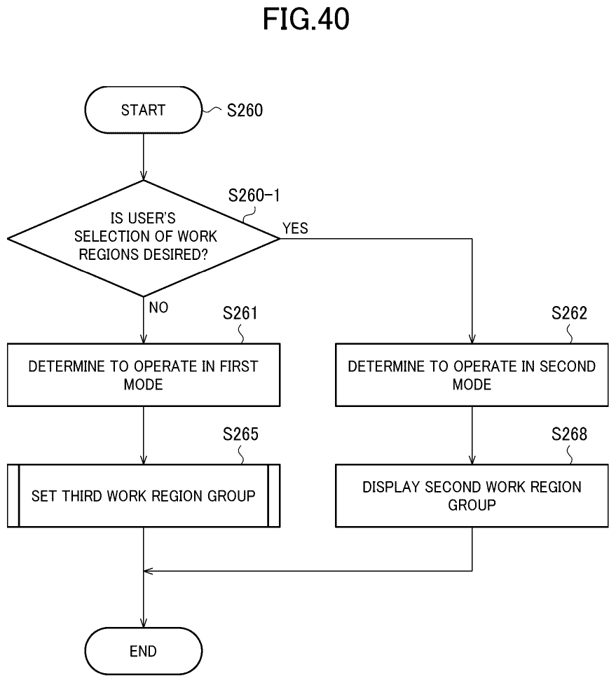

The processing example in step S260 is now described in detail with reference to FIG. 40. The user determines whether the user wants the user's selection of work regions (step S260-1). In a case of not wanting the user's selection of work regions (NO in step S260-1), the user determines to operate the medical manipulator system 5 in a first mode (step S261). Thereafter, the processor 100 sets a third work region group (step S265). Details of S265 will be described later. Note that, in a case where step S261 is performed, the processor 100 makes determination in steps S310, S320, and S330, which have been described above with reference to FIG. 14. That is, the first mode can also be called an automatic mode, and processing in subsequent steps after step S261, which will be described later, is automatically performed.

In contrast, in a case of wanting the user's selection of work regions (YES in step S260-1), the user determines to operate the medical manipulator system 5 in a second mode (step S262). For example, the processor 100 displays the second work region group set in step S250 on a touch panel 620. The user then uses a touch panel function to perform an operation of selecting the first work regions and the like. Then, after completion of selection of the first work regions, the user performs an operation of the operating section or the like to sequentially perform processing in step S310 or subsequent steps. The operating section is not illustrated, that is, the second mode can also be called a manual mode,

Note that, in the present embodiment, selection of the first mode or the second mode in step S260 is performed after the second work region group is set in step S250, but the order is not limited thereto. For example, steps S250 and S265 may be performed after selection of the first mode. In this case, in a case of selecting the second mode, the user performs an operation of selecting individual work regions that constitute the third work region group from the first work region group.

A third work region group in the case where the method of the present embodiment is applied to the first manipulation (step S10) is hereinafter referred to as a “first third work region group”. Similarly, a third work region group in the case where the method of the present embodiment is applied to the second manipulation (step S10) is referred to as a “second third work region group”.

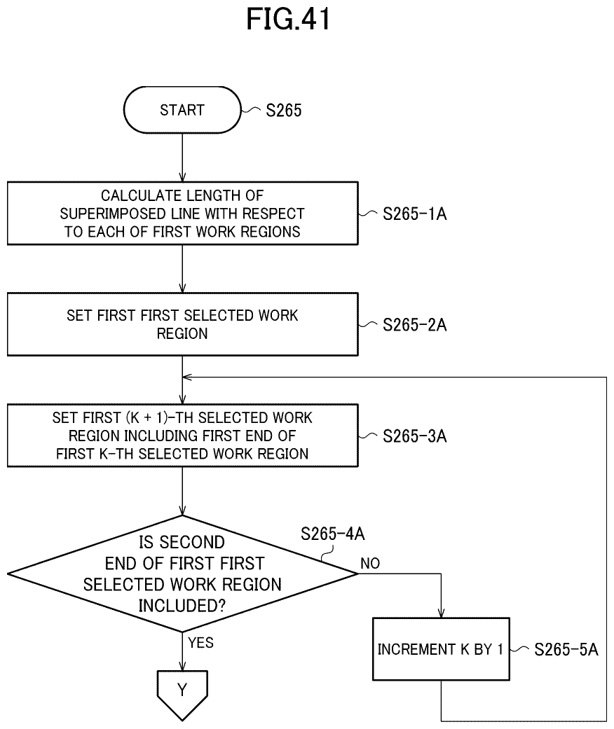

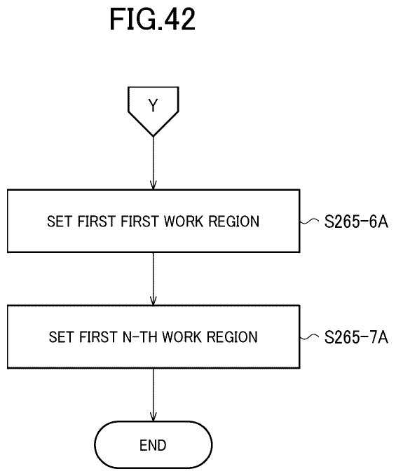

Step S265 is described in more detail with reference to FIGS. 41 to 45. FIGS. 41 and 42 are flowcharts each describing a detailed processing example of processing in step S265. Step S265 is processing of setting, out of the first second work region group set in step S250, the first third work region group obtained by extraction of only work regions necessary for the manipulation, and setting the order of first work regions among the first third work region group. A work region that is selected at the time of execution of step S265 is hereinafter referred to as a “selected work region”. More specifically, a selected work region in the case where the method of the present embodiment is applied to the first manipulation (step S10) is referred to as a “first selected work region”. Similarly, a selected work region in a case where the method of the present embodiment is applied to the second manipulation (step S20) is referred to as a “second selected work region”.

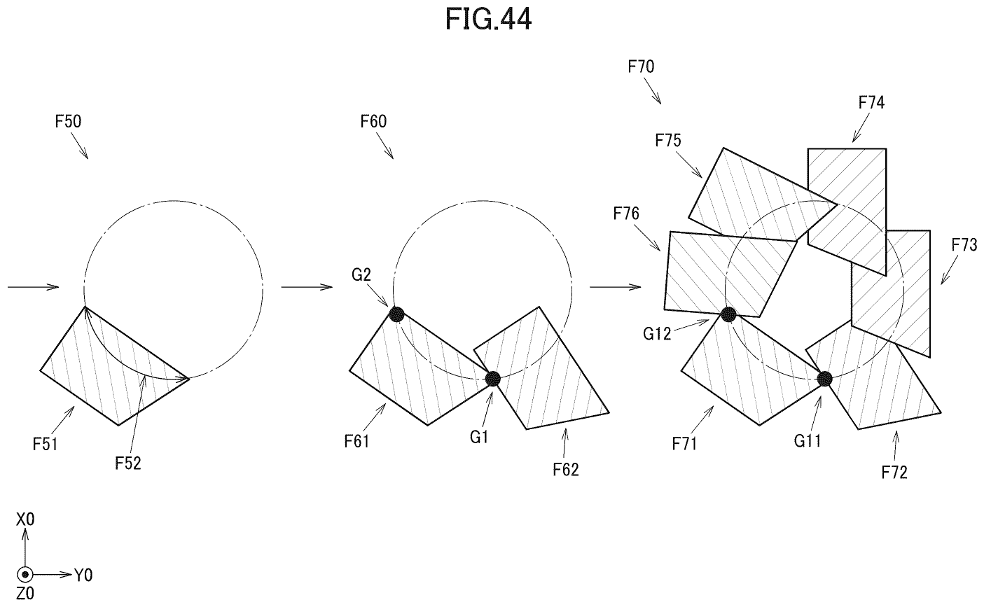

The processor 100 calculates a length of a superimposed line with respect to each of the first selected work regions (step S265-1A). The length of the superimposed line mentioned herein is a line on which the boundary line of the treatment target and the first selected work region are superimposed on each other. A specific example will be described later with reference to FIG. 44. Additionally, the processor 100 stores data that associates each first selected work region and the calculated length of the superimposed line with each other in the memory. Note that the length of the superimposed line may be defined as a boundary line between the second selected work region, which will be described later, and the treatment target, and details will be described later.

The processor 100 then sets a first first selected work region (step S265-2A). For example, the processor 100 selects the longest superimposed line from among superimposed lines calculated in step S265-1A, and selects a first selected work region associated with the selected superimposed line as the first first selected work region. Note that the first first selected work region is not necessarily matched with the first first work region.

Thereafter, the processor 100 sets a first (K+1)-th selected work region including a first end of a first K-th selected work region (step S265-3A). For example, in a case of K=1, the processor 100 sets a first second selected work region including the first end of the first first selected work region set in step S265-2A. More specifically, for example, the processor 100 sets the first selected work region including the first end of the first selected work region and having the longest superimposed line as the second selected work region. The first end mentioned herein is an end on one side, out of two ends of the superimposed line. Note that an end on the other side, out of the two ends of the superimposed line, is referred to as a second end. Additionally, processing that is performed by the processor 100 to store data indicating positional coordinates associated with the first end and data indicating positional coordinates associated with the second end may be included in the above-mentioned step S265-2A.

The processor 100 then determines whether the superimposed line associated with the first K-th selected work region includes the second end of the first first selected work region (step S265-4A). Then, in a case of determining that the superimposed line associated with the K-th selected work region does not include the second end of the first selected work region (NO in step S265-4A), the processor 100 increments K by 1 and performs step S265-3 again. In a case of determining that the superimposed line associated with the K-th selected work region includes the second end of the first selected work region (YES in step S265-4A), the processor 100 performs processing in step S265-6A or subsequent steps, which will be described later. A range of K associated with step S265-3A and step S265-5A is 1≤K≤N. That is, N first selected work regions are brought into a selected state in steps S265-3A, S265-4A, and S265-5A.

Then, in a case of determining that the superimposed line associated with the K-th selected work region includes the second end of the first selected work region (YES in step S265-4A), the processor 100 sets the first first work region (step S265-6A). For example, the processor 100 performs processing of calculating a distance between positional coordinates of the endoscope distal end associated with the first selected work region and positional coordinates of the actually located endoscope distal end. The processor 100 then sets the first selected work region associated with the shortest distance out of calculated distances as the first first work region.

The processor 100 then sets a first N-th work region (step S265-7A). More specifically, the processor 100 sets a first second work region, . . . , and the first N-th work region. With this processing, the order of the first work regions is set.

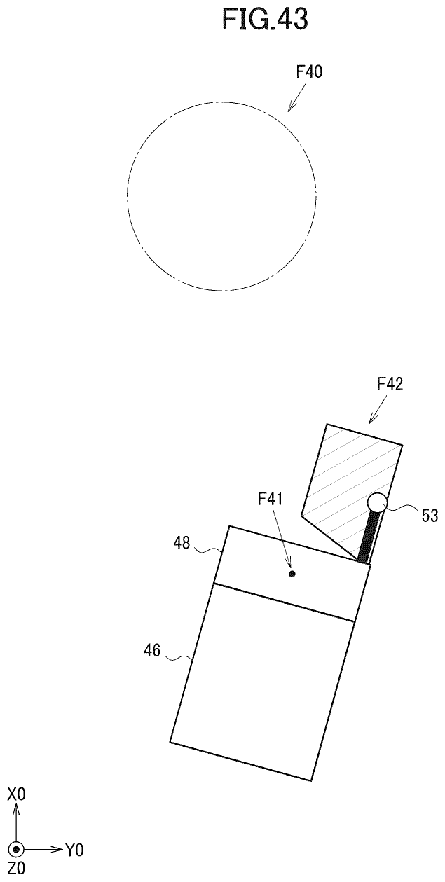

For example, assume that the treatment target is located at a position indicated by F40 and the endoscope distal end is located at a position indicated by F41 in FIG. 43. Additionally, assume that a region indicated by F42 is the first work region calculated in step S230. Although not illustrated, assume that the first second work region group is set as a result of execution of the above-mentioned steps S240 and S250, the user selects the first mode (NO in step S260-1, step S261), and step S265 is performed. Then, the first first selected work region is set in step S265-1A as conceptually indicated by F50 in FIG. 44. More specifically, a superimposed line indicated by F52 is determined as the longest superimposed line, and a first work region indicated by F51 is set as the first first selected work region. Note that the processor 100 may perform display that is similar to that indicated by F50 on the display 610 or the touch panel 620. The same applies to display indicated by F60 and display indicated by F70.

Thereafter, the first second selected work region is set in step S265-3A, for example, as indicated by F60 in FIG. 44. Specifically, for example, a first end indicated by G1 and a second end indicated by G2 are set in a first first selected work region indicated by F61. Then, a first work region indicated by F62 includes the first end indicated by G1 and is the work region with the longest superimposed line, and thus is set as the first second selected work region. Note that, since the second selected work region indicated by F62 does not include the second end indicated by G2, the processor 100 determines that it is NO in step S265-4A.

In this manner, in the medical manipulator system 5 in the present embodiment, the processor 100 sets the selected work region with the longest length that is superimposed on the boundary line of the treatment target as the first selected work region. Additionally, the processor 100 sets the selected work region that includes the first end as one end of the boundary line included in the first selected work region, and that has the longest length that is superimposed on the boundary line as the second selected work region. This makes it possible to minimize the number of selected work regions that are needed for setting the third work region group. As a result, it is possible to minimize the number of movements of the endoscope 40 in the manipulation. This allows the user to efficiently perform the manipulation.

Then, by repeated execution of steps S265-3A, S265-4A, and S265-5A, for example, processing as indicated by F70 in FIG. 44 is performed. Similarly to the above-description, a first first selected work region indicated by F71 includes a first end indicated by G11 and a second end indicated by G12, and a first second selected work region indicated by F72 includes the first end indicated by G11. Then, a first third selected work region indicated by F73, a first fourth selected work region indicated by F74, a first fifth selected work region indicated by F75, and a first sixth selected work region indicated by F76 are further set. Additionally, since a first sixth selected work region indicated by F76 includes the second end indicated by G12, the processor 100 determines that it is YES in step S265-4A.

In this manner, in an example illustrated in FIG. 44, in steps S265-1A to S265-5A, the first first selected work region to the first sixth selected work region are further selected from the first second work region group set in step S250, whereby the first third work region group is set. That is, K associated with steps S265-3A and S265-5A is 6.

For all these reasons, in the medical manipulator system 5 in the present embodiment, when selecting the N-th selected work region (first sixth selected work region) including the second end, which is the other end of the boundary line included in the first selected work region, the processor 100 sets a plurality of selected work regions including the first to N-th selected work regions as the third work region group. This makes it possible to satisfy a condition to end selection of selected work regions that are needed for constituting the third work region group.

Note that in the first manipulation (step S10), as indicated by F70 in FIG. 44, a region on which the first first selected work region to first sixth selected work region are superimposed does not necessarily include the whole treatment target region. That is because, in the first manipulation (step S10), it is sufficient if the user is able to perform incision with the third treatment tool 53 along the boundary line of the treatment target. In other words, the first first selected work region to the first sixth selected work region need to be disposed along the boundary line of the treatment target. That is, in the medical manipulator system 5 in the present embodiment, the processor 100 selects the work regions to be used for the manipulation from among the work regions included in the second work region group as the selected work regions and disposes the plurality of selected work regions along the boundary line of the treatment target to set the third work region group. This makes it possible to appropriately set the third work region group based on the second work region group.

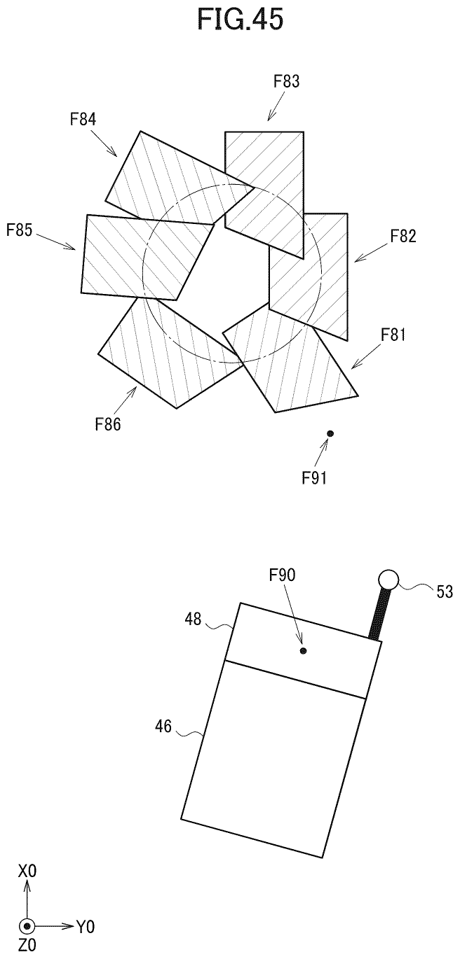

The processor 100 then performs, for example, processing as illustrated in FIG. 45 in steps S265-6A. Specifically, the processor 100 sets a first selected work region that is the closest to the position of the endoscope distal end and that is indicated by F81 from the first first selected work region to the first sixth selected work region as the first first work region. More specifically, for example, positional coordinates of the endoscope distal end corresponding to the selected work region indicated by F81 are associated with a position indicated by F91 in step S240. Although illustration is omitted, the same applies to selected work regions indicated by F82 to F86. Since the position indicated by F91 is the closest to the position of the endoscope distal end indicated by F90, the processor 100 then sets the first selected work region indicated by F81 as the first first work region, and the position indicated by F91 as the first first position.

Thereafter, the first second selected work region to the first N-th work region are set in step S265-7A. A rule of setting the first second work region is only required to be determined as appropriate by the user. For example, in a case where the first second work region to the first N-th work region are set in the order in a counter-clockwise direction when viewed from the first first work region in the +Z0 direction, the processor 100 sets the first selected work region indicated by F82 in FIG. 45 as the first second work region, and sets a first second position (not illustrated). Similarly, the processor 100 sets the first selected work region indicated by F83 as the first third work region, and sets a first third position (not illustrated). Similarly, the processor 100 sets the first selected work region indicated by F84 as the first fourth work region, and sets a first fourth position (not illustrated). Similarly, the processor 100 sets the first selected work region indicated by F85 as a first fifth work region, and sets a first fifth position (not illustrated). Similarly, the processor 100 sets the first selected work region indicated by F86 as a first sixth work region, and sets a first sixth position (not illustrated).

In this manner, in the medical manipulator system 5 in the present embodiment, the processor 100 calculates a length of a portion where the boundary line of the treatment target and the work region are superimposed on one another, and sets a predetermined work region group (first third work region group) including first work regions based on a work region having the largest length among the plurality of work regions. This makes it possible to reduce the number of first work regions that constitute the predetermined work region group (first third work region group). As a result, it is possible to reduce the number of movements of the endoscope distal end in the first manipulation (step S10). This makes it possible to increase work efficiency in the first manipulation (step S10).

Note that the method of the present embodiment may be implemented as a control method. That is, the control method of the present embodiment includes calculating the length of the portion where the boundary line of the treatment target and the work region are superimposed on one another, and setting the predetermined work region group (first third work region group) including first work regions based on the work region having the largest length among the plurality of work regions.

Additionally, in a case where the first second work region is set in the order in a clockwise direction when viewed from the first first work region in the +Z0 direction, the processor 100 sets the first selected work region indicated by F86 in FIG. 45 as the first second work region. Similarly, the processor 100 sets the first selected work region indicated by F85 as the first third work region, the first selected work region indicated by F84 as the first fourth work region, the first selected work region indicated by F83 as the first fifth work region, and the first selected work region indicated by F82 as the first sixth work region. Although illustration is omitted, the first first position, the first second position, the first third position, the first fourth position, the first fifth position, and the first sixth position are similarly set.

Additionally, for example, the order of work regions may be set from a selected work region that is the closest to the present position of the endoscope distal end. In this case, for example, the processor 100 sets the first selected work region indicated by F82 as the first second work region. Similarly, the processor 100 sets the first selected work region indicated by F86 as the first third work region, the first selected work region indicated by F85 as the first fourth work region, the first selected work region indicated by F83 as the first fifth work region, and the first selected work region indicated by F84 as the first sixth work region. Although illustration is omitted, the first first position, the first second position, the first third position, the first fourth position, the first fifth position, and the first sixth position are similarly set.

Thereafter, the processor 100 performs steps S310, S320 and S330 in FIG. 14 to control the driving device 20 from the first first position to the first sixth position and automatically move the endoscope 40.

In this manner, the first work regions in the first first work region group set in step S240 are reduced to be set as the first second work region group in step S250. Furthermore, the first work regions in the first second work region group set in step S250 are reduced to be set as the first third work region group in step S265, and the first first work region is set among the first work regions included in the first third work region group. For all these regions, in the medical manipulator system 5 in the present embodiment, the processor 100 calculates information regarding the work region with respect to each of a plurality of positions in the movable range of the endoscope 40 to set the first work region group (step S240), and based on the first work region group, sets a plurality of work regions that is superimposed on the treatment target region as the second work region group (step S250). Additionally, the processor 100 sets, based on the second work region group, a plurality of work regions to be used for the manipulation as the third work region group, and sets, based on the third work region group, the work region that is the closest to the position of the endoscope 40 as the first work region (step S265). This makes it possible to reduce the number of work regions in a stepwise manner. As a result, it is possible to accurately select the work region that is appropriate for efficiently performing the manipulation.

Details of the case where the processing example in FIG. 14 is applied to the second manipulation (step S20) are now described. Note that, in this case, details of step S100 in FIG. 15, step S210 in FIG. 16, and step S220 in FIG. 16 are similar to the case where the method of the present embodiment is applied to the first manipulation (step S20), and thus a description thereof is omitted.

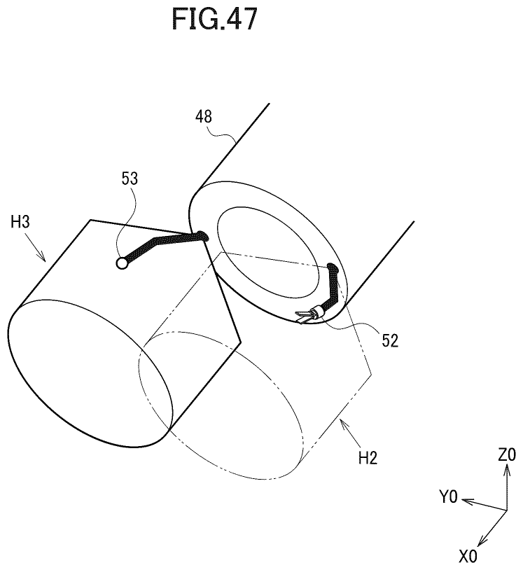

The flowchart in FIG. 46 is a detailed processing example of step S230 in a case where the method of the present embodiment is applied to the second manipulation (step S20). The processor 100 sets a second space region based on an operation range of the second treatment tool 52 (step S232). Thereafter, the processor 100 sets a third space region based on an operation range of the third treatment tool 53 (step S233). Thereafter, the processor 100 calculates a second work region based on the second space region and the third space region (step S234). Note that step S233 in FIG. 46 is processing that is common to step S231-A in FIG. 27 in terms of the operation range of the third treatment tool 53. That is, the third space region in step S233 is similar to the first space region in step S231-A. That is, an outline of the third space region is as indicated by H3 in FIG. 47.

Step S232 is now described on a conceptual basis. As described above, the second medical manipulator 520 that drives the second treatment tool 52 is configured to be capable of moving forward/backward and bending with a mechanism that is similar to a mechanism in the endoscope 40 and the third treatment tool 53. Hence, an outline of the second space region is as indicated by H2 similarly to the outline indicated by H3.

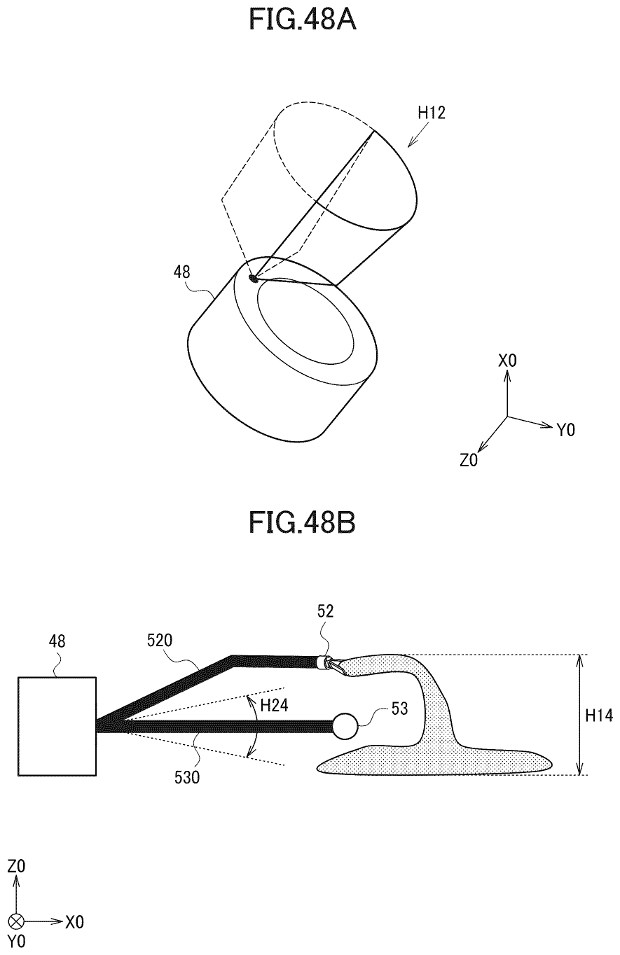

Step S234 is now described on a conceptual basis. In the second manipulation (step S20), the second treatment tool 52 grasps the treatment target, and an incision operation using the third treatment tool 53 is performed. Then, as indicated by H12 in FIG. 48A, it is preferable that the second treatment tool 52 be allowed to be located in a region between a twelve o'clock direction and a six o'clock direction when the second space region is viewed from the direction that the endoscope distal end faces. Note that the twelve o'clock direction mentioned herein is a direction of twelve o'clock in a case where the second space region is regarded as the clock face of a 12-hour clock when viewed from the direction that the endoscope distal end faces. Similarly, the six o'clock direction mentioned herein is a direction of six o'clock in a case where the second space region is regarded as the clock face of the 12-hour clock when viewed from the direction that the endoscope distal end faces. Additionally, the second manipulation (step S20) requires an operation of grasping the treatment target with the second treatment tool 52 and pulling the treatment target upward in the +Z0 direction. Thus, it is preferable that the second treatment tool 52 be capable of operating, for example, in a range indicated by H14 in FIG. 48B in the Z0 direction. The specific range indicated by H14 is determined as appropriate depending on a case.

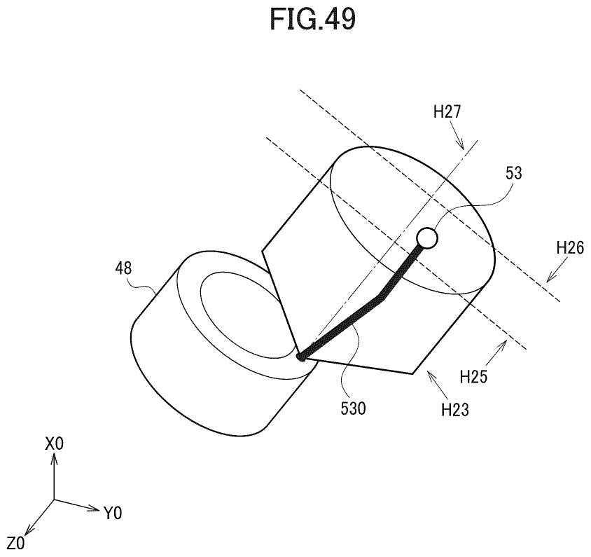

Additionally, in the X0-Z0 plan view, it is preferable that the third treatment tool 53 move in a range indicated by H24 in FIG. 48B using the direction along the X0 direction as a reference in the second manipulation (step S20). The specific range indicated by H24 is determined as appropriate depending on a case. Hence, when a third space region indicated by H23 in FIG. 49 is viewed from the endoscope distal end side, it is preferable that the third treatment tool 53 be located within a range between a dotted line indicated by H25 and a dotted line indicated by H26. Note that the dotted line indicated by H25 and the dotted line indicated by H26 are lines perpendicular to a broken line indicated by H27. The broken line indicated by H27 is a line from twelve o'clock toward six o'clock in a case where the third space region is regarded as the clock face of the 12-hour clock when viewed from the endoscope distal end side. In other words, the dotted line indicated by H25 and the dotted line indicated by H26 are lines in parallel with a direction from three o'clock toward nine o'clock in a case where the third space region is regarded as the clock face of the 12-hour clock when viewed from the endoscope distal end side.

In this manner, since the second manipulation (step S20) is performed in the situation illustrated in FIG. 48B, it is thought to be ideal that the endoscope distal end faces the direction in parallel with the X0 direction as illustrated in FIG. 31B described above, similarly to the first manipulation (step S10). However, also similarly to the first manipulation (step S10), there is a possibility that treatment can be performed by bending or the like of the treatment tool 50 even in a case where the direction that the endoscope distal end faces cannot be made parallel to the X0 direction.

Additionally, although not illustrated in FIGS. 47 to 49, consideration is also given to the second treatment tool and the third treatment tool 53 being visually recognizable in a predetermined range of the endoscope image as described with reference to FIGS. 28A and 28B. Note that it is sufficient if the grasping forceps as the second treatment tool 52, when grasping the treatment target, are located to be visually recognizable in the predetermined range of the endoscope image by the user.

That is, the medical manipulator system 5 in the present embodiment includes a plurality of medical manipulators 500 and respective treatment tools 50 corresponding to the medical manipulators 500. The processor 100 acquires information regarding a work region, which is a range virtually set based on a range that is within a predetermined range in the endoscope image and in which each treatment tool 50 is seen and based on the movable range of each medical manipulator 500. This makes it possible to set the range of the second work region more appropriately.

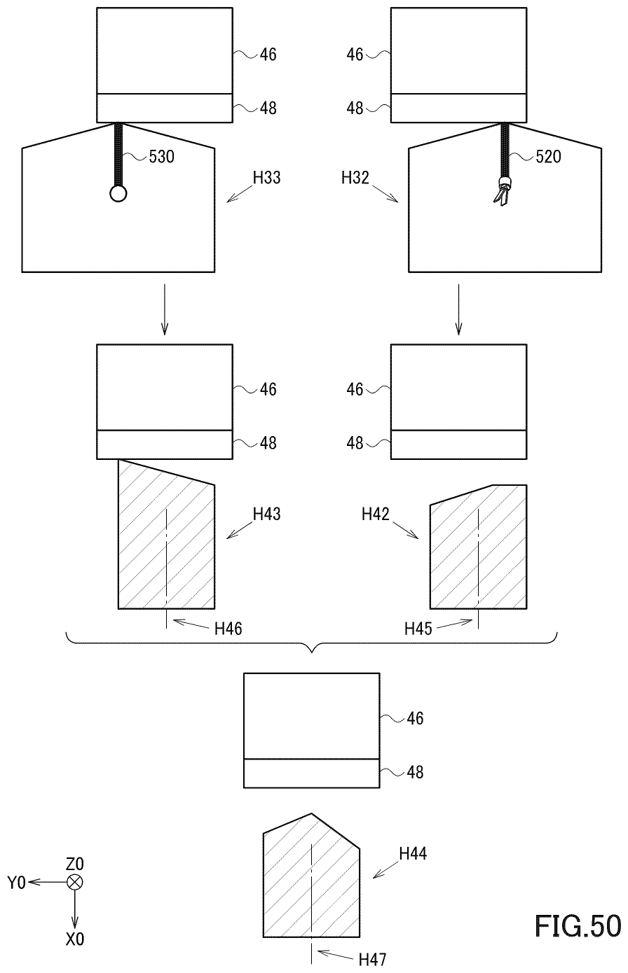

For these reasons described above, a second space region indicated by H32 in FIG. 50 is calculated in step S232, and the second space region is reduced to a region indicated by H42 in step S234. Similarly, a third space region indicated by H33 in FIG. 50 is calculated in step S233, and the third space region is reduced to a region indicated by H43 in step S234. Note that illustration is given as the X0-Y0 plan view in FIG. 50, but the second space region and the third space region are reduced also in the Z0 direction. Although not illustrated, specifically, for example, the second space region and the third space region are reduced in consideration of the relationship between the lens position of the imager 42 and the protrusion position of the medical manipulator 500, the conditions described above with reference to FIGS. 48B and 49, and the like. The relationship between the lens position of the imager 42 and the protrusion position of the medical manipulator 500 has been described above with reference to FIG. 30.

The second work regions finally calculated in step S234 may be selected as appropriate by the user. For example, in a case where all work regions are desired to be automatically determined by the medical manipulator system 5 as described later, it is sufficient if a region indicated by H44 in FIG. 50 serves as a work region. The second work region indicated by H44 is a region on which both the reduced second space region indicated by H42 and the reduced third space region indicated by H43 overlap. Additionally, for example, in a case where the user wants determination about the position appropriate for use of the second treatment tool 52 to be made by the medical manipulator system 5 and wants the user's manual operation of the third treatment tool 53, it is sufficient if the reduced second space region indicated by H42 serves as the second work region. Additionally, for example, in a case where the user wants determination about the position appropriate for use of the third treatment tool 53 to be made by the medical manipulator system 5 and wants the user's manual operation of the second treatment tool 52, it is sufficient if the reduced second space region indicated by H43 serves as the second work region.

Additionally, a direction of a line indicated by H47 with respect to the second work region indicated by H44 may be referred to as a “direction that the second work region faces” for explanatory convenience. Similarly, in a case where the work region indicated by H42 serves as the second work region, a direction of a line indicated by H45 may be referred to as the “direction that the second work region faces”. In a case where the work region indicated by H43 serves as the second work region, a direction of a line indicated by H46 may be referred to as the “direction that the second work region faces”.

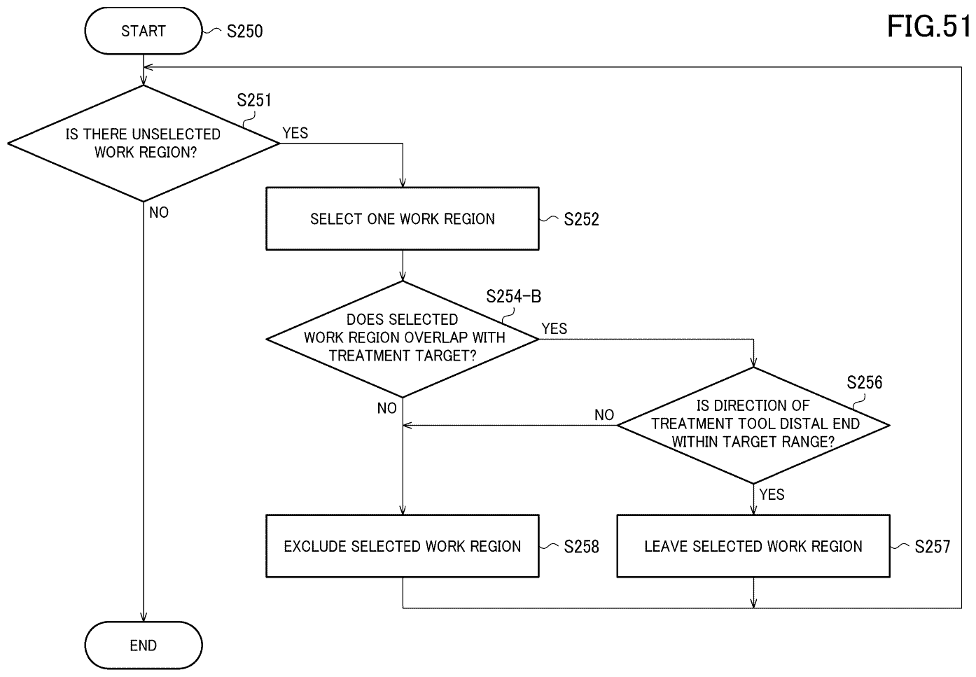

Thereafter, the processor performs step S240 in FIG. 16, and thereafter performs step S250. A description of step S240 is omitted due to a reason that is similar to the reason in step S100 and the like. The flowchart in FIG. 51 is a detailed processing example of step S250 in a case where the method of the present embodiment is applied to the second manipulation (step S20). Note that in the description with reference to FIG. 51, a description of processing (steps S251, S252, S258, S259, and the like) that is common to the flowchart in FIG. 36 is omitted as appropriate.

In a case of determining that it is YES in step S251, the processor 100 performs the above-mentioned step S252, and determines whether the selected second work region overlaps with the treatment target (step S254-B). That is, step S254-B in FIG. 51 is implemented regardless of how much amount the selected second work region overlaps with the treatment target.

Then, in a case of determining that the selected second work region does not overlap with the treatment target (NO in step S254-B), the processor 100 excludes the selected work region (step S258) and performs step S251 again.

In contrast, in a case of determining the selected work region overlaps with the treatment target (YES in step S254-B), the processor 100 determines whether the direction of the treatment tool distal end is within the target range (step S256).

Step S256 in FIG. 51 and step S256 in FIG. 36 are similar in intent. That is, although illustration is omitted, the determination is made based on whether the third treatment tool 53 can reach the treatment target in an angle range indicated by H24 in FIG. 48B. Whether the distal end of the third treatment tool 53 is within the appropriate angle range depends on the relationship between the X0 direction and the direction that the second work region faces, the size of the treatment target, and the like, similarly to the case described above with reference to FIG. 38. Additionally, similarly to the case described above with reference to FIG. 39, in a case where a certain range of an area that is appropriate for performing the second manipulation (step S20) exits, the processor 100 may perform processing of determining that it is YES in step S256 even if the distal end of the third treatment tool 53 is unable to face the whole treatment target within the appropriate angle range.

Then, in a case of determining that the direction of the treatment tool distal end is within the target range (YES in step S256), the processor 100 performs step S257, and thereafter performs step S251 again. In contrast, in a case where the direction that the treatment tool distal end faces is not within the target range (NO in step S256), the processor 100 performs step S258, and thereafter performs step S251 again. In a case of determining that it is NO in step S251, the processor 100 ends the flow. With this processing, the second second work region group including the second work regions that has undergone step S257 is set.

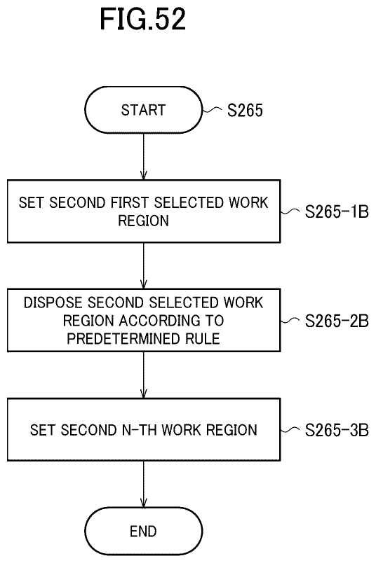

Thereafter, assume that the user does not want the user's selection of work regions, and selects to operate the medical manipulator system 5 in the first mode (NO in step S260-1, step S261). Step S265 in this case is described with reference to FIGS. 52 to 54.

In a flowchart in FIG. 52, the processor 100 sets a second first selected work region from the second second work region group set in step S250 (step S265-1B). For example, the processor 100 selects a second work region that includes the boundary line of the treatment target and that overlaps with the treatment target in a wide range, and sets the second work region as the second first selected work region. The processor 100 then disposes the second selected work region according to a predetermined rule (step S265-2B).

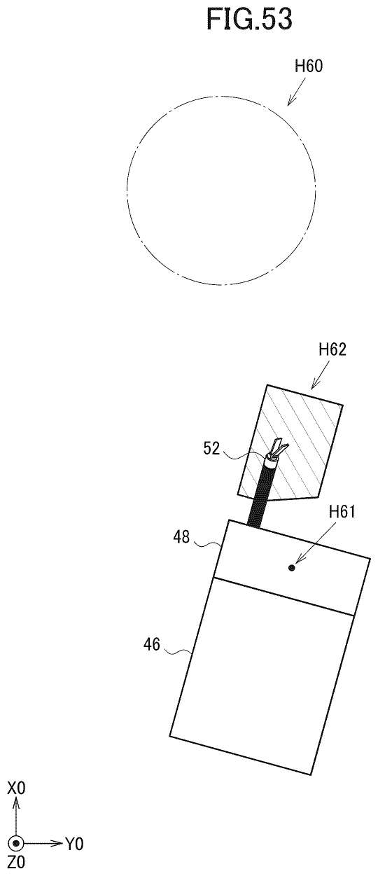

For example, when conceptually illustrated, assume that the treatment target is located at a position indicated by H60 and the endoscope distal end is located at a position indicated by H61 in FIG. 53. Note that a relationship between position information regarding the treatment target indicated by H60 and position information indicated by H61 in FIG. 53 is assumed to be identical to the relationship between the position information regarding the treatment target indicated by F40 and the position information indicated by F41 in FIG. 43. In other words, a situation illustrated in FIG. 53 is a situation where the user performs the first manipulation (step S10) in the situation illustrated in FIG. 43, and thereafter continuously performs the second manipulation (step S20). Note that, in a case where the first manipulation (step S10) is further performed, there is a possibility that the treatment target indicated by H60 in FIG. 53 is somewhat changed in shape or the like in comparison with the treatment target indicated by F40 in FIG. 43, but the treatment target is assumed to have identical three-dimensional shape information before or after the execution of the first manipulation (step S10) in the present embodiment.

Assume that a region indicated by H62 in FIG. 53 is the second work region calculated in step S230, and corresponds to the work region indicated by H42 in FIG. 50. Note that the region indicated by H62 in FIG. 53 may correspond to the second work region indicated by H43 in FIG. 50, or may correspond to the second work region indicated by H44 in FIG. 50.

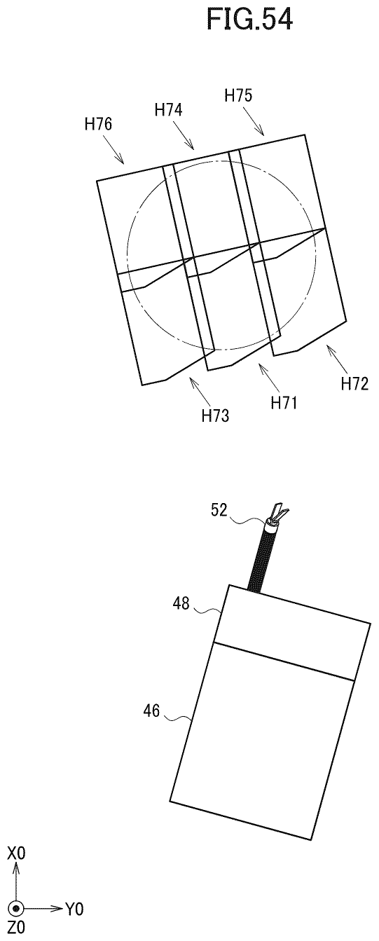

In the situation illustrated in FIG. 53, assume that processing until step S250 is performed and the second second work region group is set. For example, assume that a second work region indicated by H71 in FIG. 54 is selected as the second first selected work region in step S265-1B in FIG. 52. The second work region indicated by H71 includes the boundary line of the treatment target and overlaps the treatment target in a wide range.

Thereafter, a second selected work region indicated by H72, a second selected work region indicated by H73, a second selected work region indicated by H74, a second selected work region indicated by H75, and a second selected work region indicated by H76 are selected and disposed in step S265-2B in FIG. 52. For example, the second selected work regions indicated by H72 to H76 are selected as follows. The respective positions of the second selected work regions indicated by H72 to H76 are set and arrayed at predetermined intervals using the positional coordinates of the endoscope distal end corresponding to the second first selected work region indicated by H71 serving as a reference. Then, the second work region in a case where the set position serves as the position of the endoscope distal end is selected as the second selected work region, and is disposed together with the second first selected work region. The predetermined intervals are set based on a size of the second work region or the like so that the arrayed second work regions are superimposed on one another.

In the ESD as the second manipulation (step S20), the movable range of the second treatment tool 52 and the movable range of the third treatment tool 53 need to reach the whole region of the treatment target. Then, the above-mentioned method in steps S265-1B and S265-2B is automatically performed, whereby it is possible to appropriately select the second selected work regions. Note that the second selected work regions are disposed in a reticular pattern in FIG. 54, but may be disposed in a staggered pattern. The pattern can be determined as appropriate by the user.

Back to the flow in FIG. 52, the description continues. Thereafter, the processor 100 sets a second N-th work region (step S265-3B). More specifically, the processor 100 sets the order of the second first work region to the second N-th work region. The processor 100, for example, performs processing of selecting the position of the endoscope distal end that is the closest to the first first position (the position indicated by F91 in FIG. 45) corresponding to the above-mentioned first first work region (the work region indicated by F81 in FIG. 45) as the second first position, and performs processing of setting the second selected work region associated with the selected second first position as the second first work region. Specifically, for example, in the case of FIG. 54, the processor 100 sets the second selected work region indicated by H72 as the second first work region, and sets the second first position (not illustrated). Note that, in a case where the first manipulation (step S10) is omitted, the second work region associated with the position of the endoscope distal end that is the closest to the present position of the endoscope distal end may be set as the second first work region.

For these reasons above, in the medical manipulator system 5 in the present embodiment, the second first position is, among the positions of the endoscope corresponding to the plurality of second work regions, a position that is the closest to the first first position. This makes it possible to bring the position of start of the second manipulation (step S20) close to the position of start of the first manipulation (step S10). As a result, it is possible to increase work efficiency in the treatment.

The processor 100 then sets a second second work region, a second third work region, a second fourth work region, a second fifth work region, and a second sixth work region. In this manner, the processor 100 sets a set of second work regions including the second first work region to the second sixth work region as a second third work region group.

Since the ESD as the second manipulation (step S20) is manipulation of exfoliating the lesion with the third treatment tool 53 from the front side when viewed from the endoscope distal end, it is thought to be convenient to set the order of the second work regions from the second work region that is the closest to the position of the endoscope distal end. Thus, in the situation illustrated in FIG. 54, the processor 100, for example, sets the second work region indicated by H71 as the second second work region. Similarly, the processor 100 sets the second work region indicated by H73 as the second third work region, the second work region indicated by H75 as the second fourth work region, the second work region indicated by H74 as the second fifth work region, and the second work region indicated by H76 as the second sixth work region. Although illustration is omitted, a second first position, a second second position, a second third position, a second fourth position, a second fifth position, and a second sixth position are also set. Thereafter, the processor 100 ends the flow in step S260, and sequentially moves the endoscope 40 to the second N-position corresponding to each second N-th work region in subsequent steps S310, S320, and S330.

Note that, in substitution for steps S265-1B and S265-2B in FIG. 52, the second first selected work region to the second N-th selected work region may be set by the following method. Although illustration in the flowchart is omitted, for example, the processor 100 calculates a length of a superimposed line and a size of a superimposed area with respect to each second selected work region. The size of the superimposed area is a size of an area in which the treatment target and the second work region are superimposed on each other in the X0-Y0 plan view. Additionally, the processor 100 stores data that associates the length of the calculated superimposed line and the size of the superimposed area with each second work region in the memory.

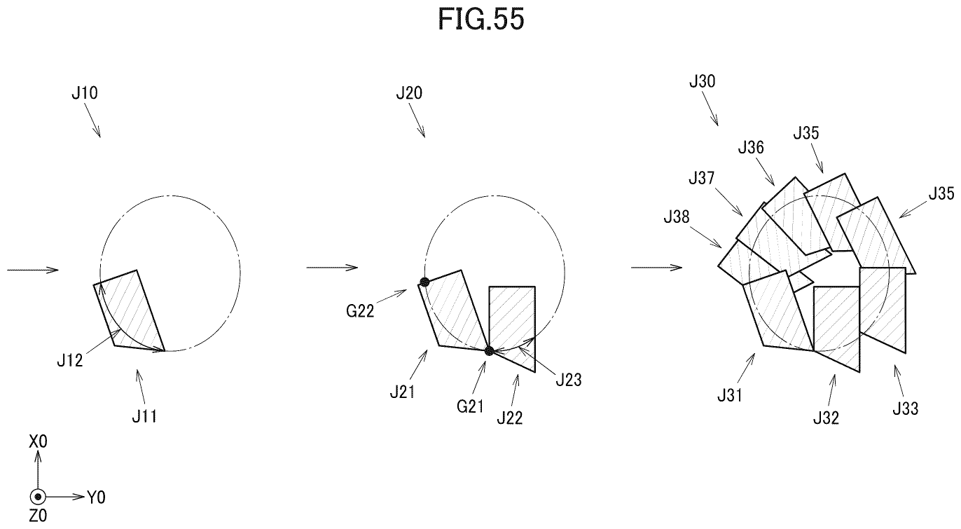

The processor 100 then sets the second first selected work region based on data indicating the length of the superimposed line and the size of the superimposed area and stored in the memory. For example, the processor 100 extracts a predetermined number of pieces of data indicating sizes ranking high from data indicating sizes of superimposed areas. Then, the processor 100 may set, out of the extracted data, the second work region associated with data indicating the longest superimposed line as the second first selected work region. With this processing, for example, a second first selected work region indicated by J11 is set as conceptually illustrated by J10 in FIG. 55. That is, since a superimposed line indicated by J12 is longer than superimposed lines associated with second work regions as the other candidates, the second work region indicated by J11 is selected as the second first selected work region.

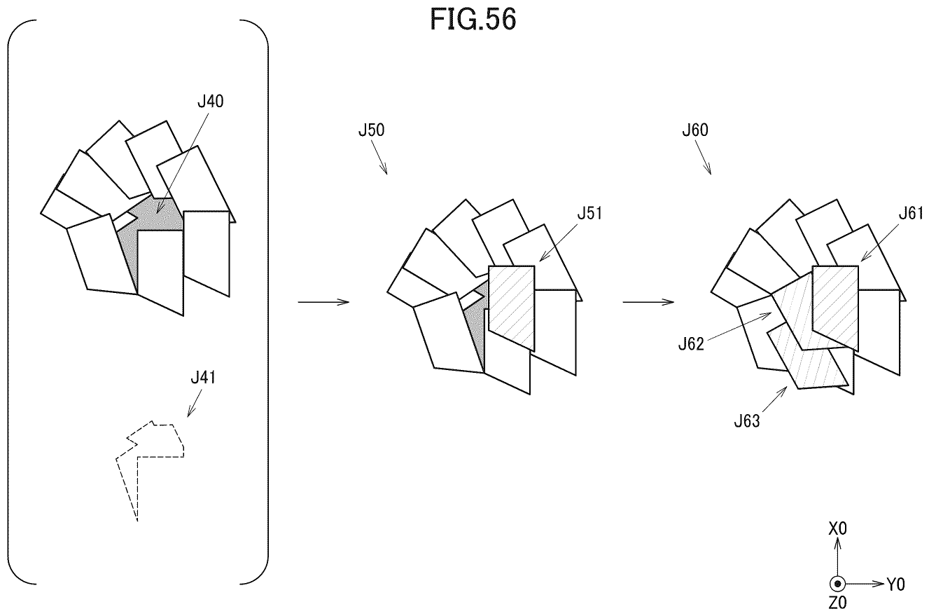

Note that the processor 100 may perform display that is similar to illustration in J10 on the display 610 or the touch panel 620. The same applies to illustration in J20 in FIG. 55, illustration in J30, illustration in J50 in FIG. 56, and illustration in J60. The illustration in J50 will be described later.

Thereafter, the processor 100 sets the second second selected work region as indicated by J20. For example, the processor 100 selects the second first selected work region to set a first end indicated by G21 and a second end indicated by G22. The processor 100 then extracts, from data indicating the size of the superimposed area, a predetermined number of pieces of data indicating sizes ranking high with respect to the second work regions other than the second first selected work region. The processor 100 then sets, out of the extracted data, a second work region that includes the first end indicated by G21 and that is associated with data indicating the longest superimposed line as the second second selected work region. The second selected work regions subsequent to the second third selected work region are similarly set. With this processing, in addition to the second first selected work region indicated by J21, the second first selected work region indicated by J22 is set. That is, since the superimposed line indicated by J23 is longer than the superimposed lines associated with the second work regions as the other candidates, the second work region indicated by J22 is selected as the second second selected work region.

Additionally, the processor 100 determines whether the selected second second selected work region includes the second end indicated by G22. In the example indicated by J20 in FIG. 55, since the second second selected work region indicated by J22 does not include the second end indicated by G22, the processor 100 further performs processing of setting the second third selected work region.

By repeated execution of the above-mentioned method, a situation indicated by J30 in FIG. 55 occurs. That is, a second first selected work region indicated by J31, a second second selected work region indicated by J32, a second third selected work region indicated by J33, a second fourth selected work region indicated by J34, a second fifth selected work region indicated by J35, and a second sixth selected work region indicated by J36, a second seventh selected work region indicated by J37, and a second eighth selected work region indicated by J38 are set. The second eighth selected work region indicated by J38 includes the second end.

The processor 100 then calculates a difference region when the situation indicated by J30 in FIG. 55 occurs. The difference region mentioned herein is, out of the treatment target region, a region on which none of the second work regions selected so far is superimposed in the X0-Y0 plan view, and, for example, a region indicated by J40 in FIG. 56. That is, the processor 100 calculates an area of the difference region indicated by J40 in FIG. 56, and stores data indicating the calculated area in the memory. Note that a dotted line graphic pattern indicated by J41 indicates a contour of the difference region indicated by J40 in an excerpted manner.

Additionally, the processor 100 calculates a length of the contour of the difference region. That is, the processor 100 obtains a set of points that constitute the contour of the difference region indicated by J41 in FIG. 56, and stores data indicating the calculated set of points in the memory.

The processor 100 then further sets the second selected work region based on data indicating the area of the difference region and data indicating the length of the contour of the difference region. For example, the processor 100 calculates a size of the area that is superimposed on the difference region with respect to an unselected second work region. The processor 100 then extracts a predetermined number of pieces of data indicating sizes of areas that are superimposed on the difference region and that rank high. The processor 100 then calculates a size of the contour of the difference region included in the area that is superimposed on the difference region with respect to the second work region associated with each of the extracted data. Then, the processor 100 further sets, out of the obtained lengths of contours of difference regions, a second work region associated with the longest boundary line of the difference region as the second second selected work region.

Specifically, assume that, for example, with the above-mentioned method, a second work region indicated by J51 is selected as indicated by J50 in FIG. 56. With this processing, the second work region indicated by J51 is set as a second ninth selected work region.

Then, by repeated execution of a similar method, as indicated by J60 in FIG. 56, in addition to the second ninth selected work region indicated by J61, a second tenth selected work region indicated by J62, a first tenth selected work region indicated by J62, and a second eleventh selected work region indicated by J63 are further set in the X0-Y0 view. With this processing, the whole treatment target region is superimposed on the set of set second selected work regions.

With the above-mentioned method, the second first selected work region to the second eleventh selected work region for performing the second manipulation (step S20) are selected from the second second work region group set in step S250, and the second third work region group is set. In each of the examples in FIGS. 55 and 56, it is necessary to perform control of the position of the endoscope 40 at least eleven times to perform the second manipulation (step S20).

Note that a method of setting the second third work region group is not limited thereto. Various mathematical methods for efficiently superimposing the second work regions on the area of the treatment target have been proposed, and the mathematical methods may be appropriately adopted in step S265 in FIG. 40.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, components in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

1. A medical manipulator system comprising:
an outer manipulator including an imager that captures an image;
an inner manipulator that protrudes from a distal end surface of the outer manipulator;
a driving device that controls the outer manipulator and the inner manipulator; and
a processor,
wherein the processor
acquires the image in which a treatment target is seen from the imager,
recognizes a region of the treatment target from the image,
acquires three-dimensional shape information regarding a lumen,
acquires three-dimensional shape information regarding the outer manipulator,
calculates information regarding a work region that is an operable range of the inner manipulator with respect to each of a plurality of positions in a movable range of the outer manipulator to acquire information regarding a plurality of the work regions, and
performs drive control of the outer manipulator to move to a first position corresponding to a first work region selected from among the plurality of work regions based on the region of the treatment target, the three-dimensional shape information regarding the lumen, and the three-dimensional shape information regarding the outer manipulator.

2. The medical manipulator system as defined in claim 1, wherein the processor acquires information regarding the work region that is a range virtually set based on a range that is within a predetermined range of the outer manipulator and in which a treatment tool corresponding to the inner manipulator is seen and based on a movable range of the medical manipulator.

3. The medical manipulator system as defined in claim 1, further comprising a plurality of the inner manipulators and respective treatment tools corresponding to the plurality of inner manipulators,
wherein the processor acquires information regarding the work region that is a range virtually set based on a range that is within a predetermined range of the image and in which each of the treatment tools is seen and based on movable ranges of the plurality of respective inner manipulators.

4. The medical manipulator system as defined in claim 1, wherein the processor superimposes a movable range of the inner manipulator and a range that is within a certain range appropriate for disposition of a treatment tool associated with the inner manipulator in the image and in which the treatment tool associated with the inner manipulator is seen on each other, and thereby acquires information regarding the work region that is a range virtually set.

5. The medical manipulator system as defined in claim 1,
wherein the processor
calculates a length of a portion in which a boundary line of the treatment target and the work region are superimposed on each other, and
sets, among the plurality of work regions, a predetermined work region group including the first work region based on the work region with the largest length of the portion.

6. The medical manipulator system as defined in claim 1,
wherein the processor
calculates information regarding the work region with respect to each of a plurality of positions in a movable range of the outer manipulator to set a first work region group,
sets, based on the first work region group, a plurality of the work regions that is superimposed on the region of the treatment target as a second work region group,
sets, based on the second work region group, a plurality of the work regions to be used for manipulation as a third work region group, and
sets, based on the third work region group, a work region that is closest to a position of the outer manipulator as the first work region.

7. The medical manipulator system as defined in claim 6,
wherein the processor
selects, from among work regions included in the second work region group, a work region to be used for the manipulation as a selected work region, and
disposes a plurality of the selected work regions along a boundary line of the treatment target to set the third work region group.

8. The medical manipulator system as defined in claim 7,
wherein the processor
sets the selected work region that has a largest length of a portion that is superimposed on the boundary line of the treatment target as a first selected work region, and
sets the selected work region that includes a first end as one end of the boundary line included in the first selected work region and that has the largest length of the portion that is superimposed on the boundary line, as a second selected work region.

9. The medical manipulator system as defined in claim 8, wherein the processor sets, when selecting an N-th selected work region including a second end as another end of the boundary line included in the first selected work region, a plurality of the selected work regions including the first selected work region to the N-th selected work region as a third work region group.

10. The medical manipulator system as defined in claim 1,
wherein the processor
selects the first work region and a second work region from among a plurality of the work regions, and
moves the outer manipulator to the first position corresponding to the selected first work region, and thereafter moves the outer manipulator to a second position corresponding to the selected second work region.

11. The medical manipulator system as defined in claim 1,
wherein the processor
performs drive control of a position of the outer manipulator to move to a first first position corresponding to a first first work region selected from among a plurality of first work regions in first manipulation, and
performs drive control of the position of the outer manipulator to move to a second first position corresponding to a second first work region selected from among a plurality of second work regions in second manipulation.

12. The medical manipulator system as defined in claim 11, wherein, among positions of the outer manipulator corresponding to the plurality of second work regions, the second first position is a position that is closest to the first first position.

13. The medical manipulator system as defined in claim 1, further comprising:
a first sensor; and
a second sensor,
wherein the processor
acquires the three-dimensional shape information regarding the lumen from the first sensor, and
acquires the three-dimensional shape information regarding the outer manipulator from the second sensor.

14. The medical manipulator system as defined in claim 13, wherein
the first sensor is a distance sensor that employs a time of flight (TOF) method or a light detection and ranging (LiDAR) method, and
the second sensor is a bending shape observation sensor that employs a sensing method using a magnetic field or a sensing method using distortion.

15. A processor that controls an outer manipulator including an imager that captures an image, an inner manipulator that protrudes from a distal end surface of the outer manipulator, and a driving device that controls the outer manipulator and the inner manipulator,
wherein the processor
acquires the image in which a treatment target is seen from the imager,
recognizes a region of the treatment target from the image,
acquires three-dimensional shape information regarding a lumen,
acquires three-dimensional shape information regarding the outer manipulator,
calculates information regarding a work region that is an operable range of the inner manipulator with respect to each of a plurality of positions in a movable range of the outer manipulator to acquire information regarding a plurality of the work regions, and
performs drive control of the outer manipulator to move to a first position corresponding to a first work region selected from among the plurality of work regions based on the region of the treatment target, the three-dimensional shape information regarding the lumen, and the three-dimensional shape information regarding the outer manipulator.

16. A control method of controlling an outer manipulator including an imager that captures an image, an inner manipulator that protrudes from a distal end surface of the outer manipulator, and a driving device that controls the outer manipulator and the inner manipulator, the method comprising:
acquiring the image in which a treatment target is seen from the imager;
recognizing a region of the treatment target from the image;
acquiring three-dimensional shape information regarding a lumen;
acquiring three-dimensional shape information regarding the outer manipulator;
calculating information regarding a work region that is an operable range of the inner manipulator with respect to each of a plurality of positions in a movable range of the outer manipulator to acquire information regarding a plurality of the work regions; and
performing drive control of the outer manipulator to move to a first position corresponding to a first work region selected from among the plurality of work regions based on the region of the treatment target, the three-dimensional shape information regarding the lumen, and the three-dimensional shape information regarding the outer manipulator.

17. The control method as defined in claim 16, further comprising acquiring information regarding the work region that is a range virtually set based on a range that is within a predetermined range of the image and in which a treatment tool corresponding to the inner manipulator is seen and based on a movable range of the inner manipulator.

18. The control method as defined in claim 16, further comprising:
calculating a length of a portion in which a boundary line of the treatment target and the work region are superimposed on each other; and
setting, among the plurality of work regions, a predetermined work region group including the first work region based on the work region with the largest length of the portion.

19. The control method as defined in claim 16, further comprising:
selecting the first work region and a second work region from among a plurality of the work regions; and
moving the outer manipulator to the first position corresponding to the selected first work region, and thereafter moving the outer manipulator to a second position corresponding to the selected second work region.

20. The control method as defined in claim 16, further comprising:
performing drive control of a position of the outer manipulator to move to a first first position corresponding to a first first work region selected from among a plurality of first work regions in first manipulation; and
performing drive control of the position of the outer manipulator to move to a second first position corresponding to a second first work region selected from among a plurality of second work regions in second manipulation.
